(19) United States
(12) Patent Application Publication
(10) Pub. No.: US 2005/0266519 A1
(43) Pub. Date: Dec. 1, 2005

(54) Methods and compositions for modulating and detecting activin dimer and dimer formation

(75) Inventors: Sally Mellor, Sandringham (AU); Gail Risbridger, East Malvern (AU); Nigel Groome, Oxford (GB); Mark Cranfield, Oxford (GB); Emma Ball, South Yarra (AU); Anne O'Connor, Clayton (AU); David Phillips, Clayton (AU); Jacqui Schmitt, Clayton (AU); Jean Francois Ethier, Clayton (AU)

Correspondence Address: DARBY & DARBY P.C., P. O. BOX 5257, NEW YORK, NY, 10150-5257, US

(73) Assignee: Prostate Diagnostics Pty. Ltd. (PDPL)

(21) Appl. No.: 10/318,283

(22) Filed: Dec. 12, 2002

Related U.S. Application Data
(60) Provisional application No. 60/340,782, filed on Dec. 12, 2001.
(60) Provisional application No. 60/340,783, filed on Dec. 12, 2001.

(51) Int. Cl. A61K 38/17; C12P 21/02; C12N 5/06; C12N 15/85

(52) U.S. Cl. 435/69.1; 435/320.1; 435/325; 514/12; 435/455

(57) ABSTRACT

Methods and compositions for modulating activin dimer formation, such as the formation of activin dimers formed by the dimerisation of activin subunits β_{A}, β_{B}, β_{C}, β_{D}, or β_{E}, or combinations thereof, are provided. The invention also relates to methods and compositions for detecting an activin monomer or dimer using, for example, an antibody. Methods and compositions for diagnosing and/or prognosing, preventing or treating conditions and/or diseases associated with activin dimer formation, such as prostate cancer, are disclosed.

### FIELD OF THE INVENTION

The present invention relates to methods and compositions for modulating activin dimer formation, such as the formation of activin dimers formed by the dimerisation of activin subunits β_{A}, β_{B}, β_{C}, β_{D} or β_{E}, or combinations thereof. The invention also relates to methods or compositions for detecting an activin monomer or dimer. The invention also provides methods and compositions for diagnosing and/or prognosing, preventing or treating conditions and/or diseases associated with activin dimer formation, such as a prostate cancer.

### BACKGROUND

Activins, are members of the TGF-β superfamily that have diverse roles as potent growth and differentiation factors in many organs and tissues. Activins are homo- or heterodimers of activin β subunits, such as β_{A}, β_{B}, β_{C}, β_{D} or β_{E} that form activin dimer ligands. The activin family encompasses disulfide-linked dimeric proteins characterized by a conserved cysteine-knot motif. Activin was originally isolated in ovarian follicular fluid as a stimulator of FSH secretion, however it is now recognised that activins have a range of biological activities that include mesoderm induction in Xenopus laevis embryos, immune suppression, bone growth, nerve cell survival, wound healing, tumourogenesis and tissue differentiation in pancreas, kidney and heart (1-4).

Most activin family members appear to be involved in differentiation and control of proliferation. Examples of activin dimer ligands include activin A (β_{A}-β_{A}), activin B (β_{B}-β_{B}), and heterodimer activin AB (β_{A}-β_{B}). More recently, activin β_{C} subunit, along with activin β_{D} and β_{E} subunits, have been identified, which form a different subset of activin β subunits, but no biological function of activin C (β_{C}-β_{C}) has been identified.

The activin β_{C} subunit was cloned from mouse (5) and human liver (6), however expression has also been identified in ovary and testis (7). Activin β_{D} has been cloned from Xenopus and microinjection of β_{D} cDNA induced mesoderm induction, however no mammalian equivalent has been identified (8). Activin β_{E} subunit was cloned from mouse liver (9) and found to be expressed in rat liver and lung (10). Zhang and others demonstrated differences in β_{A} and β_{C} mRNA regulation following rat partial hepatectomy and proposed that activin β_{C} was a liver chalone (11, 12). However, no biological role for activins C, D or E has been established. Activin β_{C}-β_{C} forms the activin C homodimer (19), however the formation of β_{C} activin heterodimers has not been confirmed.

Activin signal transduction is initiated by ligand binding inducing the formation of a heteromeric receptor complex of type I and II transmembrane serine/threonine kinase receptors. Activin binding to ActRII or IIB, results in recruitment and phosphorylation of type I receptor ActRI, thereby initiating the phosphorylation of downstream signaling proteins, the Smad (Sma- and Mad-related) proteins. Following phosphorylation, Smad2 and 3 (receptor-regulated Smads), form a heteromeric complex with Smad4 (co-Smad) and translocate from the cytoplasm to the nucleus (13-15). Interaction of Smad proteins with either transcription factors or DNA-binding elements regulate appropriate gene expression. For example, in Xenopus, the DNA binding transcription factor, forkhead activin signal transducer-1 (FAST-1) binds to the Smad2 and Smad4 complex to activate the activin response element (ARE) on the Xenopus Mix.2 promoter (16, 17). It is not known if activin β_{C} and β_{E} subunits transduce a signal through the above activin receptors or if they have their own receptors.

Little is known about the formation of activin dimers and the regulation of activin dimer formation. In particular, the regulation of dimerisation of activin subunits β_{A}, β_{B}, β_{C}, β_{D} or β_{E}, or combinations thereof. Consequently, there remains a need for providing effective methods and compositions for modulating activin dimer formation.

### SUMMARY OF THE INVENTION

In a first aspect of the invention there is provided a method of modulating the formation of an activin dimer in a cell or biological sample, the method including controlling levels or bioactivity of activin β_{C} in the cell or biological sample. Preferably, the method includes modulating the formation of activin dimers formed by the dimerisation of activin subunits selected from the group consisting of β_{A}, β_{B}, β_{C}, β_{D} or β_{E}, or combinations thereof.

The method preferably includes modulating the formation of activin homodimers selected from the group consisting of activin A (β_{A}-β_{A}), activin B (β_{B}-β_{B}), activin C (β_{C}-β_{C}), activin D (β_{D}-β_{D}) or activin E (β_{D}-β_{E}). The method may preferably include modulating the formation of activin heterodimers selected from the group consisting of activin AB (β_{A}-β_{B}), activin AC (β_{A}-β_{C}), activin AD (β_{A}-β_{D}), activin AE (β_{A}-β_{E}), activin BC (β_{B}-β_{C}), activin BD (β_{B}-β_{D}), activin BE (β_{B}-β_{E}), activin CD (β_{C}-β_{D}), activin CE (β_{C}-β_{E}) or activin ED (β_{E}-β_{D}). Most preferably, the method includes modulating the formation of activin A, activin B, activin C, activin D or activin E.

In a preferred aspect of the invention there is provided a method of inhibiting the formation of an activin dimer in a cell or biological sample, the method including increasing levels or bioactivity of activin β_{C} in the cell or biological sample.

The activin dimers that are inhibited from forming are preferably selected from the group consisting of activin A (β_{A}-β_{A}), activin B (β_{B}-β_{B}), activin D (β_{D}-β_{D}), activin E (β_{D}-β_{E}), activin AB (β_{A}-β_{B}), activin AD (β_{A}-β_{D}), activin AE (β_{A}-β_{E}), activin BD (β_{B}-β_{D}), activin BE (β_{B}-β_{E}), or activin ED (β_{E}-β_{D}). Most preferably, the method includes modulating the formation of activin A, activin B, activin C, activin D, or activin E. In the method, activin β_{C} levels or bioactivity are preferably increased by delivering an amount of activin β_{C} in the cell or biological sample or increasing the expression of activin β_{C} in the cell or biological sample.

The invention preferably provides a method of inducing the formation of an activin dimer in a cell or biological sample, the method including decreasing levels or bioactivity of activin β_{C} in the cell or biological sample. The activin dimers that are induced to form are preferably selected from the group consisting of activin A (β_{A}-β_{A}), activin B (β_{B}-β_{B}), activin D (β_{D}-β_{D}), activin E (β_{E}-β_{E}), activin AB (β_{A}-β_{B}), activin AD (β_{A}-β_{D}), activin AE (β_{A}-β_{E}), activin BD (β_{B}-β_{D}), activin BE (β_{B}-β_{E}), or activin ED (β_{E}-β_{D}) Most preferably, the method includes inducing the formation of activin A, activin B or activin C. Preferably, levels or bioactivity of activin β_{C} are decreased by an activin β_{C} inhibitory molecule such as an antibody against activin β_{C}, an activin β_{C} antisense oligonucleotide or an agent that decreases the expression or bioactivity of activin β_{C}.

In another aspect of the invention there is provided a purified antibody, wherein the antibody recognises an epitope of an activin β_{C} subunit. Preferably, the antibody is capable of recognising monomeric or dimeric forms of activin β_{C}. More preferably, the antibody recognises an epitope of activin β_{C} that includes the amino acid sequence VPTARRPLSLLYYDRDSNIVKTDIPDMVVEAC. It is preferred that the antibody is a monoclonal antibody. Preferably, the antibody is specific to an activin β_{C} subunit. More preferably, the antibody is specific to the human activin β_{C} subunit.

The activin β_{C} antibody of the present invention may be used in a number of methods and diagnostic and/or prognostic techniques. For instance, the activin β_{C} antibody of the present invention may be used in ELISA, immunohistochemistry, immunoaffinity purification, immunoprecipitation, Western Blot and antibody binding studies.

In another aspect of the invention there is provided a method of detecting an activin β_{C} subunit and/or an activin dimer including an activin β_{C} subunit, wherein the method includes detecting an activin β_{C} subunit and/or an activin dimer including an activin β_{C} subunit with an antibody that recognises an epitope of an activin β_{C} subunit.

In another aspect of the invention there is provided a method of diagnosing and/or prognosing a disease or condition associated with activin dimer formation, the method including detecting an activin β_{C} subunit and/or an activin dimer including an activin β_{C} subunit in a cell or biological sample of a subject. Preferably, the method includes the use of an antibody that recognises an epitope of an activin β_{C} subunit to detect an activin β_{C} subunit and/or an activin dimer including an activin β_{C} subunit in a cell or biological sample of a subject.

In a further aspect of the invention there is provided a method of diagnosing and/or prognosing a disease or condition associated with activin dimer formation, the method including detecting levels or bioactivity of activin β_{C} and/or an activin β_{C} dimer in a cell or biological sample of a subject. Preferably, the activin β_{C} dimer detected is activin AC (β_{A}-β_{C}), activin BC (β_{B}-β_{C}), activin C (β_{C}-β_{C}), activin CD (β_{C}-β_{D}) or activin CE (β_{C}-β_{E}). Most preferably, the activin β_{C} dimer detected is activin AC (β_{A}-β_{C})

A further aspect of the invention is a method of treating or preventing a disease or condition associated with activin dimer formation, the method including controlling levels or bioactivity of activin β_{C} in a subject such that activin dimer formation in the subject is modulated. Preferably the disease or condition is prostate cancer.

In a preferred aspect of the invention there is provided a method of diagnosing and/or prognosing a disease or condition associated with activin dimer or dimer formation in a subject, the method including detecting an activin β_{C} dimer with an antibody that recognises an epitope of an activin β_{C} subunit in a cell or biological sample of the subject.

In the methods of the present invention, the disease or condition associated with activin dimer formation may preferably include diseases or conditions of the liver, prostate, pancreas, kidney, heart, reproductive organs, skeletal muscle, ovary, testis, brain and neural tissue, adrenal gland, pituitary, thyroid gland, stomach, colon, lung, urinary bladder, endometrium, breast, lymph node, skin, salivary gland, bone, nasal cavity, duodenum, gallbladder, uterine cervix, thymus, placenta, fallopian tube, uterus, tonsil, spleen, appendix, seminal vesicle, larynx, tongue, pituitary, small intestine, rectum, esophagus, myometrium, and soft tissue. Preferably the disease is cancer or a tumour. Most preferably the disease is prostate cancer or liver disease

In another aspect of the present invention there is provided a method for detecting a propensity for an activin dimer to form in a cell or biological sample, said method comprising detecting a level or bioactivity of activin β_{C} in the cell or biological sample.

In another aspect of the present invention there is provided a pharmaceutical composition for treating, preventing or diagnosing and/or prognosing a disease or condition associated with activin dimer formation, the composition including an effective amount of activin β_{C} or an activin β_{C} inhibitory molecule, and a suitable pharmaceutically acceptable diluent, excipient or carrier. Preferably, the pharmaceutical composition includes an activin β_{C} inhibitory molecule and is suitable for treating prostate cancer or liver disease.

### FIGURES

FIG. 1, consisting of FIGS. 1A and 1B, shows the comparison of the effects of activin A, B and C on DNA synthesis by LNCaP and activin C on PC3 human prostate tumour cells. LNCaP (A) and PC3 (B) cells were plated and cultured in DMEM and 5% FCS, and the media was replenished on day 3 with 40 ng/mL activin A, activin B or activin C or matching vehicle buffer controls. (A) Exogenous addition of activin A and activin B inhibited the DNA synthesis of LNCaP cells. Activin C had no effect on the proliferation of these cells. (B) PC3 cells were not responsive to Activin C. Each value represents the mean±SD from five replicate wells. Significance P<0.0001.

FIG. 2, consisting of parts A-E, shows the comparison of the effects of activin A, B and C on activin responsive promoters. CHO cells and LβT2 cells were transiently transfected with activin responsive promoters. Cells were incubated with activin A, activin B, or activin C for 24 hours and firefly luciferase activity was quantified and normalised for β-galactosidase. Activin A stimulated p3TP-lux approximately 4.4 fold and activin B approximately 6 fold (part A), AR3-lux was stimulated by activin A approximately 4 fold and activin B approximately 6 fold (part B), gsc-lux was stimulated by activin A approximately 1.3 fold and activin B approximately 1.5 fold (part C), pGL3-5.5oFSHβ was stimulated by activin A approximately 1.6 fold and activin B approximately 2.2 (part D) and 3XGRAS-PRL-lux was stimulated by activin A approximately 3.3 fold and activin B approximately 3.2 fold (part E). Activin C did not stimulate the activin responsive elements. Each value represents mean±SD from three replicate wells.

FIG. 3 shows the expression of activin β_{C} protein in the supernatant of transfected PC3 cells. Western blot analysis of activin β_{C} or control transfected PC3 cell conditioned media. Proteins were separated by 15% SDS-PAGE gel. A 13 kDa band was detected in the hr-activin (human recombinant activin) C positive control (Lane 1; 10 ng, Lane 2; 20 ng, Lane 3; 40 ng) and in conditioned media from activin β_{C} transfected PC3 cells (Lane 6). No band was detected in hr-activin A negative control (Lane 4), conditioned media from HepG2 cell line (Lane 5) or control transfected PC3 cells (Lane 7).

FIG. 4, consisting of graphs A, B and C, shows activin A production, activin AC production and activation of signal transduction pathway by PC3 cells overexpressing activin β_{C}. PC3 cells were transiently cotransfected with activin β_{C} or control vector, ARE (activin response element) and Renilla luciferase reporter construct. Conditioned media and cells were collected at 24, 48 and 72 hrs. The endogenous production of Activin A and Activin AC was measured by ELISA and ARE activation was measured by luciferase assay. (graph A) Conditioned media from PC3 cells overexpressing activin β_{C} produced significantly lower levels of activin A, than controls, at 24, 48 and 72 hour time points. (graph B) Activation of the activin response element was reduced in PC3 cells expressing activin β_{C} as compared to control wells at 24, 48 and 72 hours. (graph C) Activin AC protein was produced in conditioned media from activin β_{C}-transfected PC3 cells between 24 and 72 hrs. No activin AC was detected in control wells. Each value represents mean±SD from five replicate wells.

FIG. 4A, consisting of graphs A1, B1, C1 shows activin AC production, activin A production and activation of activin signal transduction pathway by PC3 cells overexpressing activin β_{C}. PC3 cells were transiently cotransfected with either the activin β_{C} subunit cDNA expression vector (pRK5-β_{C}) or the control vector (pRK5), pAR3-lux and the Renilla luciferase reporter construct. Conditioned media and cells were collected at 24, 48 and 72 hrs after transfection. The endogenous production of activin A homodimeric protein and activin AC heterodimeric protein was measured by ELISA. (A1) Activin AC protein was produced in conditioned media from activin pctransfected PC3 cells with levels increasing in a time-dependent manner. Low levels of activin AC were detected in conditioned media from control cells. Each value represents mean±SD from four replicate wells. (B1) Conditioned media from PC3 cells overexpressing activin β_{C} produced significantly lower levels of activin A, than controls, at 24, 48 and 72 hour time points. (C1) pAR3-lux activity was normalised for transfection efficiency by dividing by the of renilla luciferase activity measured in a dual luciferase assay. Activation of the activin response element was reduced in PC3 cells expressing activin β_{C} as compared to control wells at 24, 48 and 72 hours. Each value represents mean±SD of four replicate wells and is representative of two separate experiments. Groups with different letters are significantly different P<0.05.

FIG. 5 shows activin AC levels in conditioned media from activin β_{C}-transfected PC3 cells and a semipurified bovine follicular fluid (bFF) preparation. Dose-response curves of (a) semipurified bFF preparation (T) and (b) two conditioned media samples from activin Pctransfected PC3 cells (v and δ) are shown as measured by activin AC ELISA. The bFF preperation and media samples, diluted in unconditioned media, diluted out in a linear manner and the slopes were parallel to each other.

FIG. 5A, consisting of graphs A, B and C, shows the results from the development and validation of the activin AC ELISA. (A) The effect of sample pre-treatments on the performance of the activin AC ELISA. Increasing volume of bFF (closed symbols) or conditioned culture media from activin β_{C} transfected PC3 cells (β_{C}PC3-CM) (open symbols) were assayed with a novel AC ELISA with different sample pre-assay treatment: no treatment (square symbols); denaturation with SDS and boiling (triangles) oxidation with H₂O₂ (inverted triangles); combined treatment with denaturation and oxidation (circles). Points represent the mean of duplicate wells. (B) Dose-response curves for bFF interim standard (closed squares) and conditioned culture media from β_{C} transfected PC3 cells (β_{C}PC3-CM) (open squares) using pre-treatment with denaturation and oxidation. Each point represents the mean and standard deviation of n=3 assays. (C) Dose-response curve of bFF standard in the presence (closed squares) and absence (open squares) of 50 ng/ml exogenous hr-activin A; and the effect of activin A alone in the assay (open circles). Each point represents the mean and standard deviation of n=3 assays. The dotted line represents the limit of detection of the assay.

FIG. 6, consisting of parts A-L, shows localization of activin β_{C} subunit, high molecular weight (HMW) cytokeratins and α smooth muscle actin in the developing rat ventral prostate lobes at day 0 (A, B), 2 (C, D), 4 (E, F), 8 (G, H) and 15 (I, J, K, L). Activin pc subunit immunolocalization (brown staining) shown in A, C, E, G, I, and K and high molecular weight (HMW) cytokeratins (brown staining) and α smooth muscle actin (purple staining) shown in B, D, F, H, J, and L. Immunoreactivity for activin β_{C} subunit was localized to the solid epithelial buds on days 0-4 (A, C, E) which were positive for HMW cytokeratin (B, D, F). At day 8, activin β_{C} subunit immunoreactivity was also observed in the epithelial cells of more mature canalising ducts (G). Activin β_{C} subunit protein was also immunolocalized to smooth muscle cells from day 2-8, which was identified by α smooth muscle actin immunoreactivity (B, D, F, H). At day 15 strong activin β_{C} immunoreactivity was observed in columnar epithelial cells (I, K) and smooth muscle sheaths (K), as identified with (x smooth muscle actin (L). Activin β_{C} immunoreactivity was also observed in fibroblastic stroma from day 4-15.

FIG. 7 shows cross-reactivity of purified clone β_{C} antibody with β_{A}, β_{B}, β_{C}, and β_{E} peptides using ELISA. Graph demonstrates the dilution factor of the β_{C} antibody reacted against 1 mg/ml β_{A}, β_{B}, β_{C}, and β_{E} peptide or uncoated control. Absorbance decreased with decreasing concentrations of β_{C} antibody. Clone 1 β_{C} antibody showed minimal cross-reactivity (0.1%) with β_{A}, β_{B} or β_{E} peptides by ELISA, as shown in FIG. 7.

FIG. 8, consisting of photographs A, B and C, shows immunolocalization of β_{C} subunit protein to human liver. βc-subunit immunoreactivity was localized to hepatocytes using the β_{C} clone 1 supernatant (arrow, A) and purified clone 1 antibody (B). Specificity of staining was shown by preabsorption with β_{C} synthetic peptide, which abolished immunostaining (C)

FIG. 9, consisting of photographs A-W, shows localization of β_{C} subunit protein relative to that of β_{A} and β_{B} subunits was compared in tissue from patients with Benign Prostatic Hyperplasia (BPH) (FIG. 9). As previously reported, β_{A} subunit was localized to the basal (←) and secretory ( [symbol] ) epithelial cells (A), whereas β_{B} subunit was localized to the basal epithelial cells only (B). β_{C} subunit immunoreactivity was present in basal epithelial cells (C). No immunoreactivity was detected when the β_{C} antibody was preabsorbed with β_{C} peptide (D). Localization of β_{C} subunit relative to β_{A} and β_{B} subunits in tumour tissue from patients with high grade cancer is shown in FIG. 4. In 10 patients with poorly differentiated prostate cancer, immunoreactivity for β_{A} (E), β_{B} (F), and β_{c} (G) subunits was detected in tumour cells in all patients. Preabsorption of β_{C antibody with β}_{C} peptide abolished staining (H). These patterns of staining suggested that the same cell types contain β_{A}, β_{B}, and β_{C}, and to expand this further, serial tissue sections from BPH patients were used. All β_{A}, β_{B}, and β_{C} subunits were colocalized to basal epithelial cells (I, J, and K, respectively). Because the total thickness of the three serial sections examined was large (0.9 mm) relative to the cell diameter, the boundary pattern of the cells within the focus plane appeared different. In addition, activin β_{B} was localized to stromal cells (L), which were identified as a subset of smooth muscle cells (M). Stromal staining for activin β_{C} (N) was localized to a subset of smooth muscle cells in the stroma (O), therefore β_{B} and β_{C} colocalized in α-actin-positive stroma. In serial tissue sections, β_{A} (P) and β_{C} (R) subunit proteins were localized to nerve cells, which were identified by neurofilament immunoreactivity (S). No immunoreactivity for β_{B}-subunit protein (O) or control mouse IgG (inset) was detected. Using serial sections of prostate tissue, blood vessel smooth muscle was identified by α-smooth muscle actin staining (W). β_{A} (T), β_{B} (U), and β_{C} (V) activin subunits were localized to the cells of blood vessels. No immunoreactivity was detected in the control section (inset).

FIG. 10, consisting of photographs A and B, shows formation of activins comprised of β_{A}, β_{B} and β_{C} subunit proteins. Autoradiograph of supernatants from transfected and ³⁵S-labeled 293 cells run under nonreducing conditions on a 12% polyacrylamide gel (A). Cells transfected with β_{A} alone produced approximately 24-kDa activin β_{A}-β_{A} complexes. A 43 to 46 kDa high molecular mass band corresponds to the pro-β_{A} protein (lane 1). Cells transfected with β_{B} alone produced approximately 22-kDa activin β_{B}-β_{B} complexes (lane 2). Transfection of β_{C} alone produced approximately 20 kDa activin β_{C}-β_{C} complexes (lane 3). Cells cotransfected with β_{A} and β_{C} subunits produced an activin dimer β_{A}-β_{C} of about 23 kDa. A significant amount of β_{A}-β_{A} complexes were also formed; β_{C}-β_{c} was formed in low amounts (lane 4). Cells cotransfected with β_{B} and/β_{C} subunits produced activin dimer β_{B}-β_{C} complexes of about 21 kDa, β_{B}-β_{B} complexes were also formed in a higher amount compared with β_{C}-β_{C} complexes (lane 5). Cells cotransfected with α and β_{A} subunits produced pro-β_{A}, both mono- (30 kDa) and diglycosylated (32 kDa) forms of β-PA complexes (*), and pro-αC and β_{A}-β_{A} complexes (lane 6). Cells cotransfected with a and β_{B} subunits produced both mono-(29 kDa) and diglycosylated (31 kDa) forms of 1-β_{B} complexes (*) and pro-α_{C} and β_{B}-β_{B} complexes (lane 7). Cells cotransfected with α and β_{C} subunits produced only β_{C}-β_{C} complexes (lane 8). Control lanes consisted of cells transfected with a alone (lane 9), the pRK5 control plasmid alone (lane 10), and pro-α inhibin subunit (lane 11). (B) Analysis of inhibin α and β dimers by immunoprecipitation. Supernatants from transfected and ³⁵S-labeled cells were immunoprecipitated using αC subunit antiserum 29A and analyzed on a 10% SDS-PAGE gel under nonreducing conditions. Cells cotransfected with the a and β_{A} subunits produced mono- and diglycosylated α-β_{A} with molecular masses of approximately 30 and 32 kDa, respectively. High molecular mass bands of pro-αN-αC-β_{A} (60 kDa) and AN-αC-β_{A} (55 kDa) were also formed (lane 1). Cells cotransfected with α and β_{B} subunits produced mono- and diglycosylated α-β_{B} with molecular massess of 29 and 31 kDa, respectively (lane 2). Cells transfected with α and β_{C} subunits did not produce any α-subunit-containing complexes (lane 3). Control lanes consisted of cells transfected with the α-subunit (lane 4) and pRK5-transfected cells (lane 5).

FIG. 11, consisting of graphs A and B, shows the effect of activin A and activin C, alone or in combination, on DNA synthesis by LNCaP and HepG2 tumour cells. LNCaP (A) and HepG2 (B) cells were plated and cultured in DMEM and 5% FCS, and the medium was replenished on day 3 with activin A, activin C, or a combination of these treatments. Activin A (40 ng/ml; A) or activin C (40 or 200 ng/ml) and matching vehicle control buffers were added alone. Activin C (40 or 200 ng/ml) or matching vehicle buffer controls were added 1 h before addition of activin A. Each value represents the mean±SD from five replicate wells. *Significance between P<0.001 and P<0.006.

FIG. 12, consisting of parts A-O, shows immunolocalisation of activin β_{C} subunit protein in human and bovine endocrine organs. Insets show low power view of whole tissue section.

(A) Activin β_{C} subunit protein was localised to the stromal tissue (arrow) of the human ovary. However, it should be noted that this tissue section did not contain follicles, therefore refer to bovine ovary as reported in (D-H) for follicular pattern of staining. (B) Strong nuclear (arrow) and cytoplasmic staining (arrowhead) was observed in tissue from a patient with an ovarian endodermal sinus tumour. (C) Cytoplasmic staining (arrowhead) was predominant in an ovarian mucinous adenocarcinoma patient, while nuclear localisation (arrow) was observed to a lesser degree.

The bovine ovary displays a distinct pattern of activin β_{C} subunit protein immunolocalisation in both the ovarian stroma and follicles. (D) Strong stromal staining is observed, however the primoridal follicle is negative (arrow). (E) A pre-antral follicle has weak positive immunolocalisation (arrow), and is surrounded by strong stromal staining. (F) In an antral follicle, thecal cells (arrow), granulosa cells (arrowhead) and cumulus cells (asterisk) immunolocalise activin β_{C} subunit protein. (G) The corpus luteum (arrow) displays strong staining for the activin β_{C} subunit. (H) The smooth muscle of the vasculature is positive for activin β_{C} subunit protein, as are the surrounding stromal cells. (I) In the testis of a normal male, all spermatogenic cells (i.e. spermatogonia and spermatids) displayed activin β_{C} subunit protein localisation. However, staining was absent in spermatozoa. Some nuclear localisation was also observed (arrow). Leydig cells immunolocalise activin β_{C} subunit protein. (J) Activin β_{C} subunit protein cytoplasmic (arrowhead) and nuclear staining (arrow) was observed in a patient with testicular seminoma. (K) The cortex of the adrenal gland displays an isolated nuclear (arrow) staining pattern for activin β_{C} subunit protein. Weak positive and strong postive (arrowhead) cytoplasmic staining was also observed in the adrenal medulla. (L) Tissue from a patient with adrenal cortical carcinoma displayed predominantly strong nuclear (arrow) activin β_{C} subunit immunolocalisation, however cytoplasmic (arrowhead) staining was also observed. (M) The follicles of the thyroid gland display intermittent immunolocalisation for activin β_{C} subunit protein. Predominantly, epithelial cells of the follicles display no staining (arrow) for the activin β_{C} subunit, however some epithelial cells have cytoplasmic localisation (arrowhead). (N) In contrast, a patient with thyroid minimally invasive follicular carcinoma displayed strong activin β_{C} subunit staining in the cytoplasm (arrow). (O) In addition, a patient with papillary carcinoma of the thyroid gland immunolocalised strongly to the nuclei (arrow) and was less intense in the cytoplasm (arrowhead).

FIG. 13, consisting of parts A-I, shows immunolocalisation of activin β_{C} subunit protein in normal human digestive tissues and following the development of adenocarcimoma. Insets show low power view of whole tissue section.

(A,B) Activin β_{C} subunit protein was localised to epithelial cells (arrow) of the human stomach, however the staining pattern was variable. Smooth muscle cells and macrophages displayed variable staining. (C) In a patient with moderately differentiated stomach adenocarcinoma, a pattern of predominantly cytoplasmic staining (arrow) was observed. (D) In contrast, a patient with poorly differentiated stomach adenocarcinoma displayed strong nuclear (arrow) staining, with less intense cytoplasmic (arrowhead) staining. (E) Similarly, in patients with stomach adenocarcinoma that metastasised to the lymph node, strong nuclear staining (arrow) was observed. (F) The benign colon displays strong activin β_{C} subunit protein immunolocalisation in some secretory epithelial cells (arrow) and smooth muscle cells. Nuclear staining was observed intermittently (G) Tissue from a patient with adenocarcioma of the colon displayed strong nuclear (arrow) and cytoplasmic (arrowhead) staining. (H) The normal rectum displayed both cytoplasmsic and nuclear staining of the surface epithelium (I) Rectal adenocarcinoma displayed both nuclear (arrow) and cytoplasmic (arrowhead) staining however this is not observed in all tumour cells.

FIG. 14, consisting of parts A-I, shows immunolocalisation of activin β_{C} subunit protein in normal human lung, urinary bladder, endometrium, ovary and following the development of adenocarcinoma in these tissues. Insets show low power view of whole tissue section.

(A) The alveolar epithelium of the normal lung does not immunolocalise the activin β_{C} subunit, however the stromal cells surrounding these alveolar cells have positive staining. (B) Tissue from a patient with adenocarcinoma of the lung, reveals predominant nuclear staining and weaker cytoplasmic staining. (C) The transitional epithelium of the urinary bladder immunolocalises activin β_{C} subunit protein, both the cytoplasm and some nuclei. Intermittent smooth muscle cells also display positive staining. (D) Urinary bladder poorly differentiated carcinoma strongly immunolocalises the nuclei of these tumour cells, however the cytoplasm also shows positive staining. (E) The proliferative glands of the endometrium strongly immunolocalise the activin β_{C} subunit. (F) In contrast the secretory glands display weaker staining. (G) Similarly to the benign proliferative phase, tissue from endometrial adenocarcinoma patients display a strong staining pattern for activin β_{C} subunit protein in both the nuclei and cytoplasm of these tumour cells. (H) An example of benign human ovary strongly immunolocalising activin β_{C} subunit protein in stromal tissue. (I) Tissue from a patient with ovarian mucinous adenocarcinoma shows an intense staining pattern in the cytoplasm and nucleus of these tumour cells.

FIG. 15, consisting of parts A-K, shows immunolocalisation of activin β_{C} subunit protein in normal human lung, skin, breast, lymph node and following the development of cancer in these tissues. Inserts show low power view of whole tissue section.

(A) The tissue of the normal lung displays activin β_{C} subunit immunolocalisation in the cytoplasm of the stromal cells surrounding the alveolar epithelium, which are negative. (B) In contrast, the tumour cells from patients with lung adenocarcinoma strongly immunolocalise activin PC subunit protein in the nuclei and more weakly in the cytoplasm. (C) In addition, tissue from a patient with lung squamous cell carcinoma also displayed strong nuclear and cytoplasmic staining. (D) The skin immunolocalises the activin β_{C} subunit in the cytoplasm of keratinocytes as well as some nuclei, hair follicles, and blood vessels. (E) In tissue from a patient with skin squamous cell carcinoma, activin β_{C} subunit protein strongly immunolocalises to the nuclei of the tumour cells, however the cytoplasm is also postive. (F) Normal breast epithelium immunolocalises activin β_{C} subunit protein. Myoepithelial cells displayed both positive (arrow) and negative staining (arrowhead), however the secretory epithelial cells showed strong cytoplasmic localisation (asterisk). (G) In contrast, patients with breast residual infiltrating duct carcinoma display strong nuclear staining, as well as cytoplasmic localisation. (H) Breast infiltrating lobular carcinoma tissue also displayed predominantly nuclear localisation associated with weak cytoplasmic staining. (I) Tissue from a patient with breast papillary carcinoma displayed strong nuclear and cytoplasmic staining. (J) The normal lymph node tissue immunolocalised activin β_{C} subunit protein in the stromal tissue (arrow) surrounding the lymphyocytes. However the lymphocytes themselves were negative for the activin β_{C} subunit (arrowhead). (K) Tissue from a patient with lymphoma displayed strong nuclear staining, however not all nuclei were positive. Some tumour cells displayed cytoplasmic immunolocalisation.

FIG. 16, consisting of parts A-H, shows immunolocalisation of activin β_{C} subunit protein in normal human salivary gland, bone, nasal cavity and following the development of cancer in these tissues. Insets show low power view of whole tissue section. (A) In the salivary gland, cytoplasmic localisation for activin β_{C} subunit protein is observed in the ducts (arrow), serous cells (arrowhead), mucous cells (asterisk) and nerves of this organ. (B) In a patient a warthin tumour of the parotid gland, cytoplasmic and some nuclei staining is observed in the tumour cells. (C) Tissue from a patient with carcinoma of the submandibular gland immunolocalises activin β_{C} subunit protein to the cytoplasm and the nuclei of these tumour cells. (D) Tissue from a patient with low grade chondrosarcoma, activin β_{C} subunit protein displayed focal nuclear localisation of chondrocytes. (E) In contrast, tissue from a patient with bone osteosarcoma shows predominant positive staining in the cytoplasm, however there is also some nuclear staining. (F) Both strong cytoplasmic and nuclear staining is observed in a patient with bone giant cell tumour. (G) Tissue from the normal nasal cavity displays activin β_{C} subunit immunolocalisation in the epithelium of the nasal mucosa. Specifically in both the basal cells (the proliferative area of the epithelium), and more predominantly localised in the secretory epithelial cells. (H) In tissue from a patient with inverted papilloma of the nasal cavity, cytoplasmic and nuclear localisation was observed in the tumour cells.

FIG. 17, consisting of parts A-H, shows immunolocalisation of activin β_{C} subunit protein in normal human stomach and duodenum and following the development of cancer in these tissues. Insets show low power view of whole tissue section. Normal stomach tissue immunolocalises activin β_{C} subunit protein in both the glands and smooth muscle, however this localisation is intermittent with both positive and negative staining. (A) In normal tissue, glands displayed both nu/clear and cytoplasmic immunolocalisation but staining was non-uniform. (B) In the antrum of the stomach displays immunolocalisation in both the mucosa and muscle layers, but not all cells are positive. For example, the gastric surface displays cytoplasmic localisation. (C) The duodenum immunolocalises activin β_{C} subunit protein in both the mucosal and smooth muscle cell layer. Not all cell types are positive and localisation is non-uniform. In the luminal surface secretory cells, some cells that display activin β_{C} subunit localisation in the cytoplasm, while others have nuclear staining in the deeper layers of the mucosa. (D) Tissue from a patient with moderately differentiated stomach adenocarcinoma displayed predominantly cytoplasmic activin β_{C} subunit immunolocalisation. (E) In contrast, both nuclear and cytoplasmic immunolocalisation was observed in a patient with poorly differentiated stomach adenocarcinoma. (F) Nuclear staining was also observed in a patient with signet ring cell carcinoma of the stomach, in addition to stromal staining. (G) Tissue from lymphoma of the stomach displayed a similar pattern of staining in the nuclei of tumour cells and stromal cells. (H) Stomach carcinoma that had metastasised to the lymph node, displayed intermittent nuclear, cytoplasmic and stromal localisation.

FIG. 18, consisting of parts A-H, shows immunolocalisation of activin β_{C} subunit protein in normal human gallbladder, urinary bladder, kidney and following the development of cancer in these tissues. Insets show low power view of whole tissue section. (A) In the normal gallbladder, basal and secretory cells localise the activin β_{C} subunit. Both nuclear and cytoplasmic staining is observed in the epithelial cell layer. Smooth muscle localisation was also observed. (B) Similarly, tissue from a patient with adenocarcinoma of the gallbladder displayed both nuclear and cytoplasmic staining in the tumour cells. In addition, smooth muscle (asterick; inset) in the vicinity of the tumour cells displayed strong activin β_{C} subunit protein localisation. (C) In tissue from the urinary bladder, the transitional epithelium immunolocalises activin β_{C} subunit protein, in a predominantly a cytoplasmic pattern, however some cells do display nuclear immunolocalisation. (D) Tissue from a patient with high grade transitional cell carcinoma of the urinary bladder, immunolocalises activin β_{C} subunit protein in a both cytoplasmic and nuclear pattern in these tumour cells. (E) In addition, poorly differentiated carcinoma cells have strong cytoplasmic and strong nuclear staining. (F) In the cortex of the kidney, the proximal region which is highly metabolic displays positive staining for activin β_{C} subunit protein. This staining is predominantly cytoplasmic, however some cells have nuclear localisation. The glomeruli (asterisk) do not immunolocalise the activin β_{C} subunit. (G) In contrast, the collecting ducts of the medulla of the kidney, have cytoplasmic but not nuclear localisation. (H) Tissue from a patient with transitional carcinoma of the kidney displayed strong localisation of the activin β_{C} subunit in the tumour cells cytoplasm. In addition some tumour cell nuclei displayed positive staining.

FIG. 19, consisting of parts A-H, shows immunolocalisation of activin β_{C} subunit protein in normal human endocrine and reproductive organs; testis, ovary, adrenal gland, uterine cervix and following the development of cancer in these tissues. Insets show low power view of whole tissue sections.

(A) In the normal testis, activin β_{C} subunit protein is localised in both the cytoplasm and some nulcei of spermatogenic cells. (B) Tissue from a patient with testicular seminoma displays strong cytoplasmic and nuclear localisation. (C) The human ovary strongly immunolocalises the activin β_{C} subunit in stromal tissue. (D) Ovarian endodermal sinus tumour cells display strong localisation in the cytoplasm and some nuclei.

(E) In the cortex of the adrenal gland, activin β_{C} subunit protein is observed in the cytoplasm, however this localisation is variable with both weak and strong areas of staining. In addition, nuclear localisation in occasionally observed. (F) Tissue from a patient with cortical carcinoma of the adrenal gland displays strong cytoplasmic and nuclear staining. (G) The uterine cervix displays some nuclear staining, however not all cells are positive. Both the cytoplasm (arrowhead) and nuclei (arrow) immunolocalise the activin β_{C} subunit in squamous dysplasia. (H) Tissue from a patient with squamous cell carcinoma of the uterine cervix immunolocalises the activin β_{C} subunit protein in the cytoplasm (arrowhead) of tumour cells. Some tumour cells also display prominent nuclear (arrow) localisation.

FIG. 20, consisting of parts A-J, shows immunolocalisation of activin β_{C} subunit protein in normal human liver, pancreas, esophagus and following the development of cancer in these tissues. Insets show low power view of whole tissue sections.

(A, B, C) The cytoplasm of hepatocytes in normal liver tissue localise the activin β_{C} subunit. Bile ducts also display positive staining. (D) In tissue from a patient with liver cholangiocarcinoma, cytoplasmic and sporadic nuclear localisation is observed. (E) A patient with hepatocellular carcinoma also displays strong cytoplasmic staining. Some tumour cells also display strong nuclear localisation. (F) Tissue from a patient with gastric cancer, that has metastasised to the liver, immunolocalises activin β_{C} subunit protein in the cytoplasm of the tumour cells. (G) The pancreas immunolocalises activin β_{C} subunit protein strongly in the secretory granules of the acinar cells (arrowhead) and more weakly to the islet cells (arrow). (H) Tissue from a patient with pancreatic cancer displayed stronger activin β_{C} subunit localisation in the tumour cells. Both cytoplasmic and nuclear staining was observed in the tumour cells. (I) In the esophagus, activin pc subunit immunolocalisation was observed in blood vessels and some smooth muscle. However, apart from some sporadic nuclear positive cells, the epithelial layer was negative. (J) Tissue from a patient with squamous cell carcinoma, strongly localised activin β_{C} subunit protein in the cytoplasm of the tumour cells.

FIG. 21, consisting of parts A-H, shows immunolocalisation of activin β_{c} subunit protein in normal human kidney, thyroid, thymus and following the development of cancer in these tissues. Insets show low power view of whole tissue sections.

(A) In the cortex of the kidney, strong activin β_{C} subunit cytoplasmic localisation is observed, however some cells have nuclear localisation. The glomerulus (asterisk) was negative for the activin β_{C} subunit. (B) In tissue from a patient with renal cell carcinoma, strong cytoplasmic localisation is also observed in these patients. (C) In the adrenal gland cortex, both strong and weak cytoplasmic localisation is observed. Some cells also display nuclear localisation. (D) In patients with a neuroendocrine pheochromocytoma (a tumour of the medulla), the tumour cells strongly localise activin β_{C} subunit protein in the cytoplasm. Nuclear staining is sporadic. (E) In the normal thyroid gland, activin β_{C} subunit protein localisation in the epithelial cells of the thyroid follicles is intermittent and the gland is predominantly negative. The positive cells may have both cytoplamsic and nuclear staining. (F) In contrast, tissue from a patient with minimally invasive follicular carcinoma of the thyroid displayed strong localisation in the cytoplasm of the tumour cells. (G) In the normal thymus, lymphocytes are negative for the activin β_{C} subunit (arrowhead), however the thymic epithelium (arrow) displays cytoplasmic and weak nuclear staining. Stromal cells (asterisk) are also positive. (H) In tissue from a patient with thymoma, the tumor cells display strong activin β_{C} subunit protein cytoplasmic localisation. The lymphocytes remain negative for activin β_{C} subunit protein with malignancy.

FIG. 22, consisting of parts A-G, shows immunolocalisation of activin β_{C} subunit protein in normal human myometrium, fallopian tube, placenta and placental cord and in the benign uterus and ovary. Insets show low power view of whole tissue sections.

(A) In the myometrium, activin β_{C} subunit protein immunolocalisation is weak or negative. (B) Tissue from a patient with leiomyoma of the uterus displayed positive staining in smooth muscle cells. Some nuclear staining was also observed. (C) The fallopian tube immunolocalised activin β_{C} subunit protein in secretory cells, some intermittent nuclear staining was also present. (D) Tissue from a patient with fibrothecoma of the ovary, displayed both nuclear and strong cytoplasmic staining. Mature (E) and mid-trimester (F) placental villi immunolocalised activin β_{C} subunit protein in the choronic villi and blood vessels. (G) Umbilical cord displays activin β_{C} subunit localisation in smooth muscle cells.

FIG. 23, consisting of parts A-E, shows immunolocalisation of activin β_{C} subunit protein in normal human tonsil, spleen, heart, appendix and seminal vesicle. Insets show low power view of whole tissue sections.

(A) In the tonsil, activin β_{C} subunit protein localised to the stromal cells (arrow) but not the lymphocytes (arrowhead). (B) In the spleen, blood vessels are strongly positive (arrow), while the lymphoid aggregations (arrowhead) are negative. (C) Heart cardiac muscle (arrow) and nerves (arrowhead) immunolocalise activin β_{C} subunit protein, however the blood vessels were negative. (D) The cytoplasm of the secretory epithelial cells (arrowhead) in the appendix strongly immunolocalise activin β_{C} subunit protein, however some nuclear staining (arrow) is also observed. (E) The secretory epithelial cells of the seminal vesicle displayed both cytoplasmic (arrowhead) and nuclear (arrow) staining for the activin β_{C} subunit. Smooth muscle cells were also positive.

FIG. 24, consisting of parts A-H, shows immunolocalisation of activin β_{C} subunit protein in the normal and diseased human brain. Insets show low power view of whole tissue sections.

(A) In tissue from a patient with glioblastoma, the benign region displays astroctyes that strongly immunolocalise activin β_{C} subunit protein in the cytoplasm (arrow). Reactive astrocytes are also positive. (B) In the same patient, the blood brain barrier (arrow) also strongly localises the activin β_{C} subunit. (C) The cytoplasm of glioblastoma tumour cells (arrow) are positive for activin β_{C} subunit protein. (D) Tissue from a patient with meningioma also strongly localises activin β_{C} subunit protein in the cytoplasm of the tumour cells. (E) The grey matter of the human brain displays positive staining in neuronal cells. Activin β_{C} subunit protein immunolocalises to the white matter (F), the cerebellum (G) and the pituitary gland (H) of the human brain.

FIG. 25, consisting of parts A-E, shows immunolocalisation of activin β_{C} subunit protein in the normal brain of the sheep and both wild type and transgenic mice that express a human Cu,Zn Superoxide Dismutase mutation resulting in neruodegenerative disease.

Both the transgenic mice brain (A) and wild type (B) mouse brain display activin β_{C} subunit localisation in cerebellum. The molecular layer strongly displays activin β_{C} subunit protein (arrow), the granular layer displays less staining (asterisk) and the Purkinje cells (arrowhead) are negative. (C) The endocrine cells (arrow) of the sheep pituitary gland immunolocalise activin β_{C} subunit protein. (D) In the pre-optic area of the sheep brain, neuronal cells with axon processes (arrow) localise the activin β_{C} subunit. (E) In the sheep hypothalamus neuronal cells (arrow) display activin β_{C} subunit protein localisation.

FIG. 26, consisting of parts A-C, shows immunolocalisation of activin β_{C} subunit protein in the benign and malignant human prostate. (A) In tissue from a patient with prostate cancer, activin β_{C} subunit protein immunolocalises strongly to smooth muscle cells (arrow) and basal cells (arrowhead) in the stromal region. (B) In addition, the nerves (arrow) immunolocalise the activin β_{C} subunit. (C) Activin β_{C} subunit protein immunolocalises in prostate tumour cells (arrow).

FIG. 27, consisting of parts A-H, shows immunolocalisation of activin β_{C} subunit and TGF-β protein in serial tissue sections of the normal day 15 rat prostate and malignant human prostate.

(A) Activin β_{C} subunit protein localises to the basal and secretory epithelial cells (arrowhead) and smooth muscle cells (arrow) in the ventral rat prostate. (B) The accompanying serial section to A, displays TGF-β1 protein localisation in smooth muscle cells (arrow). (C) Multilayer smooth muscle cells are evident in the proximal region of the rat prostate as identified with α-actin marker (arrow). (D) The accompanying serial section to C, identifies differential activin β_{C} subunit localisation, with either strong (arrow head) or absent (asterisk) staining. (E) In the proximal region of the rat ventral prostate, activin β_{C} subunit protein localises to the epithelial compartment (arrowhead) and smooth muscle cells (arrow). (F) The accompanying serial section to E, displays TGF-β1 protein localisation in smooth muscle cells (arrow). (G) In tissue from a patient with prostate cancer, activin β_{C} subunit protein localises to prostate tumour cells (arrow). (H) The accompanying serial section to G, displays TGF-β1 protein localisation in the prostate tumour cells (arrow).

FIG. 28, consisting of parts A-H, shows immunolocalisation of activin β_{C} subunit protein in malignant human skin, larynx, tongue, lung, small intestine and disorders of the appendix and soft tissue.

(A) Tissue from a patient with melanoma displays activin β_{C} subunit localisation in the cytoplasm and nuclei of tumour cells. (B) In a patient with pseudomyxoma of the appendix, cytoplasmic and some nuclear staining is observed. (C) Activin β_{C} subunit protein immunolocalises to the cytoplasm and some nuclei in a patient with neurofibromatosis of the soft tissue. (D) Tissue from a patient with squamous cell carcimoma of the larynx displays ctyoplasmic and some nuclear staining. (E) Similarly, squamous cell carcimoma of the tongue immunolocalises activin β_{C} subunit protein in the cytoplasm with some focal nuclear staining. (F) Tumour cells in a patient with small cell carcinoma of the lung display cytoplasmic localisation. (G) In the normal small intestine, non-uniform activin β_{C} subunit localisation was observed in the epithelial cells. (H) Tissue from a patient with malignant stromal tumour of the small intestine displayed strong activin β_{C} subunit protein localisation.

### DESCRIPTION OF THE INVENTION

In a first aspect of the invention there is provided a method of modulating the formation of an activin dimer in a cell or biological sample, the method including controlling levels and bioactivity of activin β_{C} in the cell or biological sample. Preferably, the method includes modulating the formation of activin dimers formed by the dimerisation of activin subunits selected from the group consisting of β_{A}, β_{B}, β_{C}, β_{D} or β_{E}, or combinations thereof.

The present applicants have advantageously found that activin β_{C} subunit can dimerise with activin subunits, such as β_{A}, β_{B} or β_{C} subunits, to inhibit the formation of activin dimers, such as activin A, B or AB thereby modulating the biological activity of these ligands.

The term “activin β_{C}” as used herein includes full length activin β_{C} subunit protein, an active portion thereof, or an activin β_{C} subunit variant that is capable of dimerising with another activin subunit, such as activin β_{A}, β_{B}, β_{C}, β_{D} or β_{E}. Preferably, the activin β_{C} is capable of dimerising with activin β_{A} subunit to form activin heterodimer AC. An activin β_{C} variant may include activin β_{C} which has been modified at the nucleotide or amino acid level and may include additions or deletions or replacements of nucleotides or amino acids which do not affect the functionality of the protein. Activin β_{C} may be natural or recombinant and therefore may be induced to be expressed in a cell or biological sample. The activin β_{C} may be from any animal species, preferably the activin β_{C} is encoded by mammalian DNA, more preferably the activin β_{C} is human, mouse or rat activin β_{C}.

Activin β_{C} has a structure similar to other activins and other members of the TGFβ superfamily. The structure of activins are based on the conservation of the number and spacing of the cysteines within each subunit and the disulphide linkages between the two subunits that form characteristic cysteine knots. Other similarities relate to dimer formation, the location of the bioactive peptide in the carboxy terminal region of the precursor activin subunit molecule and similar intracellular signalling mechanisms. Human activin β_{C}, in comparison with other TGF-β superfamily members, reveals a typical structure with 9 conserved cysteines and a large precursor molecule that contain a core of hydrophobic amino acids at the N terminus thought to be the secretion signal sequence (6). The mouse activin β_{C} also contains 9 conserved cysteines, and N terminal hydrophobic amino acids that may serve as a signal peptide (18).

Activin β_{C} may be obtained from methods of producing monomeric and dimeric activin β_{C} in CHO cells (Biopharm GmbH, Heidelberg, Germany), bacterial cells or mammalian cells. Activin β_{C} monomer and dimer can also be obtained from methods involving insect larvae infected with recombinant baculovirus (19).

The term “modulating” includes inhibiting or inducing the formation of an activin dimer in a cell or biological sample. The method includes controlling levels or bioactivity of activin β_{C} in the cell or biological sample. The phrase “formation of an activin dimer” is taken to mean that at least two activin subunits are dimerised to form an activin heterodimer or homodimer. Preferably, the activin dimer formed is selected from the group consisting of activin AC, activin BC, activin CC, activin DC or activin EC. Alternatively, the activin dimer that may be inhibited from forming is selected from the group consisting of activin A, activin B, activin D, activin E or combinations of heterodimers thereof.

The method preferably includes modulating the formation of activin homodimers selected from the group consisting of activin A (β_{A}-β_{A}), activin B (β_{B}-β_{B}), activin C (β_{C}-β_{C}), activin D (β_{D}-β_{D}) or activin E (β_{E}-β_{E}). The method may preferably include modulating the formation of activin heterodimers selected from the group consisting of activin AB (β_{A}-β_{B}), activin AC (β_{A}-β_{C}), activin AD (β_{A}-β_{D}), activin AE (β_{A}-β_{E}), activin BC (β_{B}-β_{C}), activin BD (β_{B}-β_{D}), activin BE (β_{B}-β_{E}), activin CD (β_{C}-β_{D}), activin CE (β_{C}-β_{E}) or activin ED (β_{E}-β_{D}). Most preferably, the method includes modulating the formation of activin A, activin B or activin C.

The formation of an activin dimer in a cell or biological sample can be detected by general methods of assaying for the specific activin dimer forms. Such assays preferably utilise an antibody that recognises an epitope of an activin subunit. Suitable assays for detecting activin dimer formation may preferably include ELISA, immunohistochemistry, immunoprecipitation, immunoaffinity purification or Western Blot techniques.

In the present invention the formation of activin dimers formed by activin subunits selected from the group consisting of β_{A}, β_{B}, β_{C}, β_{D} or β_{E}, may be regulated by controlling levels or bioactivity of activin β_{c}. The phrase “controlling levels or bioactivity of activin β_{C}” as used herein includes treating a cell or biological sample to modify or alter the level of activin β_{C}, the level of expression and/or activity of activin β_{C}, compared to an untreated cell or biological sample. This may be achieved by treating a cell or biological sample to increase or decrease levels or bioactivity of activin β_{C} in a cell or biological sample. Levels or bioactivity of activin β_{C} may be preferably increased in a cell or biological sample by introducing regulatory factors that increase the expression of activin β_{C} into a cell or biological sample, introducing expression vectors that express activin β_{C} into a cell or biological sample and/or introducing exogenous activin β_{C} into a cell or biological sample. Levels or bioactivity of activin β_{C} may be decreased by introducing antagonists or inhibitory factors that block the expression and/or activity of activin β_{C} in the cell or biological sample.

Methods of controlling levels or bioactivity of activin β_{C} may include methods of directly modifying protein activity, such as but not limited to, dominant negative mutations or chemical moieties generally, and also the use of antibodies specific to activin β_{C}, as discussed later in detail, specific antibodies to a protein that modulates the expression or activity of activin β_{C} or agents that modulate the expression or activity of activin β_{C}.

Dominant negative mutations are mutations to endogenous gene or mutant exogenous genes that when expressed in a cell disrupt the activity of a targeted protein species. In the present application the targeted protein is activin β_{C} protein. A guide to the selection of an appropriate strategy for constructing dominant negative mutations that disrupt activity of a target protein is detailed in Hershkowitz (26).

Levels or bioactivity of activin β_{C} can be increased in a cell by over expressing activin β_{C} protein in the cell. Such over expression can be achieved by, for example, associating a promoter, preferably a controllable or inducible promoter, of increased activity with a nucleotide sequence coding for activin β_{C}.

In addition to dominant negative mutations, mutant activin β_{C} proteins that are sensitive to temperature (or other exogenous factors) can be found by mutagenesis and screening procedures that are well known in the art. Also, one skilled in the art will appreciate that expression of antibodies binding and inhibiting an activin β_{C} can be employed as another dominant negative strategy.

Other suitable methods of controlling activin β_{C} levels or bioactivity may include antisense technology to stop transcription of monomeric activin β_{C} protein; antibody technology to bind to activin β_{C} protein (preferably the antibody needs to be able to be intracellular therefore bind to activin β_{C} before heterodimerisation); or overexpression of activin β_{C} protein with expression vectors or gene therapy.

Methods may be employed to assess levels or bioactivity of activin β_{C}. For instance, “transcript arrays” (also called herein “microarrays”) may be employed to measure levels or bioactivity of activin β_{C} protein in a cell or biological sample. Transcript arrays can be employed for analyzing the transcriptional state in a cell, and especially for measuring the transcriptional states of cells exposed to treatment to increase or decrease levels or bioactivity of activin β_{C}. Transcript arrays may be produced by hybridizing detectably labeled polynucleotides representing the mRNA transcripts present in a cell (e.g., fluorescently labeled cDNA synthesized from total cell mRNA) to a microarray.

Activin β_{C} subunit protein or activin β_{C} subunit antibody may also be utilised in a protein chip, protein array or antibody array. Whereby activin β_{C} subunit protein/antibody are immobilised on a membrane, used to recognise and capture specific antigens, or antigen-associated proteins. The proteins captured on the array can then be detected and analysed. Tissue arrays may also be utilised in the method as herein before described.

In the specification the term “cell(s)” is taken to include any cells. Preferably, the cells are derived from a mammalian species, such as, but not limited to, human, mouse, bovine, sheep or other domestic animals. It is preferred that the cells are selected from the group including, but not limited to, normal, cancer or tumour cells of the prostate, fibroblast, epidermal, placental, ovary, testis, adrenal, brain and neural tissue, liver, kidney, pancreas, heart, neural, thyroid, stomach, colon, lung, urinary bladder, endometrium, breast, lymph node, skin, salivary gland, bone, nasal cavity, duodenum, gallbladder, uterine cervix, thymus, placenta, fallopian tube, uterus, tonsil, spleen, appendix, seminal vesicle, larynx, tongue, small intestine, pituitary, rectum, esophagus, myometrium and soft tissue or muscle cells. The cells may be normal cells, diseased cells, adult cells or embryonic cells.

The cells may be single cells, cultured cells or part of a tissue. The cells may be genetically modified recombinant cells, such as transgenic cells. Preferably, the cells express activin pc. The cells may be part of a whole animal thereby providing an in vivo modulation of the formation of an activin dimer in a cell. The cells may also be derived from a cell line. Preferably, the cells are derived from cell lines derived from, but not limited to, prostate, liver, testis, adrenal, brain and neural tissues, ovary, pancreas, kidney, heart, reproductive organs, skeletal muscle, adrenal gland, thyroid gland, stomach, colon, lung, urinary bladder, endometrium, breast, lymph node, skin, salivary gland, bone, nasal cavity, duodenum, gallbladder, uterine cervix, thymus, placenta, fallopian tube, uterus, tonsil, spleen, appendix, seminal vesicle, larynx, tongue, small intestine, soft tissue, rectum, esophagus, myometrium and pituitary cells. More preferably, the cell lines are selected from the group including human prostate tumour cell lines LNCaP, DU145 or PC3, human liver cell line HepG2, CHO ovary cell line or human embryonic kidney 293T. It is preferred that the cells suitable in the methods of the present invention are prostate cells. More preferably, the cells are human prostate cells that may include tumour cells. The cells may be from human prostate cancer patients or liver disease patients.

In the specification the term “biological sample” is taken to include, but not be limited to, serum, tissue extracts, body fluids, cell culture medium, extracellular medium, supernatants, biopsy specimens or resected tissue. The biological sample may include cells as described earlier. Preferably, the biological sample is derived from a mammalian organism, most preferably a human subject. More preferably, the biological sample is, but not limited to, human prostate tissue, ovarian follicular fluid, conditioned media of prostate cells, such as PC3 cells, human serum, seminal fluid or seminal plasma.

In a preferred aspect of the invention there is provided a method of inhibiting the formation of an activin dimer in a cell or biological sample, the method including increasing levels or bioactivity of activin β_{C} in the cell or biological sample.

The term “inhibiting” is taken to mean the formation of an activin dimer in a cell or biological sample is decreased or prevented. In the present invention a cell or biological sample is treated to increase the levels or bioactivity of activin β_{C} in the cell or biological sample to result in the inhibition of the formation of an activin dimer in the cell or biological sample as compared to an untreated cell or biological sample.

The activin dimers that are inhibited from forming are preferably selected from the group consisting of activin A (β_{A}-β_{A}), activin B (β_{B}-β_{B}), activin D (β_{D}-β_{D}), activin E (β_{E}-β_{E}), activin AB (β_{A}-β_{B}), activin AD (β_{A}-β_{D}), activin AE (β_{A}-β_{E}), activin BD (β_{B}-β_{D}), activin BE (β_{B}-β_{E}), or activin ED (β_{E}-β_{D}) Most preferably, the method includes modulating the formation of activin A, activin B or activin C. In the method, activin β_{C} levels or bioactivity are preferably increased by delivering an amount of activin β_{C} in the cell or biological sample or increasing the expression of activin β_{C} in the cell or biological sample.

Levels or bioactivity of activin β_{C} can be increased in a cell or biological sample by preferably delivering an amount of endogenous or exogenous activin β_{C} to the cell or biological sample such that the concentration of activin β_{C} in the cell or biological sample is increased. Activin β_{C} may be obtained from various cellular and animal sources. The activin β_{C} may be naturally purified forms or recombinant forms of the protein.

Alternatively, levels or bioactivity of activin β_{C} may be increased in a cell or biological sample preferably by increasing expression of activin β_{C} in the cell or biological sample. The expression of activin β_{C} can be increased in a cell by introducing regulatory factors into the cell such that the expression of activin β_{C} is increased in the cell.

The levels or bioactivity of activin β_{C} can be preferably increased by providing cellular conditions that favour the expression of activin β_{C}. For instance, the introduction of increased levels or bioactivity of regulatory factors, in a cell may be used to increase the levels or bioactivity of activin β_{C} in a cell or biological sample.

Preferably, the levels or bioactivity of activin β_{C} in a cell or biological sample may be increased by introducing an expression vector including cDNA encoding activin β_{C} in the cell or biological sample. It is preferred that the expression of the cDNA in the expression vector is controlled by an inducible promoter. The expression vector and inducible promoter can be any suitable vector or promoter known to those skilled in the field. More preferably, the cDNA is human, rat or mouse activin β_{C} cDNA which is inserted into a suitable vector. The cDNA sequences may include human—X82540, mouse—NM010565 or Norway rat—AF140031. For example, activin β_{C} cDNA may be preferably subcloned into pRK5 expression vector. The expression vector can be inserted into any cell, such as, but not limited to a prostate cell or liver cell. A vector, for example, HSV may also be used for gene therapy.

In a preferred embodiment exemplified in the examples, PC3 prostate tumour cells, overexpressing activin β_{C} subunit led to a measurable increase of activin AC levels or bioactivity in vitro, a reduction in the levels or bioactivity of activin A and a subsequent decrease in activin signaling as determined by activation of activin response element (ARE). These data demonstrate that activin β_{C} subunit expression antagonized activin β_{A} subunit homodimerisation by forming activin AC heterodimers. It may be possible that activin AC could block activin A from binding to its receptor and therefore from transducing a signal, or activin AC could have its own unique effect.

Preferably, the levels or bioactivity of activin β_{C} in a cell may be increased by producing a transgenic cell that is stably transformed with DNA encoding activin β_{C}_{_{—}}combined with a suitable promoter. The DNA is preferably cDNA encoding full-length activin β_{C}. More preferably, the cDNA is human activin β_{C} cDNA, murine or rat activin β_{C} cDNA.

Transgenic cells that are stably transformed with DNA encoding activin β_{C} may be used to generate cell lines and/or transgenic animals that are genetically engineered to express activin β_{C} or suppress activin β_{C} expression. Preferably, the transgenic animal is a mouse that can be used as an animal model to test activin dimer formation. A transgenic mouse with a prostate specific promoter for activin β_{C} may also be used.

In another preferred aspect of the invention there is provided a method of inducing the formation of an activin dimer in a cell or biological sample, the method including decreasing levels or bioactivity of activin β_{C} in the cell or biological sample.

The phrase “inducing the formation of an activin dimer” as used herein is taken to mean that that at least two activin subunits are brought about to dimerise to form an activin heterodimer or homodimer. Preferably the activin dimers that are induced to form are preferably selected from the group consisting of activin A (β_{A}-β_{A}), activin B (β_{B}-β_{B}), activin D (β_{D}-β_{D}), activin E (β_{D}-β_{E}), activin AB (β_{A}-β_{B}), activin AD (β_{A}-β_{D}), activin AE (β_{A}-β_{E}), activin BD (β_{B}-β_{D}), activin BE (β_{B}-β_{E}), or activin ED (β_{E}-β_{D}). Most preferably, the method includes inducing the formation of activin A, activin B, activin AC or activin C.

The term “inducing” as used herein is taken to mean that a cell or biological sample may be treated to decrease the levels or bioactivity of activin β_{C} to bring about the formation of an activin dimer in the cell or biological sample, as compared to an untreated cell or biological sample.

Preferably, levels or bioactivity of activin β_{C} are decreased by an activin β_{C} inhibitory molecule such as an antibody against activin β_{C}, an activin β_{C} antisense oligonucleotide or an agent that decreases the expression of activin

The activin β_{C} levels or bioactivity may be decreased by an inhibitory molecule or antagonist such as an antibody against activin β_{C}. Blocking antibodies directed against activin β_{C} may be identified by testing antibodies for their ability to inhibit the formation of activin dimers having an activin β_{C} subunit. In the production of antibodies, screening for the desired antibody can be accomplished by techniques known in the art, e.g, ELISA (enzyme-linked immunosorbent assay). To select suitable antibodies specific to activin β_{C}, one may assay generated hybridomas or phage display antibody libraries for an antibody that binds to activin β_{C}.

The term “antibody” as used herein is used in the broadest sense and specifically covers monoclonal antibodies, polyclonal antibodies, multispecific antibodies (e.g., bispecific antibodies), and antibody fragments so long as they bind specifically to a target antigen. Antibodies may be obtained from commercial sources.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method, isolated from phage antibody libraries, or may be made by recombinant DNA methods. The monoclonal antibodies may also be obtained from commercial sources.

Therefore, suitable antibodies specific to activin β_{C} can include, but are not limited to, polyclonal, monoclonal, chimeric, single chain, Fab fragments, and a Fab expression library. For preparation of monoclonal antibodies directed towards activin β_{C} protein, any technique that provides for the production of antibody molecules by continuous cell lines in culture may be used. Such techniques include, but are not restricted to, the hybridoma technique originally developed by Kohler and Milstein (20), the trioma technique, the human B-cell hybridoma technique (21), and the EBV hybridoma technique to produce human monoclonal antibodies (22).

Various procedures known in the art may be used for the production of polyclonal antibodies to an activin β_{C} protein. For production of the antibody, various host animals can be immunized by injection with activin β_{C} protein, such host animals include, but are not limited to, rabbits, mice, rats, etc. Various adjuvants can be used to increase the immunological response, depending on the host species, and include, but are not limited to, Freud's (complete and incomplete), mineral gels such as aluminum hydroxide, surface active substances such as lysolecithin, pluronic polyols, polyanions, peptides, oil emulsions, dinitrophenol, and potentially useful human adjuvants such as bacillus Calmette-Guerin (BCG) and corynebacterium parvum.

Suitable antibodies that specifically bind to activin β_{C} can be introduced into a cell in numerous fashions, including, for example, microinjection of antibodies into a cell (23) or transforming hybridome mRNA encoding a desired antibody into a cell (24).

Suitable inhibitory molecules may include antibody fragments that contain the idiotypes of an activin β_{C} protein. Such antibody fragments can be generated by techniques known in the art. For example, such fragments include, but are not limited to, the F(ab′)₂ fragment which can be produced by pepsin digestion of the antibody molecule; the Fab′ fragments that can be generated by reducing the disulphide bridges of the F(ab′)₂ fragment, the Fab fragments that can be generated by treating the antibody molecule with papain and a reducing agent, and Fv fragments.

In a further technique, recombinant antibodies specific to activin β_{C} protein can be engineered and ectopically expressed in a wide variety of cell types to bind to activin β_{C} as well as to block activin β_{C} from dimerising.

The preparation and use of antibodies according to the present invention may be achieved using techniques well known in the art, and include various antibody labeling techniques and applications. Suitable labels for antibodies include, but are not limited to, radionucleotides, enzymes, substrates, cofactors, inhibitors, fluorescent agents, chemiluminescent agents, magnetic particles and the like. The antibody may also be treated prior to adding the label, for example by biotinylation.

The term “label” when used herein refers to a compound or composition which is conjugated or fused directly or indirectly to a reagent such as an antibody and facilitates detection of the reagent to which it is conjugated or fused. The label itself may be detectable (e.g., radioisotope labels or fluorescent labels) or, in the case of an enzymatic label, may catalyze chemical alteration of a substrate compound or composition which is detectable.

Labeling of the antibody of the present invention may be achieved directly or indirectly. Well known conjugation methods may be used for attaching labels to antibodies. Preferably, after labeling, unbound label is removed from the labeled antibody using purification procedures known to those of skill in the art. The antibody may also be fractionated to provide an immunoglobulin fraction such as IgG or IgM fractions. These antibody fractions may be isolated using methods known to those in the art including using recombinant protein G for IgG or immunoprecipitation for IgM.

Most preferably, a suitable inhibitory molecule is a purified antibody, wherein the antibody recognises an epitope of an activin β_{C} subunit. Preferably, the antibody is capable of recognising monomeric or dimeric forms of activin β_{C} More preferably, the antibody recognises an epitope of activin β_{C} that includes the amino acid sequence VPTARRPLSLLYYDRDSNIVKTDIPDMVVEAC. More preferably the antibody recognises human activin β_{C}.

Levels or bioactivity of activin β_{C} can also be decreased by suppressing expression of activin β_{C}. Suitable antisense oligonucleotide sequences (single stranded DNA fragments) of activin β_{C} may also be used to decrease the levels or bioactivity of activin β_{C}. These may be created or identified by their ability to suppress the expression of activin β_{C}. The production of antisense oligonucleotides for a given protein is described in, for example, Stein and Cohen, 1988 (27) and van der Krol et al., 1988 (28).

Other suitable activin β_{C} inhibitory molecules may include Follistatin (an activin binding protein) which may bind to activin β_{C} and inhibit the function of activin β_{C} or inhibit the dimerisation of activin β_{C} with other activin β subunits. The interplay between activins and the activin-binding proteins, follistatins, regulates ligand bioactivity in many cells and tissues. There are different isoforms of follistatin, FS288 and FS315. Both isoforms bind activin A with similar affinity. FS315 is the predominant form of follistatin in the circulation, whereas FS288 is associated with cell surface heparan-sulphate proteoglycans and plays a role in the inactivation and clearance of the activin ligands. It is also possible that activin β_{C} heteromdimerises with other TGF-β superfamily members to antagonise the actions of these proteins, for example BMPs, TGF-β, or nodal. Antisense technology or antibody technology may be employed intracellularly to prevent either the production of activin β_{C} protein or dimerisation.

In another aspect of the invention there is provided a purified antibody, wherein the antibody recognises an epitope of an activin β_{C} subunit. Preferably, the antibody is capable of recognising monomeric or dimeric forms of activin β_{C}. More preferably, the antibody recognises an epitope of activin β_{C} that includes the amino acid sequence VPTARRPLSLLYYDRDSNIVKTDIPDMVVEAC. It is preferred that the antibody is a monoclonal antibody. Preferably, the antibody is specific to an activin β_{C} subunit. More preferably, the antibody is specific to the human activin β_{C} subunit. The antibody may be a mouse monoclonal antibody developed against the human activin β_{C} subunit. Most preferably, the antibody does not cross react with activin β_{A}, β_{B} or β_{E} peptides. The activin β_{C} antibody of the present invention may be used in a number of methods and diagnostic and/or prognostic techniques. For instance, the activin β_{C} antibody of the present invention may be used in ELISA, immunohistochemistry, immunoaffinity purification, immunoprecipitation, Western Blot and antibody binding studies. Preferably, the activin β_{C} antibody may be used in ELISA methods for diagnostic and/or prognostic purposes, such as diagnosing and/or prognosing activin related diseases. Human and/or animal serum, tissues, fluids, culture supernatants may be used in assays based on activin β_{C} antibody. The activin β_{C} antibody of the present invention may also be used as an inhibitory molecule to inhibit activin β_{C} activity and binding.

In another aspect of the invention there is provided a method of detecting an activin β_{C} subunit and/or an activin dimer including an activin β_{C} subunit, wherein the method includes detecting an activin β_{C} subunit and/or an activin dimer including an activin β_{C} subunit with an antibody that recognises an epitope of an activin β_{C} subunit.

In a preferred aspect of the invention there is provided a method of detecting an activin β_{C} dimer, the method including detecting an activin β_{C} dimer with an antibody that recognises an epitope of an activin β_{C} subunit. Preferably, the activin β_{C} dimer is selected from the group consisting of activin AC (β_{A}-β_{C}), activin BC (β_{B}-β_{C}), activin C (β_{C}-β_{C}), activin CD (β_{C}-β_{D}) or activin CE (β_{C}-β_{E}). Most preferably, the activin β_{C} dimer to be detected is activin AC (β_{A}-β_{C}).

In yet another aspect of the invention there is provided a method of detecting an activin β_{C} dimer in a biological sample, the method including the steps of:
(a) contacting a first antibody that recognises an epitope of a first activin β subunit with a biological sample;
(b) allowing the first antibody to bind to a first activin β subunit in the sample;
(c) washing the sample to substantially remove any unbound material in the sample;
(d) contacting the sample with a second antibody that recognises an epitope of a second activin β subunit, wherein the second antibody is tagged with a labelling agent; and
(e) detecting the labelling agent to identify an activin β_{C} dimer in the biological sample, wherein the first or second antibody recognises an epitope of an activin β_{C} subunit.

Preferably, the activin β_{C} dimer detected is selected from the group consisting of activin AC (β_{A}-β_{C}), activin BC (β_{B}-β_{C}), activin C (β_{C}-β_{C}), activin CD (β_{C}-β_{D}) or activin CE (β_{C}-β_{E}). Most preferably, the activin β_{C} dimer to be detected is activin AC (β_{A}-β_{C}). In the method it is preferred that the first antibody recognises an epitope of an activin β_{C} subunit. Preferably, the second antibody recognises an epitope of an activin β_{A} or β_{B} subunit. More preferably, the second antibody recognises an epitope of an activin β_{A} subunit. Preferably, step (e) includes quantifying the amount of an activin β_{C} dimer in the biological sample.

The biological sample used in the method may included samples as previously discussed. Such samples, may preferably include serum, tissue culture supernatant, seminal plasma, cell lysates, tissue homogenates, biological fluids (ie. Follicular fluid (ovary), interstitial fluid (testes), cerebrospinal fluid, seminal or prostatic fluid (seminal vesicle and prostate). Preferably, the biological sample is from a mammalian animal. More preferably, the biological sample is from a human.

In step (a) of the method a first antibody that recognises an epitope of a first activin subunit is contacted with a biological sample. Preferably, the first antibody is coated on a plate, such as a 96 well plate and the biological sample is added to the coated plate. The biological sample may be added neat or in a diluted form. The first antibody coated on a plate is typically referred to as the “capture antibody”. In the present method it is preferred that the first antibody recognises an epitope of an activin β subunit. Most preferably, the first antibody is a purified antibody is that is capable of recognising monomeric or dimeric forms of activin β_{C}. The antibody preferably recognises an epitope of activin β_{C} that includes the amino acid sequence VPTARRPLSLLYYDRDSNIVKTDIPDMVVEAC Preferably, the antibody is specific to activin β_{C} and more preferably is a monoclonal antibody. The first antibody may be a highly specific activin β_{C} mouse, anti-human or anti-rat antibody.

The biological sample may be pretreated before contacting the sample with the first antibody. For instance, the sample may be diluted with a suitable diluent, such tissue culture media and/or PBS. The sample may preferably be denatured with SDS by heating before contacting the sample with the first antibody. The biological sample may preferably be treated to oxidise the sample. More preferably, activin β_{C} subunit in the sample is oxidised, such that a methionine on an activin β_{C} subunit is oxidised. A suitable oxidising agent, such as H₂O₂ may be added to the biological sample to oxidise the methionine on an activin β_{A} subunit.

In a preferred embodiment, the method includes the additional step of adding a dissociating agent to the sample to remove binding proteins. Preferably, the dissociating agent is added before step (a). Preferably, the binding protein removed is selected from the group consisting of follistatins, BMPs or α-2 macroglobulins. SDS may preferably be added to sample as a dissociating agent to remove binding proteins such as follistatins, BMPs, α-2 macroglobulins and others). However other dissociating agents include those published in McFarlane et al, 1996 (25), which describes sodium deoxycholate, Tween 20, SDS as useful dissociating agents. Binding proteins such as follistatin bind to the β subunits of activin A, B with high affinity, and inhibin A and B with lower affinity. Follistatin may also bind to the activin β_{C} subunit. Therefore, it is preferable to include the dissociating step to remove binding proteins.

In step (b) of the method the first antibody is allowed to bind to a first activin β subunit in the sample. This is preferably achieved by incubating the first antibody and the biological sample under suitable conditions. For instance, suitable media including BSA and/or PBS may be used, preferably activin free serum is used. Most preferably, the sample is incubated over night in a humidifed environment.

In step (c) of the method the sample is washed to substantially remove any unbound material in the sample. The sample is washed in any suitable washing solution, preferably including water or PBS. The sample is preferably washed such that the labelled antibody specifically binds to the target activin subunit.

In step (d) of the method the sample is contacted with a second antibody that recognises an epitope of a second activin β subunit. Preferably, the second antibody recognises an epitope of an activin β_{A}, β_{B}, β_{C}, β_{D} or β_{E} subunit. More preferably, the second antibody recognises an epitope of an activin β_{A} subunit.

The second antibody may be a monoclonal or polyclonal antibody and may be generated by methods previously discussed. The second antibody is required to be tagged with a labelling agent. The preparation and use of antibodies according to the present invention may be achieved using techniques well known in the art, and include various antibody labeling techniques and applications. Suitable labels for antibodies include, but are not limited to, radionucleotides, enzymes, substrates, cofactors, inhibitors, fluorescent agents, chemiluminescent agents, magnetic particles and the like. The antibody may also be treated prior to adding the label, for example by biotinylation.

The term “label” when used herein refers to a compound or composition which is conjugated or fused directly or indirectly to a reagent such as an antibody and facilitates detection of the reagent to which it is conjugated or fused. The label itself may be detectable (e.g., radioisotope labels or fluorescent labels) or, in the case of an enzymatic label, may catalyze chemical alteration of a substrate compound or composition which is detectable. Labelling may include the addition of a subsequent step with a label for example, biotin step, then strepavidin-alkaline phosphatase label.

Labeling of the antibody of the present invention may be achieved directly or indirectly. Well known conjugation methods may be used for attaching labels to antibodies. Preferably, after labelling, unbound label is removed from the labeled antibody using purification procedures known to those of skill in the art. The antibody may also be fractionated to provide an immunoglobulin fraction such as IgG or IgM fractions. These antibody fractions may be isolated using methods known to those in the art including using recombinant protein G for IgG or immunoprecipitation for IgM.

The second antibody that is tagged by a labelling agent as hereinbefore described is typically referred to as the “tag antibody” and is preferably used in a colour detection method. The second antibody may be bound to a labelling agent, such as biotin wherein detection of the label is measured by a coloured enzyme reaction product. Other labelling preferably includes using activin β subunit antibody directly labelled with alkaline phosphatase.

In step (e) of the method an activin dimer that is bound to the second labelled antibody is detected. The method of detection would depend on the labelling agent used to tag the second antibody and then addition of strepavidin alkaline phosphatase. The detection preferably involves colour detection from kit reagents. For instance, colour may be read using a microplate reader using a standard. Calculations on levels or bioactivity of activin AC are based on a standard curve of known amounts of activin AC. For instance, bovine follicular fluid and a human recombinant or purified activin AC protein may be used as a standard for the activin AC assay. Preferably, step (e) includes quantifying the amount of an activin β_{C} dimer in the biological sample.

In an alternative embodiment, the method may be performed in the reverse way (swapping the capture and tag antibodies). For example, an activin β_{A} antibody may be coated on the plate and an activin β_{C} antibody may be labelled. However, this is less preferable due to the high amounts of activin A (β_{A}-β_{A}) in certain samples which would cause decreased sensitivity of the assay.

In another aspect of the present invention there is provided a method for detecting a propensity for an activin dimer to form in a cell or biological sample, said method comprising detecting a level of activin β_{C} in the cell or biological sample.

Applicants have found that the activin β_{C} subunit can influence activin dimer formation. Its presence will also affect the type of dimer formed. It can dimerise with other activin subunits and hence affect the outcome for homodimers or heterodimers. By competing with other subunits the resultant activin dimers formed will be dependent upon the levels or bioactivity of the β_{C} subunit present. An overabundance of the subunit can preferentially form heterodimers of which one subunit is the activin β_{C} subunit. Similarly, a low level of β_{C} can result in the formation of other dimers of which the β_{C} subunit is not included. Therefore, by considering the level of activin β_{C} in the cell or biological sample, a prediction of the ability or the propensity to form homodimers or heterodimers can be made.

Preferably, the activin dimer that forms is a homodimer or heterodimer, as herein described, depending on the level of the activin β_{C} subunit. Most preferably, the activin dimers are selected from the group including activin AC (β_{A}-β_{C}), activin A (β_{A}-β_{A}), activin BC (β_{B}-β_{C}), activin B (β_{B}-β_{B}), activin CD (β_{C}-β_{D}), activin D (β_{D}-β_{D}), activin C (β_{C}-β_{C}) or activin CE (β_{C}-β_{E}), activin ED (β_{E}-β_{D}), activin E (β_{E}-β_{E}) More preferably the activin dimers are activin AC (β_{A}-β_{C}) or activin A (β_{A}-β_{A}) dimers wherein the β_{C} competes with the β_{A} to make a heterodimer or homodimer.

Activins have diverse roles and various activins and their dimers are involved in growth and differentiation. By predicting the formation of a dimer, then the outcome of a cell or biological tissue can be better predicted. For instance, the formation of activin or inhibin dimers, containing the activin β_{B} subunit, in males may provide predictability of testicular tissue.

The level of activin β_{C} may be measured in by any method which indicates a level of activin β_{C} such as, but not limited to, absolute concentrations from a standard curve, relative to a control sample or immunohistochemically with an antibody reactive to the activin β_{C} subunit. For instance, if a tissue is believed to be potentially cancerous, the level of β_{C} subunit can be measured against normal tissue. Differences in activin β_{C} subunit may indicate to type of subunit formed in the cell or biological tissue. Similarly, just differences in the levels or bioactivity of activin β_{C} in the cell can indicate abnormal tissue.

In another aspect of the invention there is provided a method of diagnosing and/or prognosing a disease or condition associated with activin dimer or dimer formation, the method including detecting an activin β_{C} subunit and/or an activin dimer including an activin β_{C} subunit in a cell or biological sample of a subject. Preferably, the method includes the use of an antibody that recognises an epitope of an activin β_{C} subunit to detect an activin β_{C} subunit and/or an activin dimer including an activin β_{C} subunit in a cell or biological sample of a subject.

In a further aspect of the invention there is provided a method of diagnosing and/or prognosing a disease or condition associated with activin dimer formation, the method including detecting levels or bioactivity of activin β_{C} subunit and/or activin β_{C} dimer formation in a cell or biological sample of a subject. Preferably, the activin β_{C} dimer formation detected is activin AC (β_{A}-β_{C}), activin BC (β_{B}-β_{C}), activin C (β_{C}-β_{C}), activin CD (β_{C}-β_{D}) or activin CE (β_{C}-β_{E}). Most preferably, the activin β_{C} dimer formation detected is activin AC (β_{A}-β_{C}).

An activin dimer may include a homodimer or heterodimer formed by activin subunits selected from the group consisting of β_{A}, β_{B}, β_{C}, β_{D} or PE. Preferably, the activin dimer including an activin β_{C} subunit detected is selected from the group consisting of activin AC (β_{A}-β_{C}), activin BC (β_{B}-β_{C}), activin C (β_{C}-β_{C}), activin CD (β_{C}-β_{D}) or activin CE (β_{C}-β_{E}). Most preferably, the activin β_{C} dimer to be detected is activin AC (β_{A}-β_{C}). An activin dimer present in a cell or biological sample can be detected by general methods of assaying for the specific activin dimer forms. Such assays preferably utilise an antibody that recognises an epitope of an activin β_{C} subunit. Suitable assays for detecting activin dimer formation may preferably include ELISA, immunohistochemistry, immunoprecipitation, immunoaffinity purification or Western Blot techniques.

In a further preferred aspect, the method of diagnosing and/or prognosing a disease or condition associated with activin dimer or dimer formation includes detecting a propensity to form the activin dimers, said method comprising detecting a level of activin β_{C} in the cell or biological sample.

In the methods of the present invention, the disease or condition associated with activin dimers or dimer formation may include diseases or conditions of the liver, prostate, testis, ovary, pancreas, kidney, heart, reproductive organs or skeletal muscle, brain and neural tissue, adrenal gland, pituitary, thyroid gland, stomach, colon, lung, urinary bladder, endometrium, breast, lymph node, skin, salivary gland, bone, nasal cavity, duodenum, gallbladder, uterine cervix, thymus, placenta, fallopian tube, uterus, tonsil, spleen, appendix, seminal vesicle, larynx, tongue, small intestine, rectum, esophagus, myometrium and soft tissue. In particular, the disease or condition may include liver disease (cirrhosis, cancer or hepatitis B and C), lung disease, ovarian cancer, testicular cancer, prostate cancer or prostate enlargement (benign prostatic hyperplasia), pregnancy, endometrial cancer, pre-eclampsia, gestational hypertension and chronic hypertension, inflammatory conditions (eg rheumatoid arthritis, pneumonia, gastrointestinal infection). Preferably the disease is cancer or a tumour. Most preferably the disease is prostate cancer or liver disease.

Applicants have detected activn β_{C} protein in normal and tumours of the following organs: liver, prostate, testis, ovary, pancreas, kidney, brain and neural tissue, adrenal gland, thyroid gland, pituitary, stomach, colon, lung, urinary bladder, endometrium, breast, lymph node, larynx, skin, salivary gland, bone, nasal cavity, duodenum, gallbladder, uterine cervix, thymus, uterus, tongue, small intestine, rectum, esophagus and soft tissue.

Applicants have also detected activn β_{C} protein in the following organs (both normal or disorders of): myometrium, placenta, fallopian tube, tonsil, seminal vesicle, spleen, soft tisssue and appendix.

The present application provides direct evidence of a role for activin β_{C} in prostate disease as exemplified in the Examples. The present application demonstrates the localization of β_{A}, β_{B}, and β_{C} subunits to specific cell types in human liver and benign and malignant prostate. The immunohistochemical localization of activin β_{C} subunits in specific cell types show that activin β_{C} subunit monomer and its homo- or heterodimers may be formed in these cells. In the present invention the method of diagnosing and/or prognosing a disease or condition associated with activin dimer formation may included the use of prostate cells. Prostate cells are taken to include cells derived from the prostate, such as, but not limited to, basal and secretory epithelial and neuroendocrine cells of the prostate and prostatic stroma, smooth muscle, nerve, fibroblast, blood vessel. The prostate cells can be derived from embryonic, foetal or born animals, benign or malignant. The prostate cells may be normal or diseased cells and can include recombinant or mutant cells.

The normal human prostate expresses inhibin and activin subunits. The pluripotent effects of activins and the similarities to transforming growth factor β (TGFβ) suggest a role for activins in progression to malignancy, whereby, the normal growth inhibitory action of activin A observed on benign cells is lost with the acquisition of activin resistance in prostate cancer cells. The mechanisms of rendering tumour cells resistant to activin A may include: dimerisation with activin β_{C} to form novel activin dimers.

Prostate cancer is a leading cause of cancer related death in the male (29). The development of prostate cancer is a multi-step process involving androgens and growth factors, such as members of the fibroblast growth factor (FGF) and transforming growth factor β (TGFβ) superfamilies. Following premalignant changes to the prostate gland, a range of molecular changes occur during the transition to organ-confined and ultimately metastatic disease; growth factors can influence many stages of this progression. Furthermore, the regulatory effects of growth factors are considered to make a significant contribution to the transition from androgen-dependent to androgen-independent disease (30). For example, the role of TGFβ in the progression of prostate carcinoma is well documented (31).

Activin and TGFβ share a number of signalling proteins (e.g. Smads) and the development of resistance to the growth inhibitory effects of TGFβ is a key event in malignant progression (32, 33). Non-malignant prostate has the capacity to express inhibins A and B and activins A, B and AB whereas, malignant prostate tissue can only express the activins. It was shown that activin β_{C} forms dimers with the activin β_{A} and β_{B} subunits in vitro. Thus, if a cell expressed both activin β_{A} (or β_{B}) and β_{C} subunits, a range of new activin dimers could be formed intracellularly. In the prostate gland, activin β_{A} and β_{C} subunit immunoreactivity co-localized to the prostatic basal epithelial cells in the benign prostate and to tumour cells in malignant tissue. So, these cells have the capacity to synthesise new activin β_{C} subunit-containing dimers.

The relative expression of the activin β_{A} and β_{C} subunits could alter the proportions of activin A, activin C or activin AC protein. Theoretically, overexpression of the activin β_{C} subunit leading to an excess of activin β_{C} relative to activin β_{A} subunits would favour the formation of activin C and AC dimers rather than activin A. This would have the effect of reducing the levels or bioactivity of activin A. In the present application activin C had no effect on cell growth in the human prostate tumour cell line, LNCaP, or on the liver tumour cell line, HepG2. No abnormalities were observed in mouse models deficient in activin β_{C} or β_{E} subunits alone or in combination. Therefore the findings support the idea that the activin β_{C} subunit dimers themselves have no ability to regulate prostate tumour cell growth. However, excessive activin β_{C} subunit synthesis can promote activin AC formation and reduce the levels or bioactivity of homodimers of activin A, thereby regulating the levels or bioactivity of bioactive activin A.

The findings support the hypothesis that activins can contribute to malignant progression of prostate cancer. The present application shows that the β_{C} subunit is a candidate for a role in tumour progression.

In yet another aspect of the invention there is provided a method of diagnosing and/or prognosing a disease or condition associated with activin dimer or dimer formation in a subject, the method including the steps of:
(a) contacting a first antibody that recognises an epitope of a first activin β subunit with a biological sample from a subject:
(b) allowing the first antibody to bind to a first activin β subunit in the sample;
(c) washing the sample to substantially remove any unbound material in the sample;
(d) contacting the sample with a second antibody that recognises an epitope of a second activin β subunit, wherein the second antibody is tagged with a labelling agent; and
(e) detecting the labelling agent to identify an activin β_{C} dimer in the biological sample, wherein the first or second antibody recognises an epitope of an activin β_{C} subunit.

Preferably, the activin β_{C} dimer detected is selected from the group consisting of activin AC (β_{A}-β_{C}), activin BC (β_{B}-β_{C}), activin C (β_{C}-β_{C}), activin CD (β_{C}-β_{D}) or activin CE (β_{C}-β_{E}) Most preferably, the activin β_{C} dimer to be detected is activin AC(β_{A}-β_{C}). In the method it is preferred that the first antibody recognises an epitope of an activin β_{C} subunit. Preferably, the second antibody recognises an epitope of an activin β_{A} or β_{B} subunit. More preferably, the second antibody recognises an epitope of an activin β_{A} subunit. Preferably, step (e) includes quantifying the amount of an activin β_{C} dimer in the biological sample. The steps of the method may be performed as previously described for detecting an activin β_{C} dimer.

In the diagnostic and/or prognostic methods of the present invention it is preferred that the subject is a mammalian animal, including but not limited to a human. The biological sample of the subject is preferably a serum sample, such as human serum. The biological sample may be a lysate of human prostate tissue or conditioned media of prostate cells, particularly if the disease or condition to be diagnosed and/or prognosed is prostate cancer. If the disease or condition to be diagnosed and/or prognosed is related to a reproductive disease or condition then the biological sample may include ovarian follicular fluid, seminal fluid or seminal plasma. The biological sample may be derived from a liver sample or fluid produced from the liver, particularly if the disease or condition to be diagnosed and/or prognosed is a liver disease, such as but not limited to, cirrhosis, cancer or hepatitis B or C.

Applicants have detected activin AC protein in samples of human serum from patients with pneumonia, gastrointestinal infection, prostate cancer, ovarian cancer, endometrial cancer, cirrhosis, hepatitis B, hepatitis C and rheumatoid arthritis. Activin AC protein has also been detected in mouse testicular cell line supernatant (i.e. leydig cells, sertoli cells, late spermatocyte and early spermatocyte) and rabbit kidney mesangial cell line supernatant.

In another aspect of the present invention there is provided a composition for detecting an activin β_{C} subunit and/or an activin dimer including an activin β_{C} subunit in a cell or biological sample, wherein the composition includes an antibody that recognises an epitope of an activin β_{C} subunit, and a suitable diluent, excipient or carrier. Preferably, the antibody is a purified antibody that is capable of recognising monomeric or dimeric forms of activin β_{C}. More preferably, the antibody recognises an epitope of activin β_{C} that includes the amino acid sequence VPTARRPLSLLYYDRDSNIVKTDIPDMVVEAC.

Another aspect of the present invention provides a composition for diagnosing and/or prognosing a disease or condition associated with activin dimer formation, wherein the composition includes an antibody that recognises an epitope of an activin β_{C} subunit, and a suitable diluent, excipient or carrier. Preferably, the antibody is a purified antibody is that is capable of recognising monomeric or dimeric forms of activin β_{C}. More preferably, the antibody recognises an epitope of activin β_{C} that includes the amino acid sequence VPTARRPLSLLYYDRDSNIVKT-DIPDMVVEAC.

The compositions as herein before described preferably include a suitable diluent, excipient or carrier that is compatible with the antibody that recognises an epitope of an activin β_{C} subunit. An acceptable carrier, excipient or diluent may include, water, salt solutions, BSA, Triton X-100. Preferably, the compositions are sterile aqueous solutions. The compositions may also contain buffers, diluents and other suitable additives. The compositions may include other adjunct components that are compatible with the antibody that recognises an epitope of an activin β_{C} subunit, such as labelling agents or dyes.

In further aspect of the present invention there is provided a kit for detecting an activin β_{C} dimer in a cell or biological sample, wherein the kit includes a first antibody that recognises an epitope of a first activin β subunit, a second antibody that recognises an epitope of a second activin β subunit, and a labelling agent for tagging the second antibody, wherein the first or second antibody recognises an epitope of an activin β_{C} subunit.

In yet another aspect of the present invention there is provided a kit for diagnosing and/or prognosing a disease or condition associated with activin dimer formation, wherein the kit includes a first antibody that recognises an epitope of a first activin β subunit, a second antibody that recognises an epitope of a second activin β subunit, and a labelling agent for tagging the second antibody, wherein the first or second antibody recognises an epitope of an activin β_{C} subunit.

In the kits of the present invention the first antibody and the second antibody may be antibodies as previously described for the methods of the present invention. The first antibody preferably recognises an epitope of an activin β_{C} subunit. Preferably, the second antibody recognises an epitope of an activin β_{A}, β_{B}, β_{C}, β_{D} or β_{E} subunit. More preferably, the second antibody recognises an epitope of an activin β_{A} subunit. Preferably, the first or second antibody is a purified antibody is that is capable of recognising monomeric or dimeric forms of activin β_{C}. More preferably, the antibody recognises an epitope of activin β_{C} that includes the amino acid sequence VPTARRPLSLLYYDRDSNIVKTDIPDMVVEAC.

A further aspect of the invention is a method of treating or preventing a disease or condition associated with activin dimer formation, the method including controlling levels or bioactivity of activin β_{C} in a subject such that activin dimer formation in the subject is modulated. Preferably the disease or condition is prostate cancer.

Without being limited by theory, a subject may be treated to increase or decrease levels or bioactivity of activin β_{C}. Levels or bioactivity of activin β_{C} may be preferably increased in a cell and/or biological fluid of a subject by introducing regulatory factors that increase the expression of activin β_{C} into a cell, introducing expression vectors that express activin β_{C} into a cell and/or introducing exogenous activin β_{C} into a cell and/or biological fluid of a subject. Levels or bioactivity of activin β_{C} may be controlled using methods as previously discussed.

It is preferred that the method of treating or preventing a disease or condition associated with activin dimer formation includes decreasing levels or bioactivity of active activin β_{C} by the use of an inhibitory molecule. Preferably, the activin β_{C} inhibitory molecule is an antibody against activin β_{C}, an activin β_{C} antisense oligonucleotide or an agent that decreases the expression of activin β_{c} Preferably, the activin β_{C} inhibitory molecule is an antibody (insert). Suitable antisense oligonucleotide sequences (single stranded DNA fragments) of activin β_{C} may also be used to decrease the levels or bioactivity of activin β_{C}. In the method, the activin β_{C} inhibitory molecule can be preferably administered to a subject. More preferably, the inhibitory molecule is administered in a safe and effective amount into a cell and/or biological fluid of a subject.

The method can include administering to a subject in need thereof an effective amount of an agent that decreases the expression of activin β_{C} such that the activin dimer formation is induced. Preferably, the agent is an activin β_{C} inhibitory molecule as discussed earlier.

The term “effective amount” means a dosage sufficient to provide treatment or prevention for the disease or condition being treated or prevented. This will vary depending on the subject and the disease/condition being effected. The effective amounts of an agent used in the methods of the present invention may vary depending upon the manner of administration, the condition of the animal to be treated, and ultimately will be decided by the attending scientist, physician or veterinarian.

The agent, activin β_{C} inhibitory molecule, activin β_{C} regulatory factor and/or activin β_{C} used in the methods as hereinbefore described can be administered systemically or locally to a subject. Systemic administration can be achieved parenterally (e.g. intravenous injection, intramuscular, subcutaneous or intraperitoneal injection, or by implantation of a sustained release formulation), orally, by inhalation, or transdermally (e.g. iontophoretic patch). Local administration to an animal can be achieved by subcutaneous injection, implantation of a sustained release formulation, or transdermal administration. Preferably, the agent, inhibitory molecule, regulatory factors and/or activin β_{C} is administered directly to prostate tissue of a subject. Topical administration in the form of ointments, aqueous compositions including solutions and suspensions, liposomes, micro capsules, creams, lotions, aerosol sprays or dusting powders may be used.

In the present methods of treatment activin β_{C} subunit expression may be increased or decreased by preferably affecting activin β_{C} expression intracellularly, so to either increase or reduce available activin β_{C} subunit for heterodimerisation. Therefore, preferably the agent is inserted into a viral vector, such as gene therapy agent that is prostate and or liver specific.

In another aspect of the present invention there is provided a pharmaceutical composition for treating, preventing or diagnosing and/or prognosing a disease or condition associated with activin dimer formation, the composition including an effective amount of activin or an activin β_{C} inhibitory molecule, and a suitable pharmaceutically acceptable diluent, excipient or carrier. Preferably, the pharmaceutical composition includes an activin β_{C} inhibitory molecule and is suitable for treating prostate cancer.

The activin β_{C} inhibitory molecule in the composition may be any be any molecule capable of blocking the activity and/or expression of activin β_{C}. Activin β_{C} inhibitory molecules may include an antibody against activin β_{C}, an activin β_{C} antisense oligonucleotide or an agent that decreases the expression of activin β_{C}.

Preferably, the activin β_{C} inhibitory molecule suitable for the compositions of the present invention is a purified antibody, wherein the antibody recognizes an epitope of an activin β_{C} subunit. Preferably, the antibody is capable of recognizing monomeric or dimeric forms of activin β_{C}. More preferably, the antibody recognizes a epitope of activin β_{C} that includes the amino acid sequence VPTARRPLSLLYYDRDSNIVKTDIPDMVVEAC. Alternatively, the activin β_{C} inhibitory molecule is an activin β_{C} antisense oligonucleotide or an agent that decreases the expression of activin β_{C}.

The compositions of the present invention can be formulated as pharmaceutical compositions. The compositions may be formulated as solutions, emulsions, or liposome-containing formulations. The compositions may be generated from a variety of components that include liquids, self-emulsifying solids and self-emulsifying semisolids. The pharmaceutical emulsions may also be present as multiple emulsions that are comprised of more than two phases. Pharmaceutical excipients such as emulsifiers, surfactants, stabilisers, dyes, penetration enhancers and anti-oxidants may also be present in the compositions.

Suitable pharmaceutically acceptable carriers can include, water, salt solutions, alcohols, polyethylene glycols, gelatin, lactose, amylose, magnesium sterate, silicic acid and viscous paraffin. Formulations for topical administration may include sterile and non-sterile aqueous solutions. The compositions can also be formulated as suspensions in aqueous, non-aqueous or mixed media. Aqueous suspensions may further contain substances which increase the viscosity of the suspension and may also contain stabilisers. The solutions may also contain buffers, diluents and other suitable additives. The compositions can include other adjunct components that are pharmaceutically compatible with the active components, such as dyes, flavouring/aromatic agents, preservatives, antioxidants, thickening agents.

The compositions can be conveniently presented in unit dosage form and can be prepared according to conventional techniques in the pharmaceutical field. The compositions can be prepared by combining the active compounds/agents with a liquid carrier or finely divided solid carriers or both. The pharmaceutical compositions may be formulated into many forms, such as, tablets, capsules, liquid syrups, soft gels, suppositories or enemas.

The pharmaceutical compositions of the present invention may be formulated and used as foams, including emulsions, microemulsions, creams, jellies and liposomes. The formulations of the above compositions described would be known to those skilled in the pharmaceutical field.

The methods as hereinbefore described may be performed in vitro or in vivo and are applicable to various animal species that express activin β_{C}.

The present invention will now be more fully described with reference to the accompanying Figures and examples that illustrate preferred embodiments of the invention. It should be understood, however, that the description following is a non-limiting example only and should not be taken in any way as a restriction on the generality of the invention hereinbefore described.

### EXAMPLES

### Example 1

### Activin β_{C} Antibody

(a) Antibody Preparation.

A synthetic peptide of sequence (VPTARRPLSLLYYDRDSNIVKTDIPDMVVEAC) corresponding to amino acids 82-113 of the deduced mature human activin β_{C} subunit (6) was synthesized by fluorenylmethoxycarbonyl chemistry. The β_{C} peptide was made corresponding to homologous regions of β_{A} and β_{B} subunits that have been used to generate β_{A} and β_{B} monoclonal antibodies. Outbred female mice of strain TO were immunized with β_{C} peptide). The housing and care of the animals were in accordance with Medical Research Council guidelines. Tail bleeds were obtained at monthly intervals and screened using a standard enzyme-linked immunosorbent assay (ELISA) procedure) for reactivity to β_{C} peptide. After a booster immunization the mice were killed, their spleens were removed, and splenocytes were fused to SP2/0 myeloma cells using a standard fusion protocol with polyethylene glycol (Ninety-six positive clones were chosen and expanded to provide supernatant for further testing by immunohistochemistry.

(b) Recloning, Isotyping, and Purification.

Selected cell populations secreting activin β_{C} antibody were recloned in methylcellulose. The subsequently chosen antibody, clone 1, was isotyped using a Sigma ImmunoType kit (St. Louis, Mo.) and was found to be a mouse IgG1 antibody. Clone 1 antibody was then purified using protein G affinity chromatography (Prosep-G Bioprocessing, Consett, UK).

(c) Specificity Testing of Activin β_{C} Antibody

Cross-reactivity test of the β_{C} antibody with β_{A}, β_{B}, β_{C} and β_{E} peptides. Ninety-six-well plates were coated with synthetic peptides to β_{A}, β_{B}, β_{C}, and β_{E} in bicarbonate buffer. The top row was coated with 1 mg/mL β_{A} peptide, the second row with 1 mg/mL β_{B} peptide, the third row with 1 mg/mL β_{C} peptide, the fourth row with 1 mg/mL β_{E} peptide, and the last row was an uncoated control. After coating, plates were blocked with 100 mL 1% (wt/vol) BSA in phosphate-buffered saline (PBS) containing 1% (vol/vol) H₂O₂ for 30 min. After blocking, purified β_{C} antibody (1.6 mg/mL) was serially diluted from 10²²-10²⁸ and added to the plate. Dilutions were made in Tris conjugate buffer [25 mmol/L Tris-HCl buffer, pH 7.5, containing 0.15 mol/L NaCl, 1% (wt/vol) BSA, and 1% (vol/vol) Tween-20]. After 60 min the plates were washed, and rabbit anti-mouse immunoglobulin/peroxidase conjugate (DAKO Corp., High Wycombe, UK) was added at a dilution of 1:2000 in Tris conjugate buffer and incubated at room temperature for 30 min. After washing, the plate was developed by the addition of 50 mL/well tetramethylbenzidine peroxidase substrate (Dynatech Corp., Billinghurst, UK). The reaction was stopped after 30 min by the addition of 50 mL/well 6% (vol/vol) phosphoric acid. Absorbances were read at 450 nm using a standard microplate reader.

(d) Results—Specificity of β_{C} Antibody

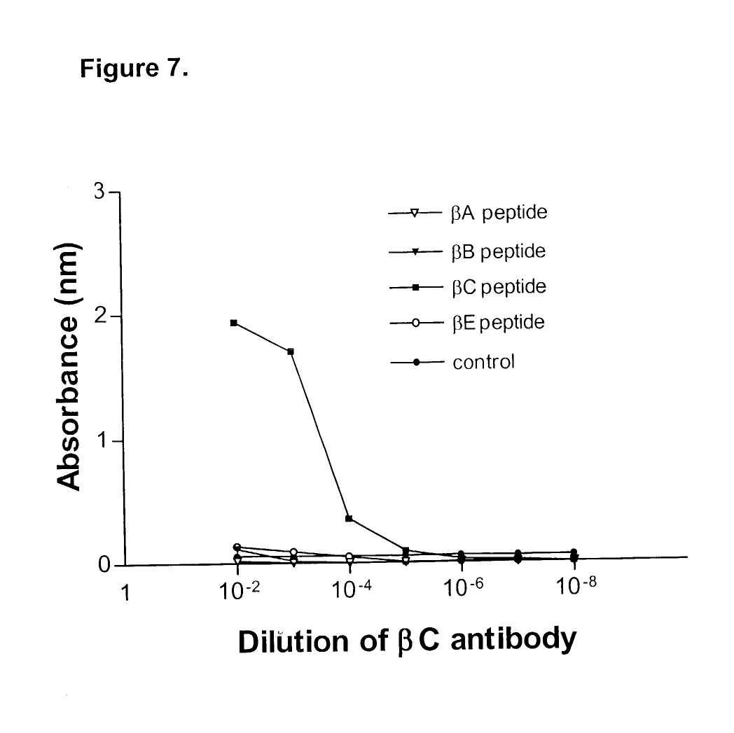

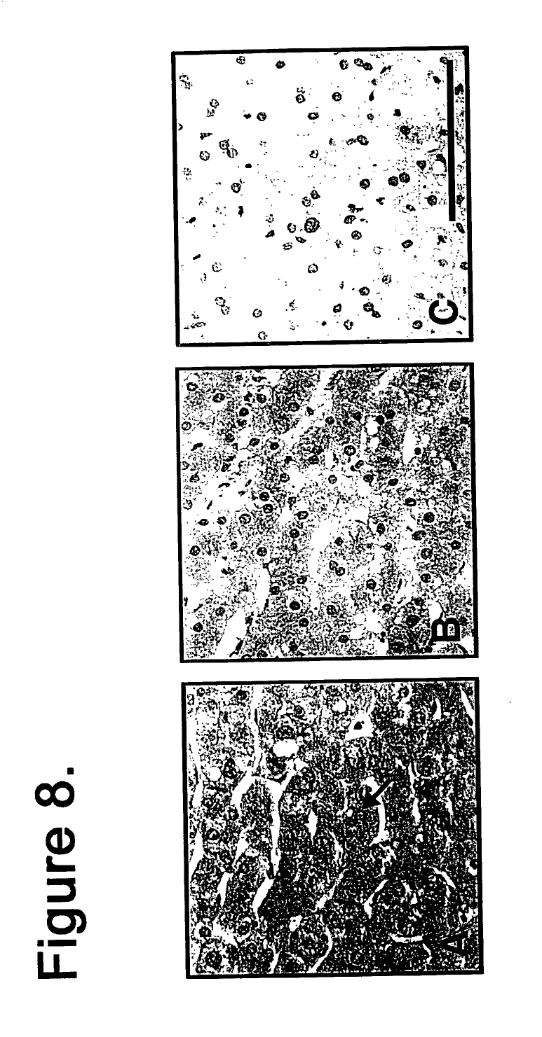

Cross-reactivities of all β_{C} supernatants with β_{A}, β_{B}, β_{C} and β_{E} peptides were tested by ELISA. Clone 1 β_{C} antibody showed minimal cross-reactivity (0.1%) with β_{A}, β_{B} or β_{E} peptides by ELISA, as shown in FIG. 7. Immunohistochemical screening of β_{C} antibody supernatants and purified β_{C} clone 1 antibody was compared on human liver tissue sections. β_{C} subunit immunoreactivity was localized to hepatocytes using the β_{C} clone 1 supernatant (arrow, FIG. 8A) and purified clone 1 antibody (FIG. 8B). Specificity of staining was shown by preabsorption with β_{C} synthetic peptide, which abolished immunostaining (FIG. 8C).

### Example 2

### Immunohistochemistry

(a) Tissues.

Human prostate needle biopsy specimens from 22 men were obtained from Melbourne Pathology (Collingwood, Australia) and consisted of 12 diagnosed with BPH and 10 with high grade prostate cancer (each having a Gleason score of 7-10). Human liver specimens were obtained from the John Radcliffe Hospital (Oxford, UK). Tissue were fixed in buffered formalin and processed to paraffin. Signed consent forms were obtained from patients, and the specimens were used in accordance with the requirements and approval of the standing committees for human and animal ethics and experimentation at Monash Medical Centre and Monash University.

(b) Screening of β_{C} Antibodies on Human Liver.

Tissue culture supernatants were screened by immunohistochemistry on sections of human liver tissue. The protocol followed was identical to that described by Thomas et al. (34) except for the following steps. Sections were dewaxed and placed in 0.01 mol/L glycine buffer solution (pH 4.4). Antigen retrieval involved exposing sections to microwaves at 2.25 watts/mL/min for 3 min followed by 0.3 watts/mL/min for 3 min. All tissue culture supernatants were tested on liver sections and incubated overnight at 4° C. Sections were washed with PBS and incubated for 50 min with biotinylated horse antimouse secondary antibody (DAKO Corp., Botany, Australia) at a dilution of 1:200 in PBS. Sections were washed with PBS and incubated with ABC reagent from the Vectastain Elite ABC Kit (Vector Laboratories, Inc., Peterborough, UK) for 40 min. Peroxidase activity was detected using 3939-diaminobenzidine tetrahydrochloride (Vector Laboratories, Inc.). The reaction was terminated by immersion in distilled water, and the sections were counterstained with Mayers' hematoxylin (Sigma), washed with tap water, dehydrated, and permanently mounted with DPX (BDH, Poole, UK).

(c) Immunohistochemistry using Monoclonal Antibody to the β_{C} Subunit on Human Liver and β_{A}, β_{B}, and β_{C} monoclonal antibodies on human prostate sections. Immunohistochemistry demonstrating β_{A}, β_{B}, and β_{C} subunit localization in BPH patients was performed on serial 3-mm tissue sections. Clone 1 was added to sections of human liver, BPH, and prostate cancer at 5.8 μg/mL and incubated overnight at 4° C. To test the specificity of immunohistochemical staining, the antibody was preabsorbed with the synthetic β_{C} peptide mentioned above at 800 mg/mL for 4 h before addition to sections. β_{A} immunolocalization was reinvestigated using monoclonal E4 antibody, which was used to measure activin A with ELISA. For immunostaining, E4 was used at 2 μg/mL and incubated overnight at 4° C. on tissue sections that had undergone antigen retrieval with 0.01 mol/L Tris buffer (pH 9.7). After antigen retrieval with 0.01 mol/L citrate buffer (pH 6.0), immunolocalization of β_{B} subunit using biotinylated C5 antibody was performed on tissue sections that were swamped with 2 mg/mL E4 for 1 h. Sections were washed with PBS, and β_{B} subunit was detected using 20 μg/mL biotinylated C5 added over-night at 4° C. Sections were then directly detected with ABC (Vector Laboratories, Inc.) for 50 min. Nerve cells were identified in tissue from patients with BPH after antigen retrieval in 0.01 mol/L citrate buffer (pH 6.0), using a monoclonal anti-pan neurofilament antibody (Zymed Laboratories, Inc., San Francisco, Calif.) at a 1:50 dilution added overnight at 4° C. Blood vessels were detected in BPH patients with a monoclonal anti-α-smooth muscle actin IgG (Sigma) added at 6.9 μg/mL for 30 min at room temperature.

(d) Double Immunofluorescence.

Double immunofluorescence was used to investigate whether the stromal staining observed with antibodies to activin β_{B} and β_{C} was localized to smooth muscle cells. After β_{B} and β_{C} subunits had been localized in BPH patients, sections were incubated with double staining enhancer (Zymed Laboratories, Inc.) for 30 min, washed with PBS, and blocked with CAS (CAS-Block, Zymed Labora-tories, Inc.) for 30 min. Monoclonal anti-α-smooth muscle actin IgG (Sigma) was used at 13.8 μg/mL for 2 h. Sections were washed with PBS and incubated with goat antimouse fluorescein isothiocyanate-conjugated antibody (Zymed Laboratories, Inc.) at a concentration of 15 μg/mL for 1 h.

(e) Results: Immunolocalization of the Activin β_{C} Subunit in Human Prostate Tissues

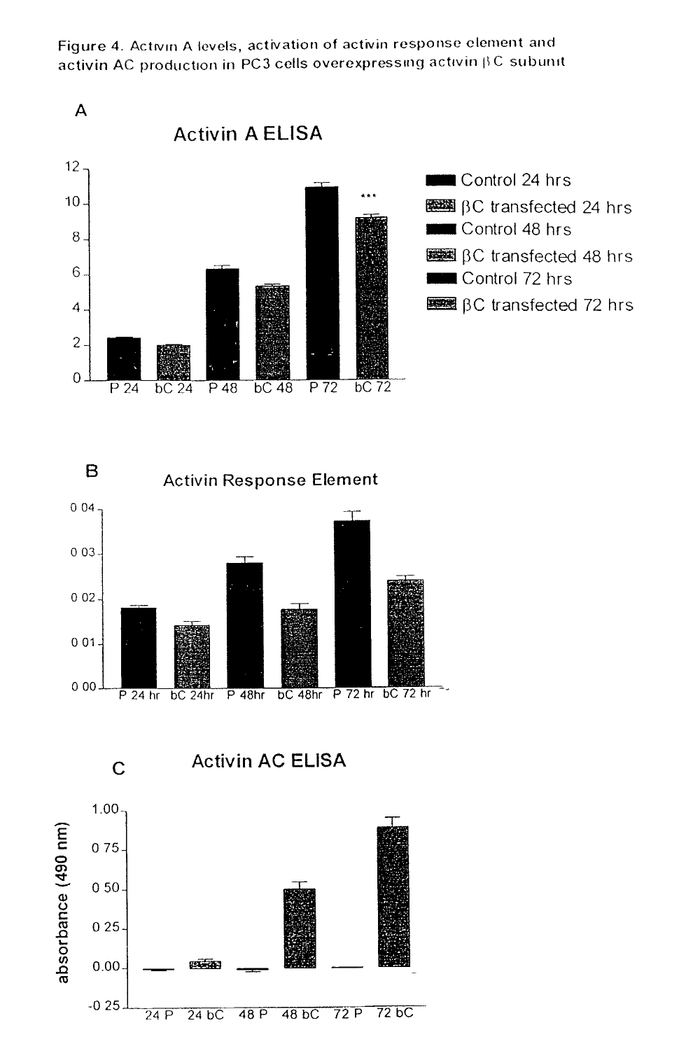

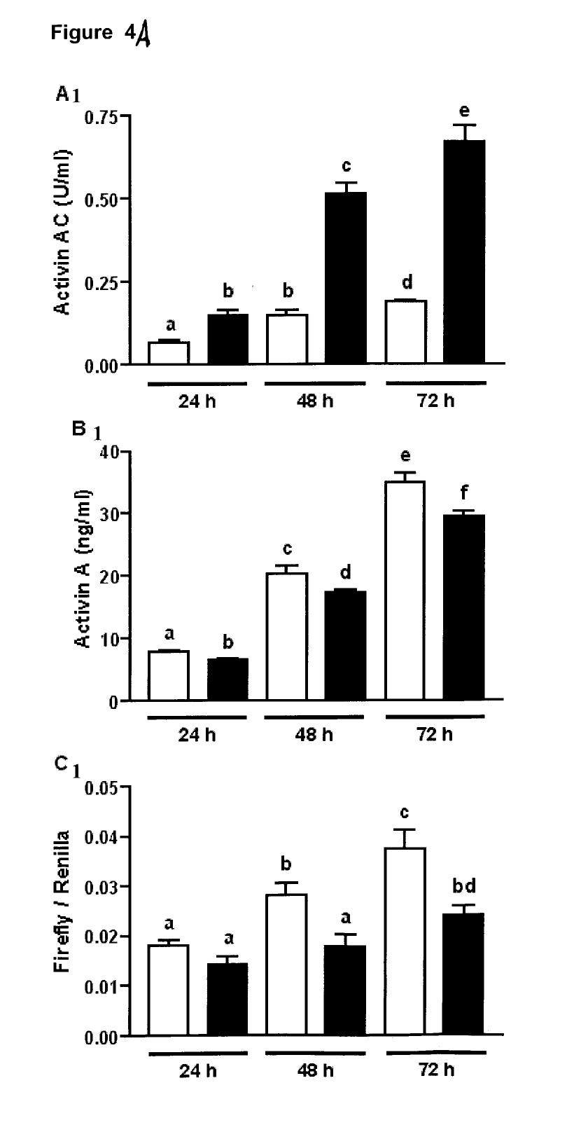

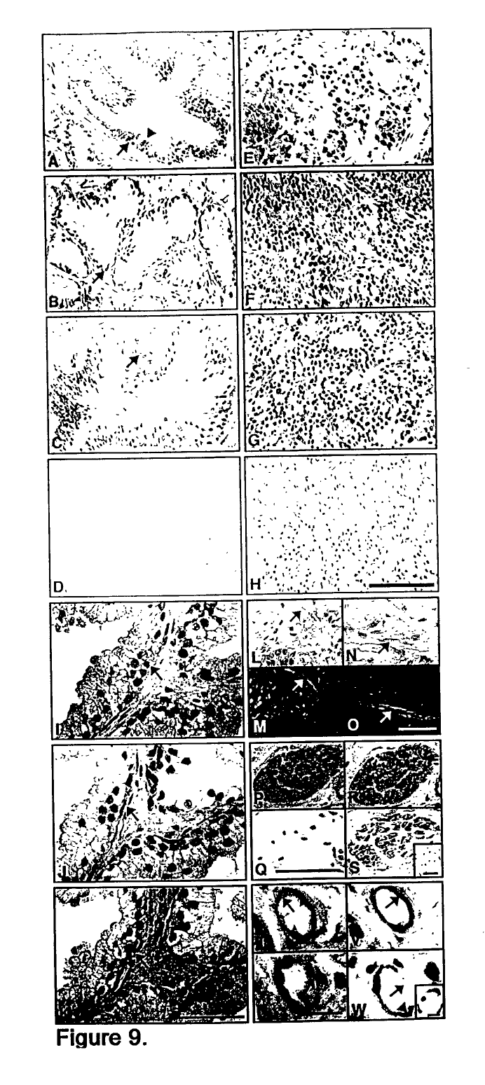

Localization of β_{C} subunit protein relative to that of β_{A} and β_{B} subunits was compared in tissue from patients with BPH (FIG. 9). As previously reported, β_{A} subunit was localized to the basal and secretory epithelial cells (FIG. 9A), whereas β_{B} subunit was localized to the basal epithelial cells only (FIG. 9B). β_{C} subunit immunoreactivity was present in basal epithelial cells (FIG. 9C). No immunoreactivity was detected when the β_{C} antibody was preabsorbed with β_{C} peptide (FIG. 9D). Localization of β_{C} subunit relative to β_{A} and β_{B} subunits in tumour tissue from patients with high grade cancer is shown in FIG. 4. In 10 patients with poorly differentiated prostate cancer, immunoreactivity for β_{A} (FIG. 9E), β_{B} (FIG. 9F), and β_{C} (FIG. 9G) subunits was detected in tumour cells in all patients. Preabsorption of β_{C} antibody with β_{C} peptide abolished staining (FIG. 9H). These patterns of staining suggested that the same cell types contain β_{A}, β_{B} and β_{C}, and to expand this further, serial tissue sections from BPH patients were used. All β_{A}, β_{B} and β_{C} subunits were colocalized to basal epithelial cells (FIG. 91, J, and K, respectively). Because the total thickness of the three serial sections examined was large (0.9 mm) relative to the cell diameter, the boundary pattern of the cells within the focus plane appeared different. In addition, activin β_{B} was localized to stromal cells (FIG. 9L), which were identified as a subset of smooth muscle cells (FIG. 9M). Stromal staining for activin β_{C} (FIG. 9N) was localized to a subset of smooth muscle cells in the stroma (FIG. 90), consistent with β_{B} and β_{C} colocalized in α-actin-positive stroma. In serial tissue sections, β_{A} (FIG. 9P) and β_{C} (FIG. 9R) subunit proteins were localized to nerve cells, which were identified by neurofilament immunoreactivity (FIG. 9S). No immunoreactivity for β_{B} subunit protein (FIG. 9Q) or control mouse IgG (inset) was detected. Using serial sections of prostate tissue, blood vessel smooth muscle was identified by α-smooth muscle actin staining (FIG. 9W). β_{A} (FIG. 9T), β_{B} (FIG. 9U) and β_{C} (FIG. 9V) activin subunits were localized to the cells of blood vessels. No immunoreactivity was detected in the control section (inset).

### Example 3

### Materials for Activin Dimer Analysis

(a) Human Prostate Tumour Cell Lines

The human prostate tumour cell lines, LNCaP and PC3, were obtained from American Type Culture Collection (Rockville, Md.). Cell lines were routinely cultured in DMEM (Life Technologies, Inc., Grand Island, N.Y.) with 10% heat-inactivated FCS(CSL Ltd., Parkville, Australia) and antibiotics (100 lU/mL penicillin and 10 μg/mL streptomycin; CSL Ltd.) in 75 cm² culture flasks (Falcon; Becton Dickinson and Co., Franklin Lakes, N.J.) at 37° C. in a humidified atmosphere of 5% CO₂ in air.

(b) Chinese Hamster Ovary and Pituitary Cell Lines

The CHO cell line was obtained from American Type Culture Collection (Rockville, Md.). The LβT2 cell line was generously given by Pamela Mellon (University of California, San Diego). These cells were originally derived from pituitary tumours induced by the targeted expression of a transgene consisting of 1.8 kb of the rat LHβ promoter linked to the oncogene simian virus 40 T antigen. Both cell lines were maintained in DMEM (Life Technologies) with 10% heat-inactivated FCS(CSL Ltd.)

(c) Growth Factors

Human recombinant activin A and B was purchased from R and D systems (Minneapolis, Minn.). Human recombinant activin C was kindly provided by Biopharm GmBH (Heidelberg, Germany). Activin A and B were stored in 50 μg bovine serum albumin (BSA) per μg activin and were reconstituted with 0.1% BSA in 0.01M PBS. Activin C lyophilised protein was reconstituted in 0.1% trifluroacetic acid/50% acetonitrile, freeze dried, and reconstituted in DMEM+5% FCS for cell culture. Tritiated thymidine ([³H]-thymidine) was obtained from NEN life science Products (Boston, Mass.)

### Example 4

### Homodimer—In Vitro Studies with Activin A and C

(a) [³H]-Thymidine Incorporation/DNA Synthesis

LNCaP and PC3 cells were plated at a density of 5000 and 2500 cells/well, respectively, in DMEM+5% FCS into 96 well plates (Falcon; Becton Dickinson and Co.) for 72 hrs. Media was removed and replaced with activin A, activin B. activin C (40 ng/mL) or vehicle buffer controls and incubated for 2 days. [³H]-thymidine (0.5 uCi/mL) was added to the cells for 20 hrs, after which the cells were harvested using a micromate 196 Cell Harvester (Packard Instrument Co. Meriden, Conn.) and levels of [³H]-thymidine incorporation were determined.

(b) Expression Constructs

Human activin β_{C} cDNA was subcloned into PRK5 expression vector. β_{C} complementary DNA (cDNA) was obtained by RT-PCR using RNA purified from the human prostate tumour cell line DU145. RNA was isolated using the method of Chomczynski and Sacchi (1987) Anal. Biochem. 162:156-159. Total RNA was reverse transcribed to cDNA using oligo(deoxythymidine) and AMV reverse transcriptase (Promega Corp., Madison, Wis.). PCR reactions to amplify the cDNA included the equivalent of 0.3 mg reverse transcribed DU145 RNA, 2.5 U Pfu polymerase (Stratagene, LaJolla, Calif.), 15 pmol of each of the following primer pairs: 1,5′-CCAGCCATGGCCTCCTCATTGCTTCTGGCCTT-3′; and 2,5′-GTAGTCGAAACGACTCTGTCCGGAG-3′ (denaturation temperature of 95° C. for 1 min, annealing temperature of 60° C. for 30 s, extended at 72° C. for 2 min for 35 cycles); 3,5′-GCCCTGTGTCCAGAGCTGCTTTGA-3′ and 4,5′-CGTTTGTGGTCTAAGTGGCTGCTCC-3′ (denaturation temperature of 95° C. for 1 min, annealing temperature of 55° C. for 45 s, extended at 72° C. for 2 min for 40 cycles); and 5,5′-CTGGAGCTGGTACTTGM-GGCCAGG-3′ and 6,5′-GGACACCCACGTCMTCAGATTCGAACC-ATA-3′ (denaturation temperature of 95° C. for 1 min, annealing temperature of 72° C. for 1 min, extended at 72° C. for 2 min for 35 cycles). In separate reactions, 1×Pfu buffer, 2 mmol/L MgCl₂, and 0.2 mmol/L deoxy-NTP (Pharmacia Biotech, Piscataway, N.J.) were used in a final volume of 50 μL. These PCR primers (Integrated DNA Technologies, Coralville, IA) were based on the published sequence for human β_{C} inhibin (6).

The 319-bp product (fragment A) from primer pair 1 and 2 was digested with XbaI/NcoI and gel purified, the 516-bp product (fragment B) from primer pair 3 and 4 was digested with SacI and XbaI, and the 489-bp product (fragment C) from primer pair 5 and 6 was digested with HindIII and SacI. Fragment A (NcoI/XbaI) was ligated to the linkers 5′-AATTCCAGCCAG-3′ and 5′-CATGGTGGCTGG-3′ to generate an EcoRI site at the 5′-end. Full-length C cDNA was obtained by sequential ligation of three cDNA fragments (fragments A, B, and C) into pUC19 (New England Biolabs, Inc., Beverley, Mass. The total fragment was excised with EcoRI and HindIII. A suitable full-length cDNA was subcloned into the pRK5 expression vector as an EcoRI/HindIII fragment. This pRK5 plasmid contains a cytomegalovirus promoter, a polylinker region, a simian virus 40 polyadenylase addition signal, and a simian virus 40 origin of replication.

Cotransfection of cDNAs encoding β_{A} and β_{B} subunits was performed using plasmids. Activin/inhibin cDNAs were transfected, either alone or as cotransfections, into the human embryonic kidney cell line 293. The pRK5 expression plasmid was used as a control plasmid for mock transfections. A total of 5 μg DNA/60 mm dish was used for transfection, and cells were cultured for 48-72 h in serum-free medium and metabolically labeled by culture for 5 h in serum-free, cysteine/methionine-free medium containing 140 mCi [³⁵S]-Translabel (NEN Life Science Products). The supernatant was stored at −20° C.

(c) SDS-PAGE, Immunoprecipitation, and Western Blotting.

Protein samples (10 μL of a total of 500 μL supernatant) were loaded and electrophoresed under reducing or nonreducing conditions using the minigel system (Bio-Rad Laboratories, Inc., Hercules, Calif.) in either 10% or 12% SDS-PAGE.

Gels were fixed in 7% acetic acid/30% methanol for 5 min, treated with Enhance (NEN Life Science Products) for 1 h, and vacuum-dried before exposure to x-ray film at −70° C. for 1-5 days.

pRK5 was kindly supplied by Anthony Mason (Monash University, Melbourne). The reporter construct pGL3.5-5oFSHβ was a gift from William Miller (Department of Biochemistry, North Carolina State University, Raleigh). The reporter construct 3XGRAS-PRL-lux was a gift from Buffy S. Ellsworth (Colorado State University, Ft Collins). The reporter construct p3TP-Lux was a gift from J. Massagué (Memorial Sloan-Kettering Cancer Center, New York). The gsc-lux construct was a gift from J. Wrana (Samuel Lunenfeld Res. Inst., Toronto, Canada). The pSV-β-Galactosidase vector was from Promega Corporation. The PCMVP vector was from Clontech Laboratories (California). The mouse FAST-2 and activin response element (ARE) plasmids were kindly supplied by Yan Chen (Indiana University, Indianopolis). FAST-2 was subcloned into pcDNA3 (Invitrogen) as described in Nagarajan et al, 1999 J Biol Chem, 274:31229-31235 and the pAR3-lux reporter construct consisted of a firefly luciferase gene driven by three tandem repeats of the Mix.2 ARE and a TATA box. The plasmid pRL-CMV, which expresses renilla luciferase under the control of the cytomegalovirus promoter, was from Promega Corporation.

(d) Transient Transfection of LβT2 and CHO Cells

LβT2 cells were maintained in DMEM supplemented with 10% FBS and were cultured at 37° C. in a 5% CO₂ environment. For transient transfection of the pGL3-5.5oFSHβ or 3XGRAS-PRL-lux reporter constructs, 500,000 LβT2 cells/well were cultured in 24 well plates (70-80% confluence) for 24 hours. The Fugene6 reagent (Roche) was then used for transfections at a ratio of 1:3 (μg DNA to μl Fugene6 reagent) according to the manufacturer's instruction. Briefly, cells were transfected with 250 ng reporter construct and 25 ng pCMVβ vector to monitor transfection efficiencies. The activin treatment was applied 24 hours post-transfection; the cells were pre-washed with PBS and the medium was changed to DMEM and 0.2% FBS with the appropriate concentration of activin. The cells were then incubated for a further 24 hours prior to luciferase assays.

CHO cells were maintained in DMEM plus non-essential amino acids (NEAA) and 10% FBS and were cultured at 37° C. in a 5% CO₂ environment. For transient transfection of the p3TP-lux, gsc-lux or the AR3-lux reporter constructs, 50,000 CHO cells/well were cultured in 24 well plates (70-80% confluence) for 24 hours. The Fugene6 reagent (Roche) was then used for transfections at a ratio of 1:3 (μg DNA to μl Fugene6 reagent) according to the manufacturer's instruction. Briefly, cells were transfected with 250 ng reporter construct and 25 ng pSVβ vector to monitor transfection efficiencies. The activin treatment was applied 24 hours post-transfection; the cells were washed twice with PBS and the medium was changed to DMEM plus (NEAA) and 0.2% FBS with or without activin. The cells were then incubated for a further 24 hours.

(e) Luciferase and β-Galactosidase Assay for CHO and LβT2 Cells

Cells were washed twice with ice-cold PBS and then lysed in 200 μl lysis buffer (1% Triton X-100, 25 mM glycylglycine, 15 mM MgSO4, 4 mM EGTA, 1 mM DTT). The cells were then incubated on ice for 30 min before collection of the cell lysate. For the luciferase assay, 50 μl of cell lysate was mixed with 300 μl of Assay buffer (25 mM glycylglycine, 15 mM MgSO₄, 4 mM EGTA, 15 mM potassium phosphate buffer (pH 7.8), 1 mM DTT, 2 mM ATP). The luciferase activity was measured for 2 sec using a Berthold luminometer after injection of the luciferase substrate (luciferin, Promega). For the β-galactosidase assay, 10 μl of supernatant was mixed with 50 μl of Galacton-Star galactosidase substrate (Tropix) and the β-galactosidase activity was counted after a 30 min incubation using a LumiCount 96 well plate reader (Packard). The luciferase activities are represented as relative activities (luciferase activity divided by the β-galactosidaseactivity).

(f) Results: Activin A, B, and C Homodimer Activity

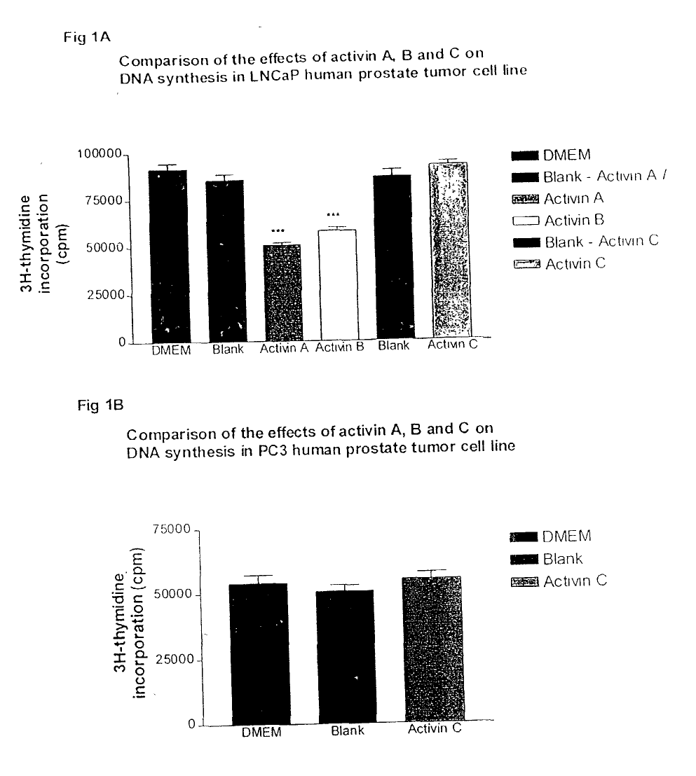

A comparison of the effects of activin A, B and C on DNA synthesis in LNCaP cells is shown in FIG. 1A. Consistent with previous studies, activin A and B significantly inhibited DNA synthesis, at doses of 40 ng/mL, compared to controls. In contrast activin C had no effect. PC3 cells were unresponsive to activin C, as shown in FIG. 1B.

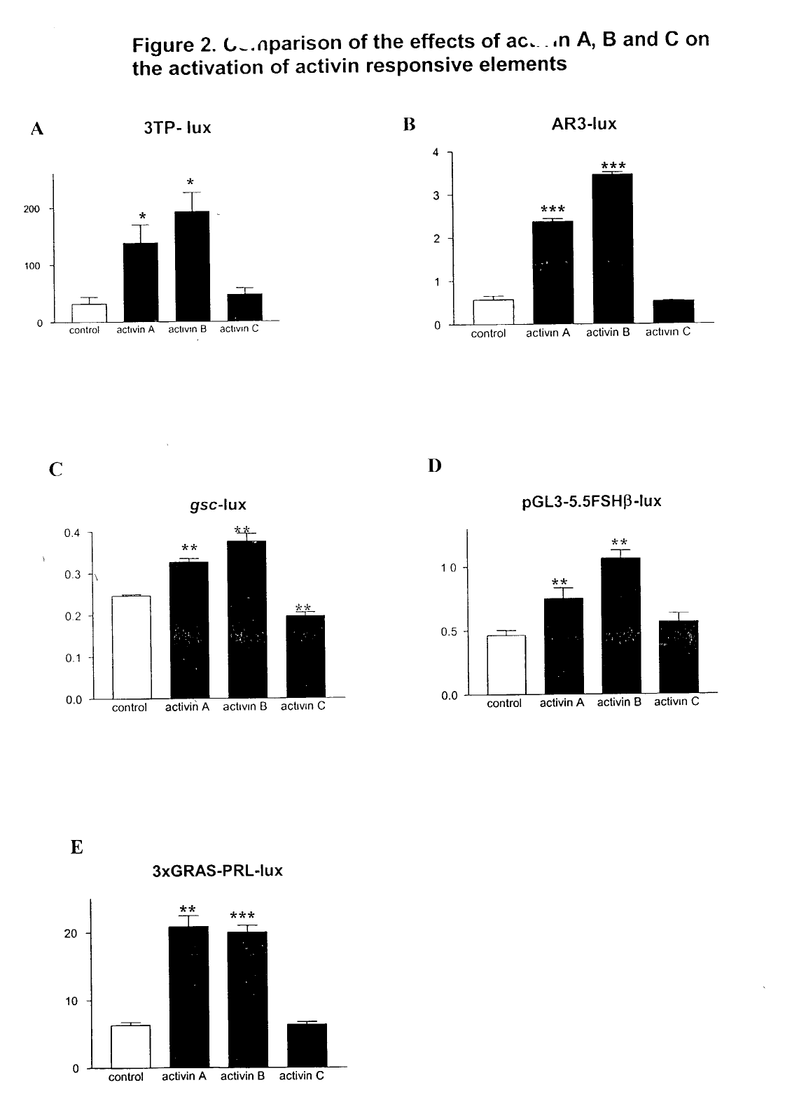

Activin responsive elements were transiently transfected into CHO or LβT2 cells and the effects of exogenous addition of activin A, B and C were determined by relative luciferase expression. In CHO cells, activin A stimulated the TGF-β and activin responsive promoter, 3TP-lux approximately 4.4 fold and activin B approximately 6 fold (FIG. 2A), the activin response element, AR3-lux, was stimulated by activin A, approximately 4 fold and activin B approximately 6 fold (FIG. 2B), and the goosecoid promoter, gsc-lux was stimulated by activin A approximately 1.3 fold and activin B approximately 1.5 fold (FIG. 2C). In the LβT2 cells, activin A activated the FSHβ receptor promoter, pGL3-5.5oFSHβ, approximately 1.6 fold and activin B approximately 2.2 fold (FIG. 2D) and the GnRH receptor activating sequence linked to prolactin, 3XGRAS-PRL-lux was stimulated by activin A approximately 3.3 fold and activin B approximately 3.2 fold ((FIG. 2E). Activin C protein had no effect on any of the activin responsive promoters tested (FIG. 2A-E). Activin C homodimer does not induce activin A or B like responses in these assays.

### Example 5

### Heterodimer In Vitro Studies

(a) Transient Transfection of PC3 Cells

PC3 cells were plated at 200,000 cells/well in DMEM+10% FCS into 12 well plates (70-80% confluence) for 24 hrs. Transient cotransfection combined ARE (1 μg), βC-pRK5 or pRK5 control (2.59 μg) and pRL-CMV (10 ng) control with Superfect (Qiagen), at a ratio of 1:1.7 (μg DNA to μl Superfect reagant) according to manufacturers instructions. Optimisation of this protocol indicated that co-transfection with FAST2 was unnecessary (results not shown). Conditioned media and PC3 cells were collected at 24, 48 and 72 hrs.

(b) Luciferase Assay for PC3 Cells

Cells were washed with PBS and then lysed with 300 μl passive lysis buffer (1×; Promega), while the culture plate was rocked at RT for 30 min. The luciferase assay was performed using the Dual-Luciferase Reporter Assay kit (Promega). Briefly, 20 μl of PC3 cell lysate was added to 96 well luminescent solid assay plates (Costar). 100 μl of Luciferase Assay Reagent (Promega) was added and firefly luciferase measured on a LumniCount 96 well plate reader (Packard). Following the luciferase reading, 100 μl of Stop and Glo reagent (Promega) was added to each well and renilla luciferase was measured as above. Firefly luciferase readings were normalised for renilla luciferase.

(c) Western Blot

SDS-page was performed under reducing conditions using 15% polyacrylamide gel. Media samples, hr-activin A or hr-activin C proteins were diluted 1:2 in reducing buffer (7 mol/L urea, 0.1% NaH₂PO₄.H₂O, 1% SDS and 0.01% bromophenol blue, pH 7.2). Samples were incubated at 100° C. in a heat block for 10 min, centrifuged briefly, gel was run at 200V, constant mAmps for 30 min with running buffer (Tris, glycine, 10% SDS). Immobilon P (PVDF) membrane, which had been pre-incubated in methanol for 15 sec, and milliQ water for 2 min, was equilibriated along with the gel in transfer buffer (0.7 mol/L glycine, 0.3 mol/L Tris and 15.6% ethanol) for 5 min. The proteins in the gel were transferred to the membrane overnight at 30V, 75 mAmps. Following transfer, the membrane was soaked in milliQ water, then methanol for 10 sec, then milliQ water for 2 min. The membrane was blocked (5% Non-fat milk powder, 0.01% Tween in 1×PBS) for 60 min, and washed (1% Non-fat milk powder, 0.01% Tween in 1×PBS) for 3×5 min. Activin β_{C} clone 1 antibody was added at 1:5000 in 1% milk (0.3 μg/mL) in PBS overnight at 4° C. Following washing, the membrane was incubated with goat anti-mouse HRP 1:10,000 in 1% milk in PBS for 2 hours at RT. After subsequent washes, ECL plus substrate (Lumigen Inc., UK) was added according to manufacturer's instructions. The membrane was placed in an x-ray cassette and exposed to X-Omat film (Kodak).

(d) Results: Overexpression of Activin β_{C} cDNA in Human Prostate Tumour Cell Line, PC3

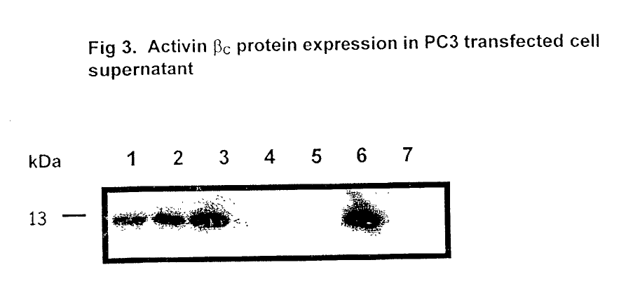

Using a specific antibody to the activin β_{C} subunit, Western blot analysis showed conditioned media from PC3 cells overexpressing the activin β_{C} subunit contained monomeric activin β_{C} subunit protein (FIG. 3). Under reducing conditions a band of 13 kD was detected, similar in size to hr-activin C. No band was detected in PC3 control transfected wells and no cross reaction was detected with hr-activin A.

Endogenous production of activin A in conditioned media of PC3 cells alone or overexpressing activin β_{C} subunit were measured at 24, 48 and 72 hrs of culture. The levels of activin A were significantly lower in PC3 cells overexpressing activin β_{C} subunit compared to controls (FIG. 4A). Associated with a decline in endogenous production of activin A, there was a significant decrease in ARE activation (FIG. 4B).

(e) Results: Evidence of Dimer Formation

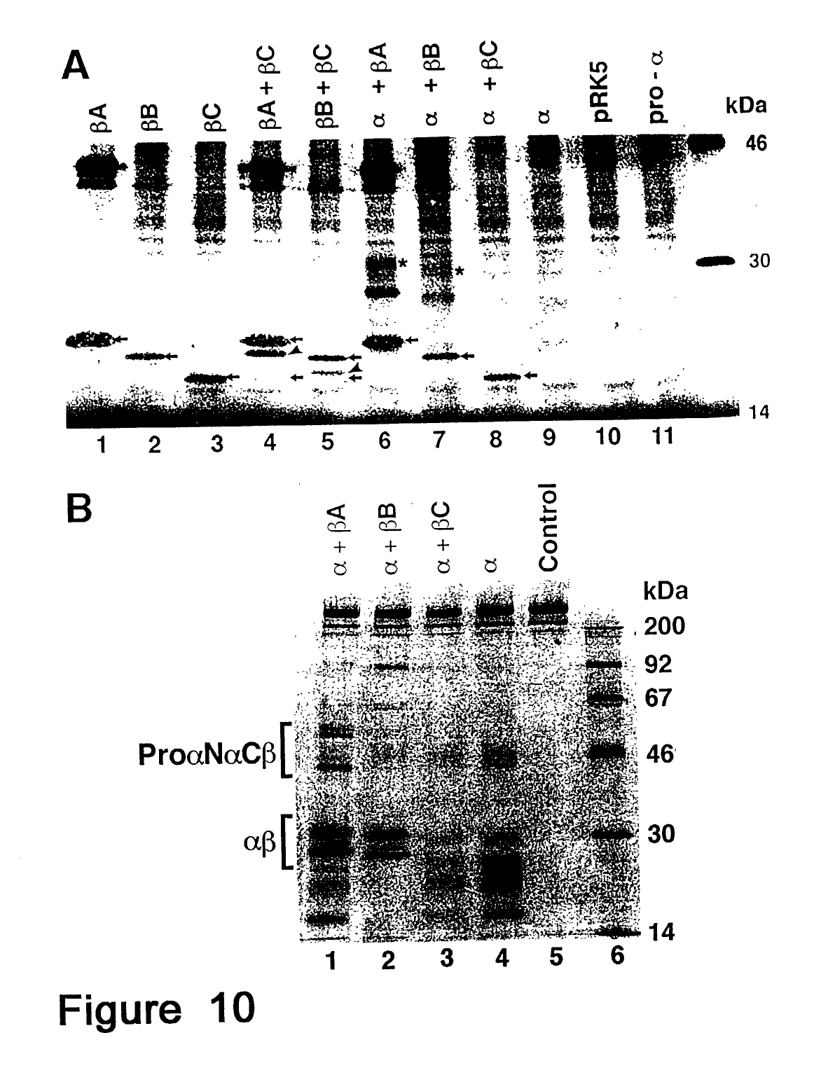

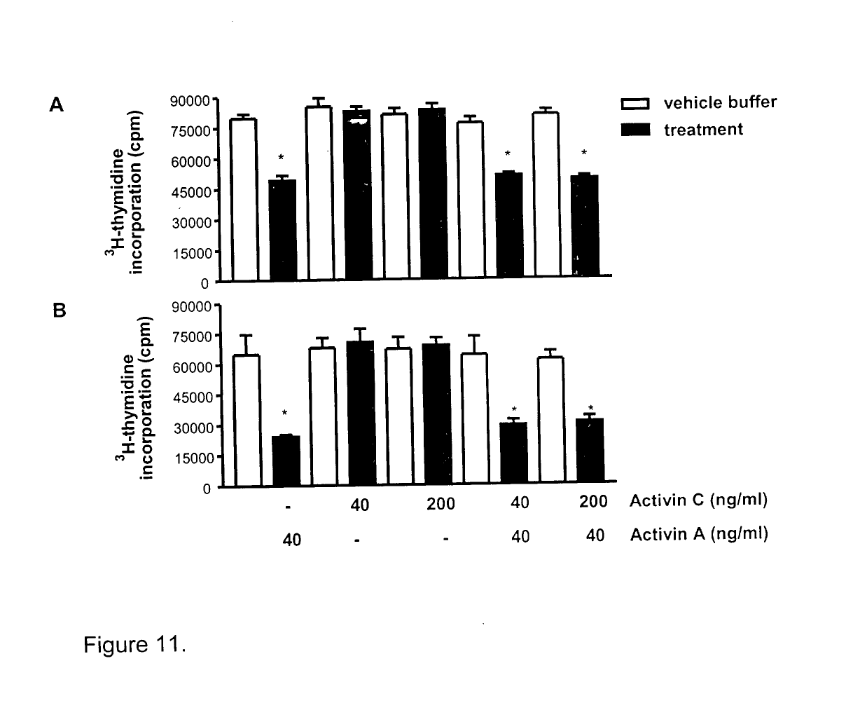

The subunit of β_{C} cloned from DU145 cells was identical to that cloned from human liver with the exception of amino acid 19 (in the signaling sequence), where cytosine was replaced by thymine as designed in the cloning strategy. As shown in FIG. 10A, transfection of β_{A} or β_{B} subunits alone confirmed the formation of homodimers of approximately 24 and 22 kDa dimeric activin A or B, respectively (lanes 1 and 2). Transfection of β_{C} subunits formed β_{C} homodimers, i.e. activin C, with an apparent molecular mass of 20 kDa (lane 3). Cotransfection of β_{A} and β_{C} (lane 4) or β_{B} and β_{C} (lane 5) subunits demonstrates the capacity of β_{C} to heterodimerize with β_{A} or β_{B} and form putative activin β_{C} (23 kDa) or activin β_{C} (21 kDa), respectively. The molecular masses of proteins within complexes was confirmed by running the gel under reducing conditions (data not shown). β_{A} and α subunits dimerize to form mono- and diglycosylated molecular mass forms of inhibin A, and in cells cotransfected with β_{A} and α subunits, inhibin A was detected as well as β_{A} β_{A} and pro-β_{A} proteins (lane 6). Similarly, cotransfection of cells with the β_{B} and α subunit proteins formed mono- and diglycosylated inhibin α and α-β_{B} (lane 7). In contrast, cotransfection of the β_{C} and α subunits did not form heterodimers, and only the β_{C}-β_{C} complex was formed (lane 8). Control lanes consisted of transfection of the α subunit alone (lane 9), transfection of plasmid pRK5 alone (lane 10), and transfection of pro-ainhibin subunit (lane 11). These results suggested that the β_{C} subunit forms homodimers or heterodimers with β_{A} and β_{B} but not inhibin α subunit. The inability of β_{C} to dimerize with the α subunit was confirmed by immunoprecipitation. As shown in FIG. 1B complexes of α-β_{A} (lane 1) and α-β_{B} (lane 2) were immunoprecipitated using α subunit antiserum, but no band corresponding to an α-β_{C} complex was observed (lane 3).

### Example 6

### Activin A ELISA

A specific two site enzyme immunoassay was used to measure total activin A concentrations hr-activin A, was provided by Biotech Australia Pty Ltd (East Roseville, NSW, Australia) was used as a standard. Hr-activin A in 5% BSA.PBS was serially diluted in DMEM+5% FCS to give a range of 2000-7.81 pg/mL. Conditioned media samples from PC3 cells were diluted ⅛ in DMEM+5% FCS. 125 μl media samples, standards or blanks (DMEM+5% FCS) were denatured by the addition of 125 μl 6% SDS in 0.05M PBS and heated to 100 C for 3 mins, then cooled to RT for 20 mins. Oxidation of samples/standards occurred with the addition of 20 μl H₂O₂ for 30 mins at RT. The activin β_{A} antibody E4 coated plates were incubated with 25 μl 20% BSA/assay buffer (0.1M Tris, 5% Triton X-100, 0.9% NaCl, 0.1% azide) prior to the addition of 100 μl duplicates of samples/standards overnight at RT in a moist environment. Plates were washed and 50 μl of biotinylated E4 antibody (Oxford-Bio-innovation) was added at a dilution of 1:80 in 5% BSA/assay for 2 hrs at RT. Following plate washes, 50 μl of strepavidin alkaline phosphatase (Gibco-BRL) was added at a dilution of 1/12 000 in 5% BSA/Tris/Triton assay buffer for 1 hr at RT. Plates were then washed and 50 μl of substrate solution (Gibco-BRL) was added per well and incubated for 1 hr at RT. Subsequently, 50 μl of amplifier solution (Gibco-BRL) was added per well. After colour appeared, the reaction was stopped with 50 μl 0.3M H₂SO₄ and absorbance was read on Multiscan RC microplate reader (Labsystems and Life Sciences International UK Ltd, Basingstoke, UK) using Genesis software (Life Sciences) at 490 nm with a 630 nm reference wavelength.

(a) Activin AC Assay

Activin β_{C} antibody clone 1 as described in Example 1 was coated on a 96 well ELISA plate. Media supernatants and serum samples were added neat, or diluted in DMEM+5% FCS at ½, ¼, ⅛, 1/16 dilutions. Media and serum (125 μl) samples were diluted in 125 μl 6% SDS in 0.05M PBS, and heated to 100° C. for 3 min to denature the samples and cooled to RT for 20 min. The samples were oxidised with the addition of 20 μl H₂O₂ per well for 30 mins at RT. 25 μl 20% BSA assay buffer (0.1M Tris, 5% Triton X-100, 0.9% NaCl, 0.1% azide) was added per well to the β_{C} antibody coated plate, prior to 100 μl duplicates of treated samples being added to each well and incubated overnight in a humidified box. Plates were washed and 50 μl E4-biotinylated antibody (Oxford-Bio-innovation) was added at a dilution of 1/80 in 5% BSA/assay at for 2 hrs at RT. Following plate washes, 50 μl of strepavidin alkaline phosphatase (Gibco-BRL) was added at a dilution of 1/12000 in 5% BSA/Tris/Triton assay buffer for 1 hr at RT. Plates were washed and substrate solution (50 μl/well) was added and incubated for 1 hr at RT. Amplifier solution was added at 50 μl per well and the reaction was stopped with 50 μl 0.3M H₂SO₄. Colour was read at 490 nm with 630 nm reference wavelength on the Multiscan RC microplate reader (Labsystems and Life Sciences International UK Ltd, Basingstoke, UK) using Genesis software (Life Sciences).

(b) Activin AC Enzyme Linked Immunosorbent Assay (ELISA)

Plates were coated and blocked as previously described (35) with human activin β_{C} subunit Clone 1 monoclonal antibody on 96-well ELISA plates (MaxiSorp; Nunc, Roskilde, Denmark). bFF was used as an interim standard. The top dose in the assay, equivalent to a 1/10 dilution, was assigned the arbitrary unitage of 10 U/ml. Standards and samples were diluted in DMEM/5% FCS, as used in the culture experiments. 125 μl of a 6% sodium dodecyl sulphate (SDS) solution in PBS was added (3% final concentration, w/v) to 125 μl of sample or standard, mixed, boiled for 3 minutes and allowed to cool. The addition of PBS to the SDS solution was found to improve the performance of the assay and the linearity of the dose-response curve of the standard and samples. Thereafter, 20 μl of 30% H₂O₂ (2% final concentration, v/v) was added and the tubes incubated at room temperature for 30 mins. To each well, was added 25 μl of 20% BSA/0.1 M Tris/0.9% NaCl/5% Triton X-100/0.1% sodium azide prior to the addition of 100 μl duplicates of the treated samples. Plates were incubated overnight in a sealed humidified box. The next day, the plates were washed with 0.05M Tris/0.9% NaCl/0.05% Tween-20/0.1% NaN₃ before 50 μl biotinylated E4 monoclonal antibody directed to the activin β_{A} subunit (29) in 5% BSA/0.1M Tris/0.9% NaCl/5% Triton X-100/0.1% sodium azide was added to each well and incubated for 2 hours at room temperature. After washing, alkaline phosphatase linked to streptavidin (Invitrogen Corporation, Carlsbad, Calif.) was added to the wells and incubated at room temperature for one hour. After further washes, the alkaline phosphatase activity was detected using an amplification kit (ELISA Amplification System; Invitrogen) whereby the substrate was incubated for one hour at room temperature, followed by the addition of an amplifying reagent. The reaction was stopped with the addition of 50 μl of 0.4M H₂SO₄. The plates were read at 492 nm with a 630 nm reference filter on a Multiskan RC plate reader (Labsystems, Helsinki, Finland) and data were processed using Genesis Lite EIA software (Labsystems). The assay was optimised and assessed for performance, specificity, accuracy and precision.

(c) Detection of Relative Levels or Bioactivity of Activin AC in PC3 Transfected Cells

A specific ELISA as described in Example 6(b) was used to measure the levels of Activin AC (FIG. 4C). Using this activin AC ELISA, PC3 cells expressing activin β_{C} subunit produced increasing levels of activin AC protein between 24 and 72 hours of culture, whereas no activin AC was detected in control cells.

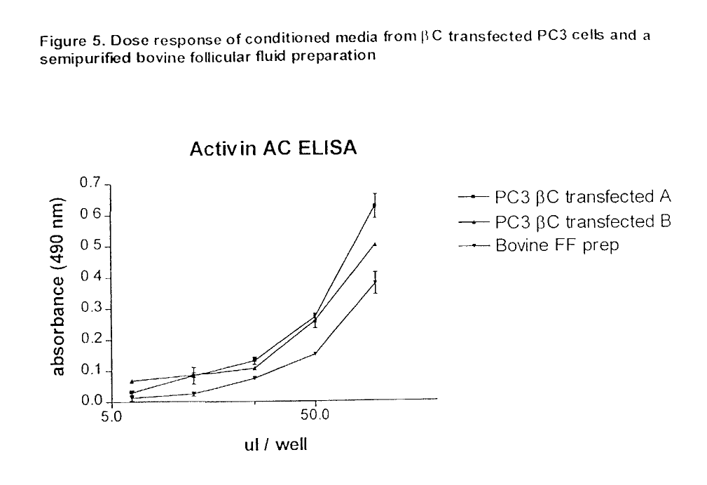

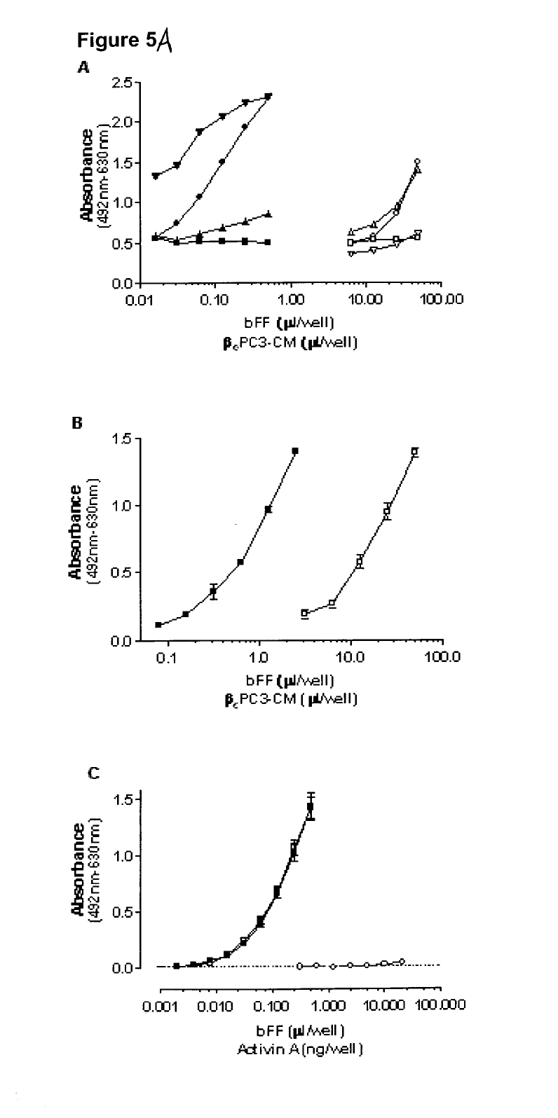

Activin AC was detectable in a semipurified bovine follicular fluid preparation (bFF prep), which was subsequently was used as an interim standard. Dose response curves of the bFF prep and conditioned media from activin β_{C} transfected PC3 cells are shown in FIG. 5. The bFF and media samples, diluted in unconditioned media, diluted out in a linear fashion. Linear regression analysis of log-log transformed data showed that the 95% confidence limits of the slopes overlapped, indicating that the slopes of bFF prep and the media samples were parallel. Activin AC was undetectable in an inhibin standard (results not shown).

(d) Activin AC Heterodimer Protein Formation, In Vitro

In order to investigate the functional consequences of activin β_{C} subunit overexpression in prostate tumor cells, the PC3 cell line was transfected with an expression vector consisting of the human activin β_{C} subunit cDNA driven by the CMV promoter. The PC3 cell line was chosen for this series of experiments because it produces measurable amounts of activin A homodimer (36), therefore, formation of the putative activin AC heterodimer could potentially be observed in this cell line upon overexpression of the activin β_{C} subunit.

Supernatant and cells from the same PC3 cell transfection experiments were assayed with both the activin AC and activin A ELISAs and for ARE promoter activation in parallel. The aim of these experiments was to determine whether the overexpression of the activin β_{C} subunit in PC3 cells resulted in the production of activin AC heterodimer, a concomitant variation in the production of activin A homodimer causing a change in activation of the ARE.

Endogenous activin AC was detected at low levels in cells transfected with the control vector, pRK5 alone (FIG. 4A A1, open bars) and a small but significant increase was observed from 24 to 48 and 72 hours of culture post-transfection (p<0.05). Overexpression of the βC-subunit in PC3 cells resulted in increased levels of secreted activin AC protein (FIG. 4A A1, closed bars) compared to cells transfected with the control vector (open bars). Production of activin AC also increased significantly over a 72 hour time period (p<0.001) in conditioned media from PC3 cells overexpressing the activin β_{C} subunit (FIG. 4A A1, closed bars). Notably, at each individual time point, activin AC production was significantly higher in the supernatant from activin β_{C} subunit overexpressing cells PC3 cells, than in the conditioned media from control vector transfected cells (p<0.01).

In order to determine whether the overexpression of the β_{C}-subunit affects the production of endogenous activin A dimer, activin A homodimeric protein was measured using the same conditioned media samples (FIG. 4A B1) as described above. Activin A production increased significantly from 24 to 48 and 72 hrs of culture in both the control PC3 cell supernatant (p<0.001; FIG. 4A B1, open bars) and PC3 cells overexpressing the activin β_{C} subunit (p<0.001; FIG. 4A B1, closed bars). However, endogenously produced activin A was significantly lower at each individual time point in conditioned media from activin β_{C} subunit overexpressing PC3 cells, when compared to corresponding control samples (p<0.001).

The decrease of activin A production associated with overproduction of activin AC suggests that the cells overexpressing the β_{C} subunit may exhibit lower activin activity. To test this hypothesis, PC3 cells were co-transfected with the activin-responsive reporter construct, pAR3-lux, with or without the activin β_{C} subunit expression vector (FIG. 4A C1). Increasing levels of pAR3-lux activity were observed from in a time-dependent manner in PC3 cells transfected with the control vector (FIG. 4A C1, open bars). with the first statistically significant increase recorded after 48 hour post-transfection. In contrast, activation of pAR3-lux in activin β_{C} subunit overexpressing PC3 cells (FIG. 4A C1, closed bars) was delayed, with a statistically significant increase only after 72 hours post-transfection (p<0.01). Relative luciferase activity in PC3 cells overexpressing the activin β_{C} subunit was significantly lower at 48 hrs (p<0.001) and 72 hrs (p<0.001) when compared with PC3 cells transfected with the control vector alone, but not at 24 hrs. Therefore, in the PC3 cells an increase in endogenously produced activin AC heterodimer was associated with a significant decline in endogenous production of activin A homodimer, in addition to a significant decrease in pAR3-lux activation at 48 and 72 hrs. Significantly lower pAR3-lux activation was not observed at 24 hrs. Whilst activin A protein levels were significantly decreased at 24 hrs this change was relatively small.

(e) Development of an ELISA to Measure the Activin AC Heterodimer

(i) Standard

No purified or hr-activin AC heterodimeric protein is currently available. bFF which has been shown to have high levels of activin A and AB was found to give a strong signal in the activin AC ELISA, serial dilutions showed a linear dose-response curve so bFF was subsequently used as an interim standard. The range of the standard curve was 0.002 μl bFF/well to 10.5 μl/well which was assigned an arbitrary unitage of 0.04 U/ml to 10 U/ml.

(ii) Sample Treatment

It has been shown previously that pre-assay sample denaturation and oxidation resulted in an increased response in similar immunoassays using the E4 monoclonal antibody directed to the activin β_{A} subunit (Knight et al, 1996). The bFF standard serially diluted in unconditioned culture media and an activin β_{C} subunit-transfected PC3 conditioned media sample were assayed with and without pre-treatment to assess the performance in this assay (FIG. 5A A).

With no sample pre-treatment, the bFF and the media sample gave little to no response with relatively high blank values (FIG. 5A A, open and closed squares, respectively). The addition of the denaturation step with SDS/PBS and boiling improved the signal to a small extent (FIG. 5A A, open and closed triangles). An oxidation step improved the signal markedly, with reduced blanks but whilst the standard curve was shifted to the left, it did not demonstrate a linear dose-response curve and was not parallel to the sample (FIG. 5A A, open and closed inverted triangles). Combining both the denaturation step and the oxidation step resulted in a decreased blank, a good response in the assay and a linear dose-response (FIG. 5A A, open and closed circles). When this method was employed, both the bFF standard and the activin β_{C} subunit-transfected PC3 conditioned media sample gave linear dose-response curves that were parallel to each other as shown by a comparison of slopes with overlapping 95% confidence limits of log transformed data (FIG. 5A B, closed and open circles, respectively).

(iii) Accuracy and Precision

The accuracy of the assay was determined by spiking media samples with a known amount of bFF, equivalent to 1.0 U/ml, to determine the percentage recovery of activin AC. Mean recoveries were 97.6±7.3%, from 4 samples on each of 8 plates indicating that quantitative recoveries were achieved from the test samples. The mean intra-plate % coefficient of variation (% CV) was 6.5% and the inter-plate % CV was 3.9%. The limit of detection, defined as the standard dose equivalent to the mean+2 standard deviations of the absorbance of the blank replicates (n=6), was 0.04 U/ml.

(iv) Specificity

Cross-reactivity in the assay of related proteins was impossible to quantify without a purified source of activin AC of known mass. However, no interference or cross-reactivity was detected in the assay when high concentrations of activin B or C in the dose range of 15.63 ng/ml to 500 ng/ml were added (data not shown). In addition, mean recoveries of a known amount of the bFF in the presence of either activin B or activin C, (15.63 ng/ml to 500 ng/ml) were 96.0±3.1 and 100.9±5.7 (n=6) respectively. Since both the bFF used as the standard and the conditioned media samples contain large amounts of activin A, the cross-reaction of activin A was assessed in several ways to determine the ability of the ELISA to accurately measure activin AC dimer. The bFF standard used in the activin AC ELISA contains the equivalent of 0.91 to 234 ng/ml (0.045-11.7 ng/well) activin A as determined using the activin A ELISA. When a range of doses of activin A (0.313-20 ng/well, 6.25-400 ng/ml) was added alone in the activin AC ELISA, a small effect was seen only above a dose of 20 ng/well which is equivalent to 100 ng/ml (FIG. 5A C). Additionally, a dose of 50 ng/ml (2.5 ng/well) activin A was added to each of the doses of bFF in the standard curve. This is greater than the activin A concentration in the β_{C} transfected PC3 conditioned media samples (<40 ng/ml, FIG. 4A C1). There was no displacement of the curve, nor was there any significant difference (p=0.975) when compared to the curve of the normal “unspiked” standard curve (FIG. 5A C). This indicates that activin A does not have a significant effect or cross-reaction in the activin AC ELISA and that the activin AC heterodimer concentrations measured are not affected by the presence of activin A homodimer in the samples. To assess the potential interference in the assay from follistatin, 1 U/ml bFF was pre-incubated with a range of doses of either hr-FS288 and bovine FS. FS concentrations up to 1 μg/ml had no effect on the assay, demonstrating that the assay can measure total activin AC even in the presence of follistatin, bound or unbound (data not shown).

(f) Activin AC Heterodimer Formation, In Vivo

Activin AC protein levels (U/ml) were measured in samples of human serum, normal and malignant human cell line supernatants, human tissue homogenate samples and biological fluids (see Table 1 below).

Changes in activin AC levels (compared to control serum) were observed in serum from patients with pneumonia, gastrointestinal infection, end stage cirrhosis and liver failure, prostate cancer, hepatitis B, and advanced hepatitis C.

Activin AC protein could be measured in human cell lines including; ovarian cancer cell line, primary endometrial cell line, endometrial adenocarcinoma cell line and rheumatoid arthritis cell line.

Activin AC protein could be measured in animal cell lines including; murine late spermatogonial/early spermatocyte cell line, murine leydig cell line, murine spermatogonial cell line and rabbit kidney mesangial cell line. Activin AC protein was detected in human follicular fluid, prostate homogenate from a patient with benign prostatic hyperplasia and serum from a sheep with acute inflammation.

TABLE 1

| Sample | Activin AC (U/ml) |
|---|---|
| Normal serum | |
| Male serum control | 0.033 |
| Female serum control | 0.038 |
| Inflammation | |
| Pneumonia serum | 0.094 |
| Gastrointestinal Infection serum | 0.090 |
| Sheep acute Inflammation model | 0.080 |
| Liver | |
| Hepatitis B serum | 0.074 |
| Advanced hepatitis C serum | 0.098 |
| Cirrhosis & liver failure serum | 0.066 |
| Prostate | |
| Prostate Cancer serum | 0.166 |
| Benign Prostatic Hyperplasia tissue | 0.231 |
| homogenate | |
| Ovary | |
| Human follicular fluid | 0.080 |
| Human ovarian cancer cell line | 0.262 |
| Control media | 0.176 |
| Endometrium | |
| Endometrial adenocarcinoma cell line | 0.197 |
| Control media | 0.174 |
| Normal endometrial cell line | 0.265 |
| Control media | 0.159 |
| Arthritis | |
| Rheumatoid arthritis cell line | 0.183 |
| Control media | 0.168 |
| Testicular cells | |
| Mouse late spermatogonial/early | 0.084 |
| spermatocyte cell line supernant | |
| Mouse spermatogonial cell line | 0.087 |
| supernatant | |
| Mouse leydig cell line supernatant | 0.023 |
| Mouse sertoli cell line supernatant | 0.035 |
| Kidney | |
| Rabbit mesangial cells from | 0.153 |
| glomerulus | |

### Example 7

### Rat Activin β_{C} Immunohistochemistry

(a) Animals

Intact male Sprague-Dawley outbred rats from days 0 to 15 were killed. Ventral prostate lobes were micro-dissected from newborn rats and processed for immunohistochemistry. All animals were obtained from Central Animal Services, Monash University. Two sets of six ventral prostate lobes at each age (days 0, 2, 4, 8 and 15) were used for immunohistochemistry. Tissues were fixed in paraformaldehyde, processed to paraffin, and 3 mm serial sections were cut.

(b) Immunohistochemistry

Immunohistochemistry was performed as described in Example 2 with the following modifications. Sections for β_{C} activin immunostaining with activin β_{C} monoclonal antibody were subjected to microwave antigen retrieval in 0.1 M glycine buffer (pH 4.4). Sections for high molecular weight cytokeratins (HMW) and smooth muscle α-actin immunostaining were subjected to microwave antigen retrieval in 0.01 M citrate buffer (pH 6.0) and then incubated with 0.01% trypsin, 0.2% CaCl₂ for 10 min. All sections were then treated with 3% (vol/vol) H₂O₂ in methanol for 30 min, and blocked with CAS block (Zymed Laboratories, San Francisco, Calif.). Sections were then incubated with primary antibodies or controls overnight at 4° C. (for activin β_{C} antibody) or 2 h at room temperature (HMW cytokeratin). Sections were incubated with biotinylated goat anti-mouse IgG (Zymed) at 1:200 for 60 min at room temperature and then incubated with Vectastain Elite ABC kit (Vector) for 45 min and colour-reacted with 3,39-diaminobenzidine tetrahydrochloride (DAB). All reactions were stopped in water, and sections were counterstained with Mayer's haematoxylin, dehydrated, cleared, and mounted.

For double-labeling of high molecular weight cytokeratins and smooth muscle α-actin, sections were stained for high molecular weight cytokeratins as described above. Before counterstaining, sections were incubated with double stain enhancer (Zymed) for 10 min. After rinsing with PBS, sections were incubated with CAS (Zymed) followed by anti-smooth muscle α-actin for 1 h and detected with peroxidase-labeled polymer (Dako Envision System). Sections were colour-reacted with Vector peroxidase substrate kit (Vector VIP; Vector), counterstained with Mayer's haematoxylin, dehydrated, cleared, and mounted.

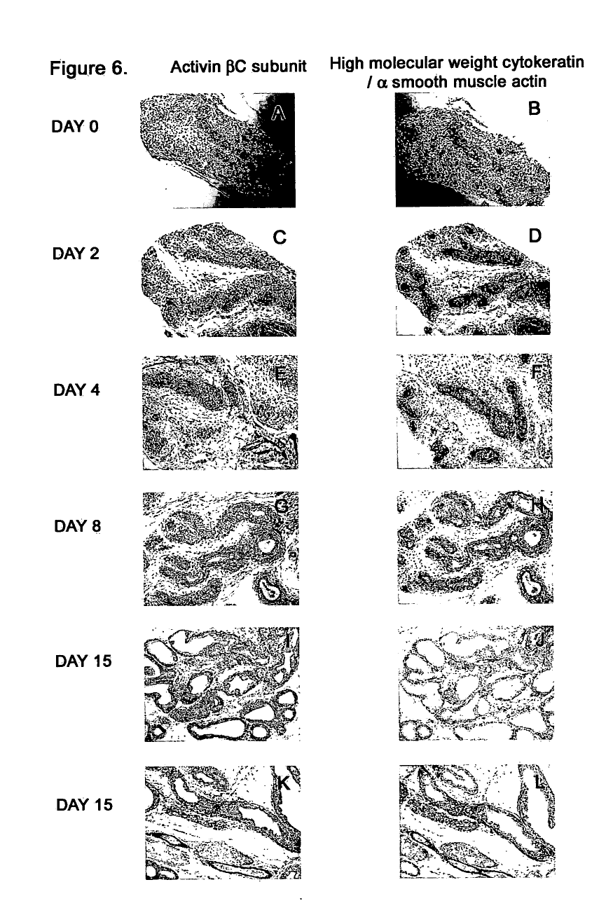

FIG. 6 shows localization of activin β_{C} subunit, high molecular weight (HMW) cytokeratins and α smooth muscle actin in the developing rat ventral prostate lobes at day 0 (A, B), 2 (C,D), 4 (E, F), 8 (G,H) and 15 (I, J, K, L). Activin α_{C} subunit immunolocalization (brown staining) shown in A, C, E, G, I and K and high molecular weight (HMW) cytokeratins (brown staining) and α smooth muscle actin (purple staining) shown in B, D, F, H, J and L. Immunoreactivity for activin β_{C} subunit was localized to the solid epithelial buds on days 0-4 (A, C, E) which were positive for HMW cytokeratin (B, D, F). At day 8, activin β_{C} subunit immunoreactivity was also observed in the epithelial cells of more mature canalising ducts (G). Activin β_{C} subunit protein was also immunolocalized to smooth muscle cells from day 2-8, which was identified by α smooth muscle actin immunoreactivity (B, D, F, H). At day 15 strong activin β_{C} immunoreactivity was observed in columnar epithelial cells (I, K) and smooth muscle sheaths (K), as identified with α smooth muscle actin (L). Activin β_{C} immunoreactivity was also observed in fibroblastic stroma from day 4-15.

### Example 8

### Activin β_{C} Subunit Protein Immunohistochemistry in Normal/Diseased Human Tissues and Animal Tissues

(a) Human Tissues

Human normal tissue array (AA) and human tumor tissue array (BB) were obtained from SuperBioChips Laboratories (Seoul, Korea).

(b) Animal Tissues

Ovaries were removed from adult female cows following abattoir culling. Bovine ovary tissue was fixed in 4% paraformaldehyde, processed to paraffin and 3 μm tissue sections were cut.

The left sagittal brain was removed from a transgenic mouse with a neurodegenerative disorder (familial amyotrophic lateral sclerosis) and corresponding wild type animals (38). The tissue was fixed in 4% paraformaldehyde, processed to paraffin and 3 μm tissue sections were cut.

Ewes were killed by i.v. injection of 20 ml of Lethobarb (Virbac, Peakhurst, NSW, Australia). The heads were then perfused with 21 ml of heparinized saline followed by 11 ml of 10% formalin fixative solution and 0.51 ml of the same fixative solution containing 20% sucrose. The brain blocks were left overnight in the same fixative containing 30% of sucrose and then in 30% sucrose in PBS until they sank. The brain blocks were then frozen in dry ice, wrapped parafilm and stored at −20° C. until sectioning. Coronal sections (7 μm) of sheep pituitary were cut on a cryostat, thaw mounted onto superfrost slides and stored at −200 until used. Coronal sections of sheep brain (40 μm) were cut on a cryostat, collected into individual tissue culture wells containing cryoprotectant and stored at −20° C. until used.

After being de-paraffinated the tissue underwent a pretreatment step of microwave heating in 0.1M glycine (pH 4.5). The sections were immunostained for activin β_{C} subunit protein using the DAKO Autostainer (DAKO, Carpinteria, USA). Briefly, endogenous peroxidase was blocked by incubation of sections with 0.03% H₂O₂ for 5 minutes (DAKO, Carpinteria, USA). After incubation with CAS Blocking solution (Zymed, CA, USA) for 10 minutes, the sections were incubated with activin β_{C} antibody (working concentration 0.45 μg/ml) for 60 minutes. The antibody was detected by incubation with Envision polymer-anti-mouse-horse radish peroxidase (DAKO, Carpinteria, USA) for 15 minutes and visualised by reaction with diaminobenzidine (DAB) (DAKO, Carpinteria, USA) for 5 minutes. The specificity of immunostaining was examined by pre-incubation of primary antibody with 100-fold (w/w) excess of corresponding activin β_{C} subunit peptide.

(c) Immunolocalisation of Activin β_{C} Subunit Protein in Normal and Diseased Human and Animal Tissues.

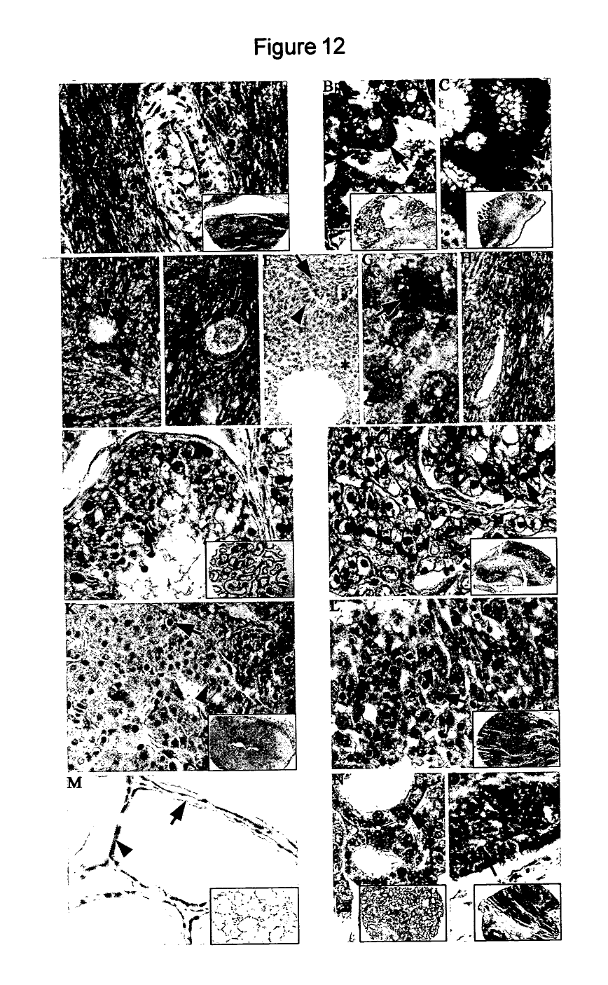

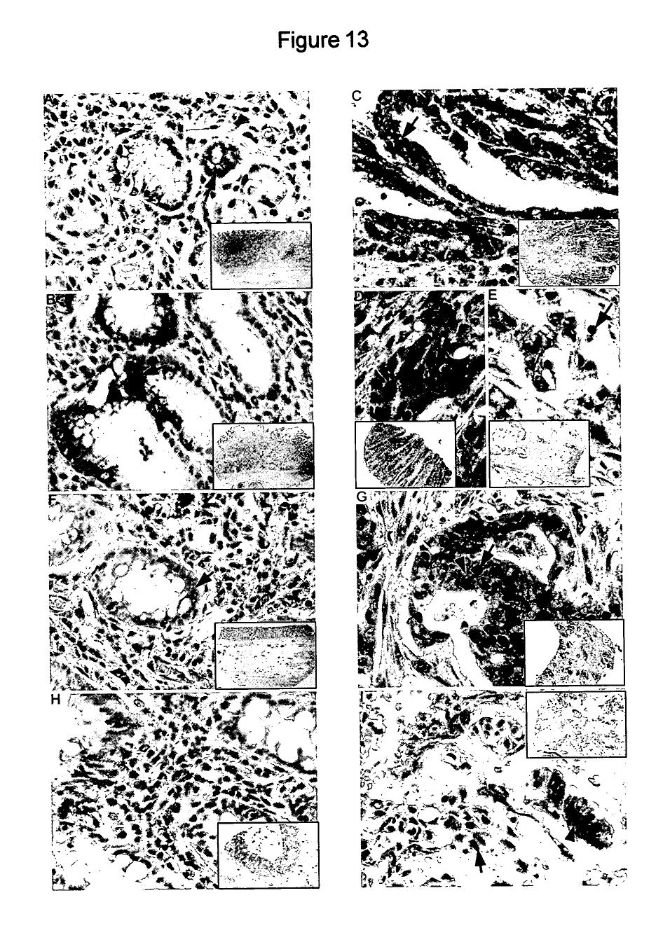

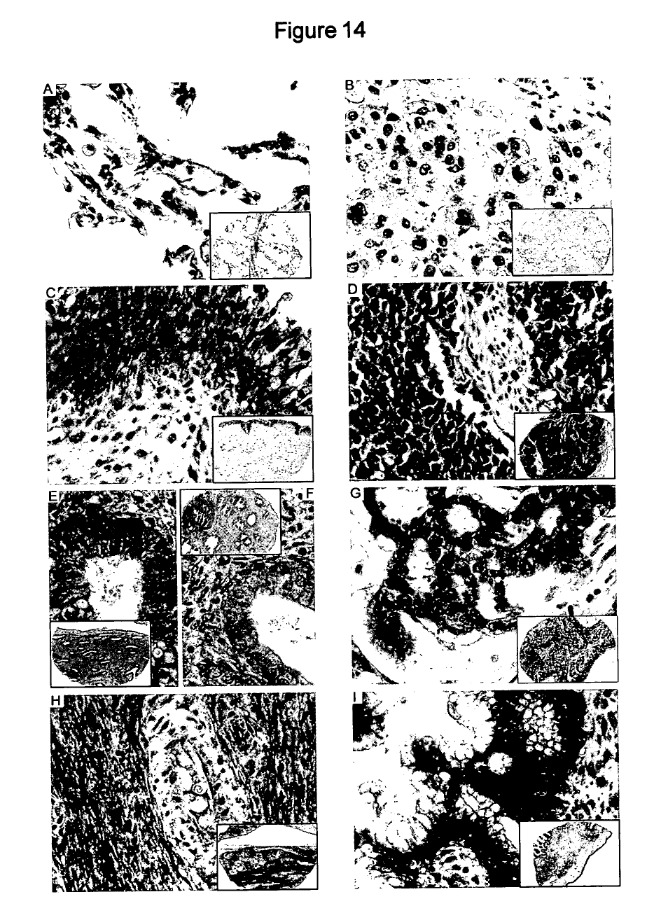

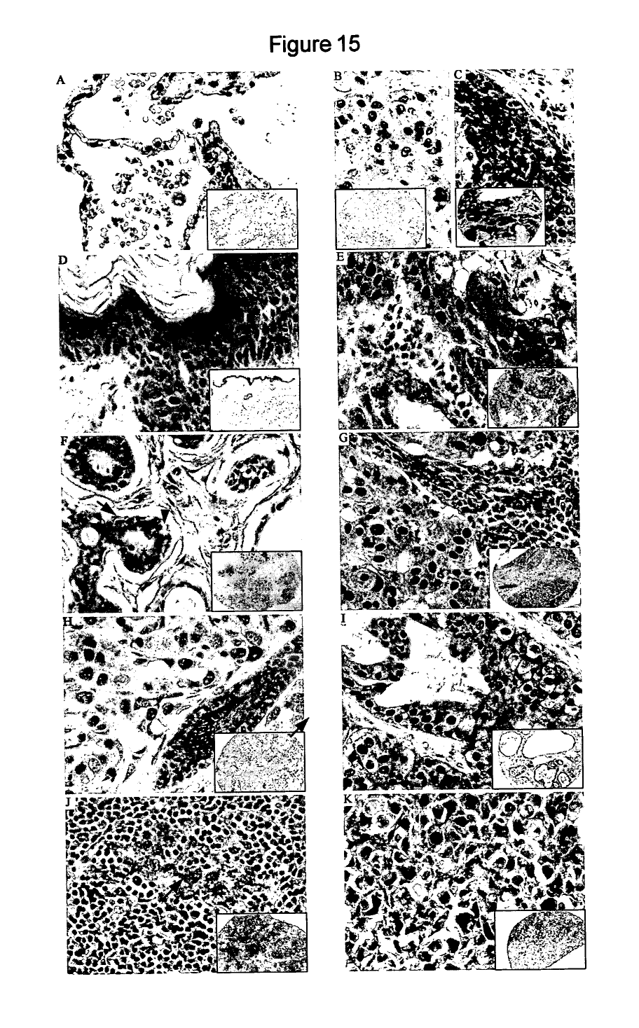

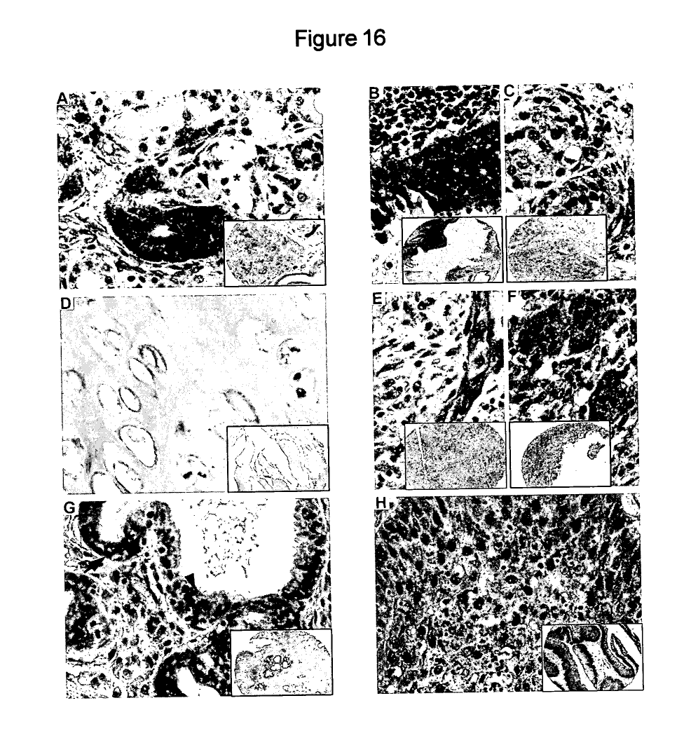

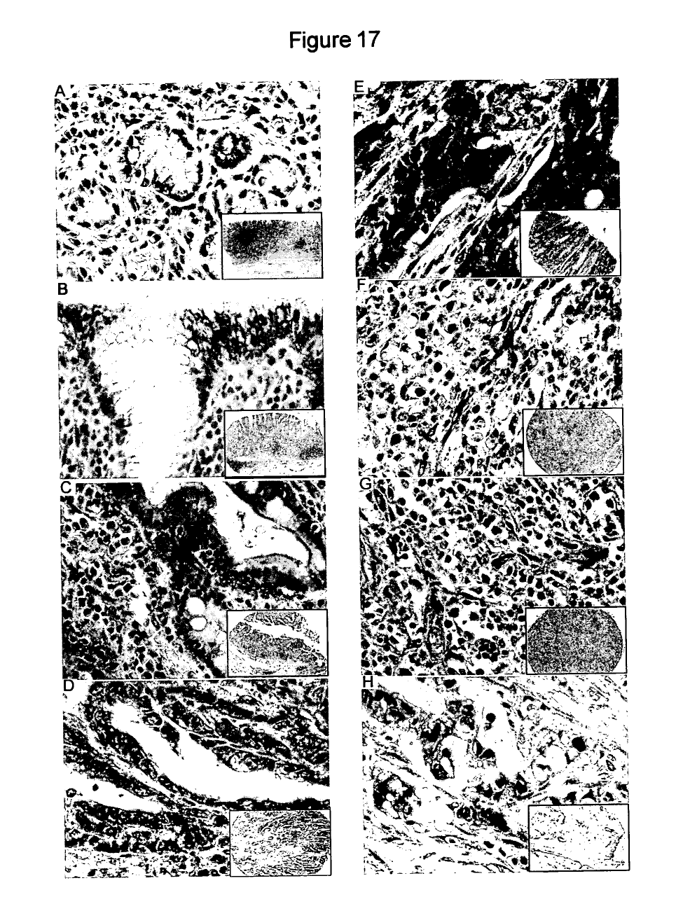

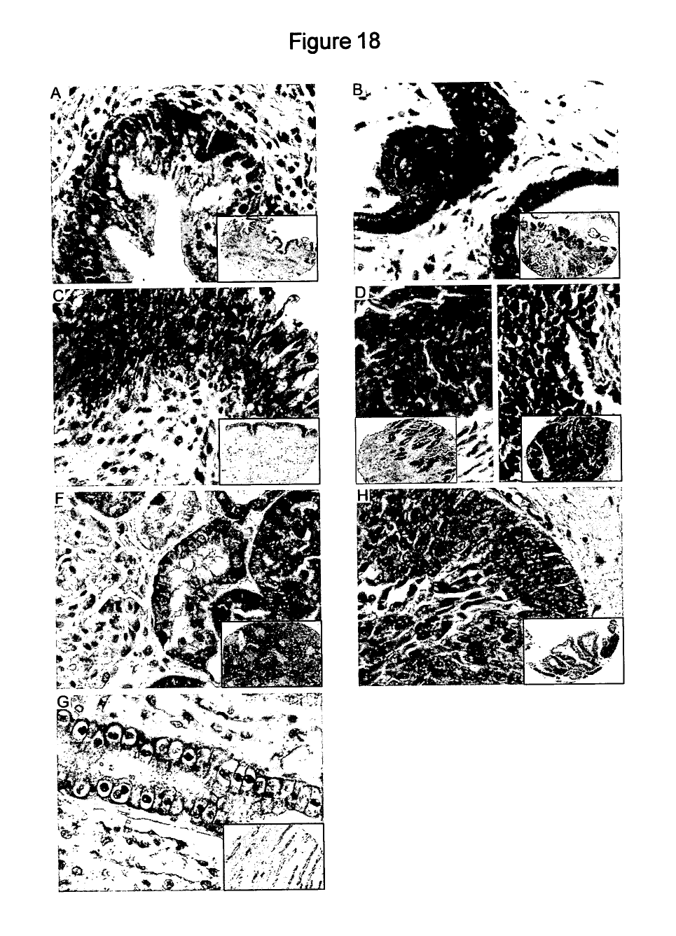

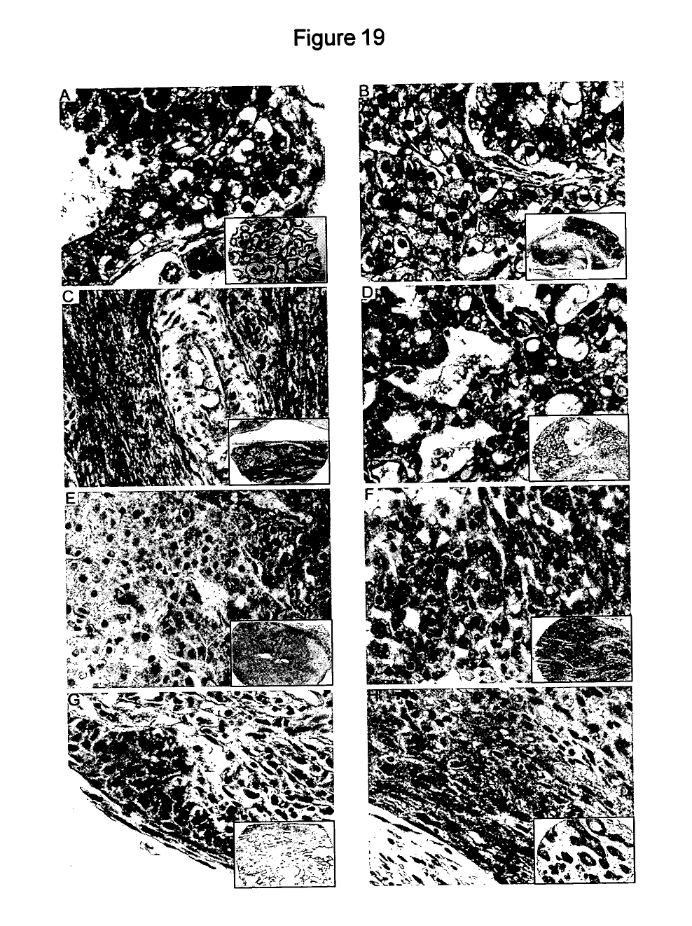

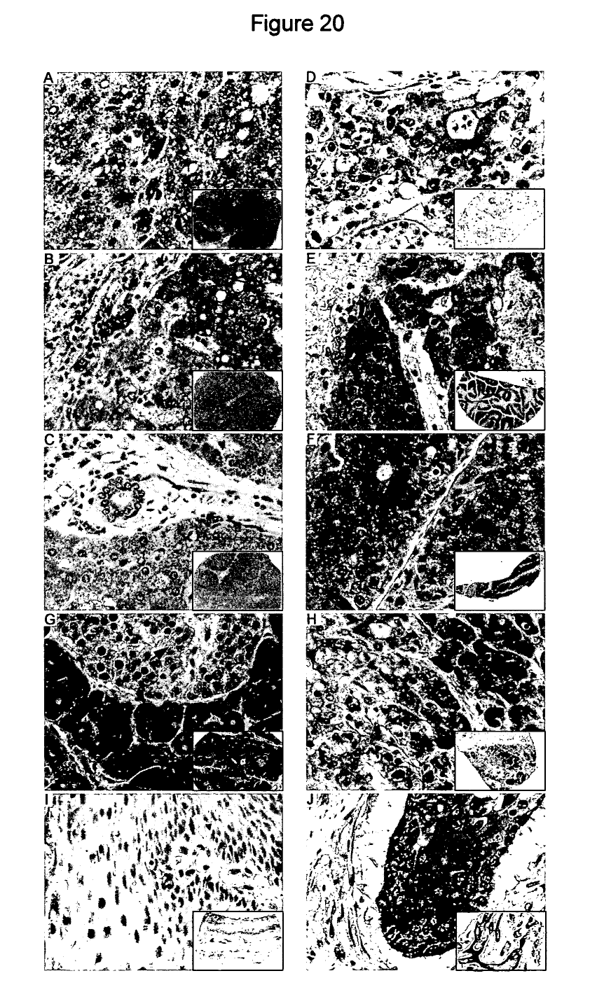

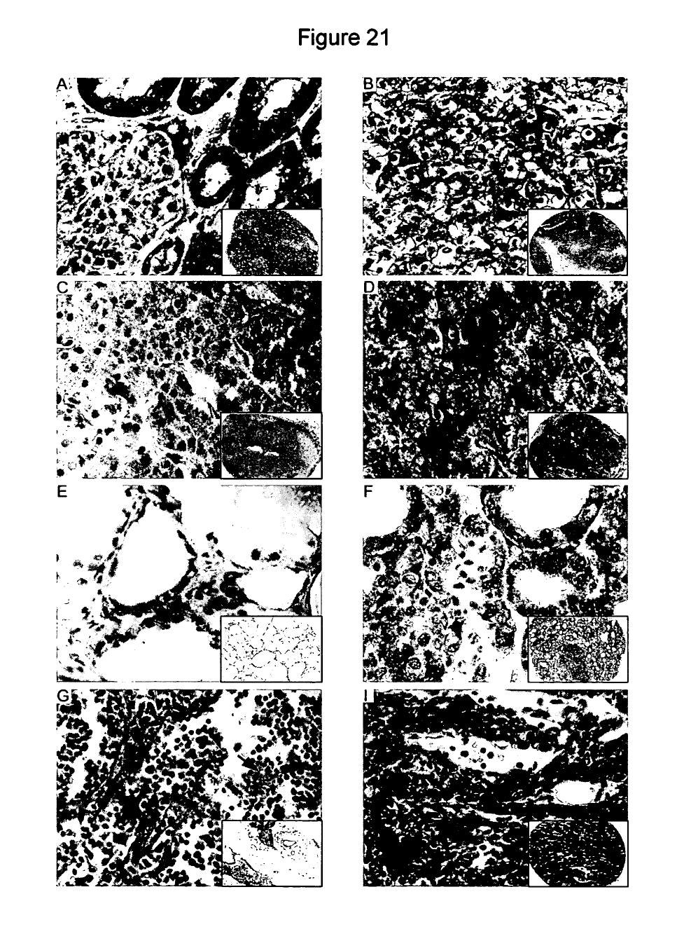

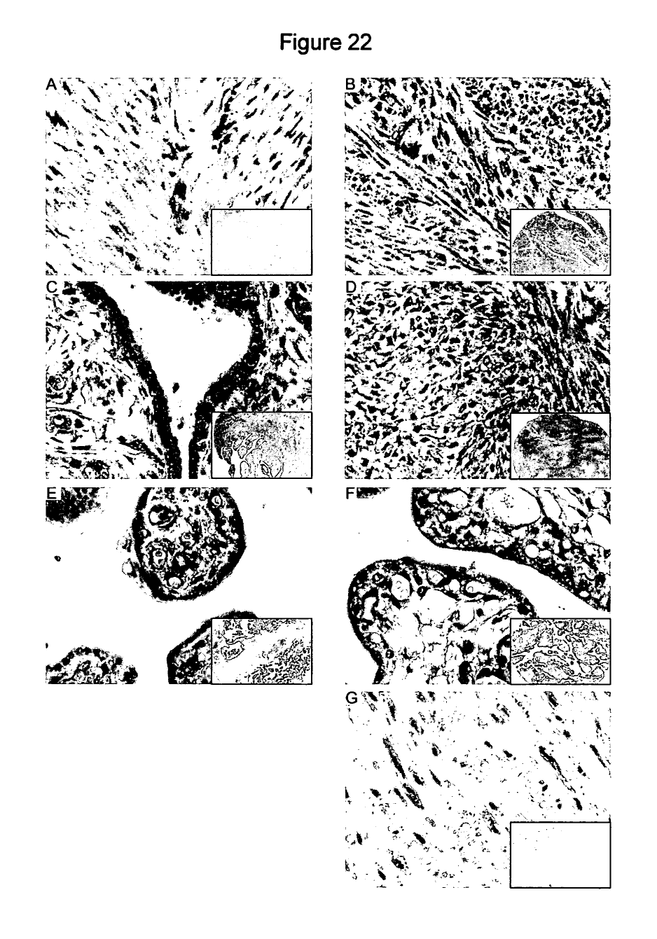

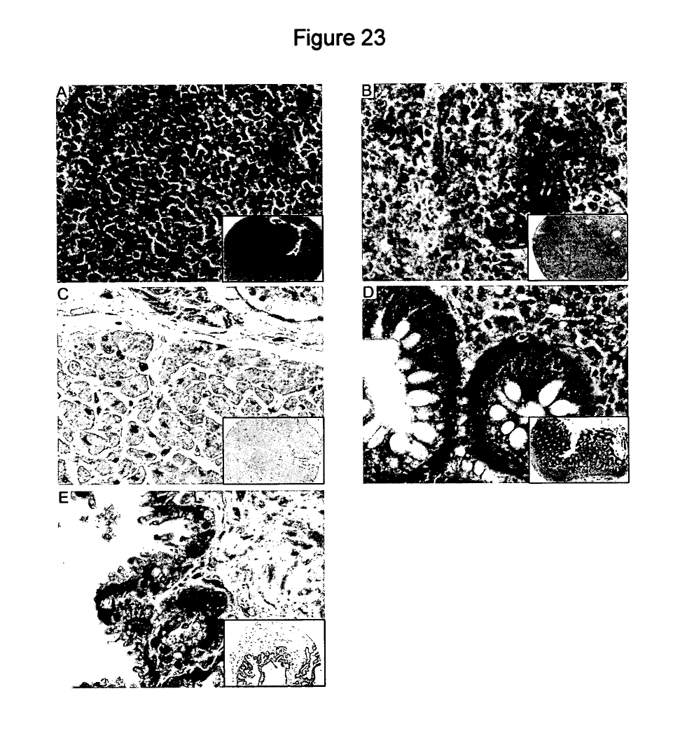

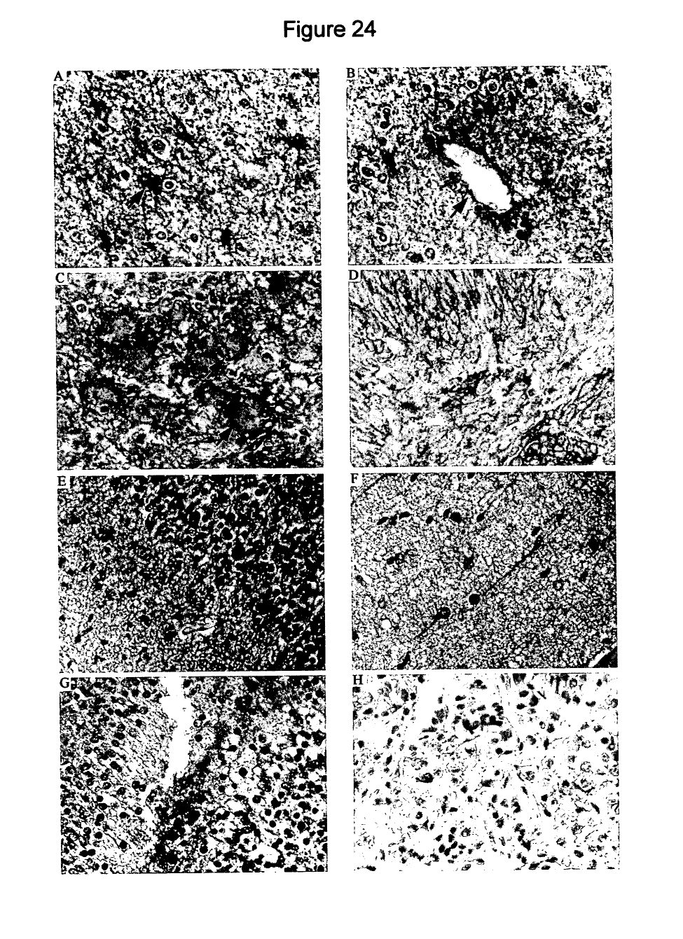

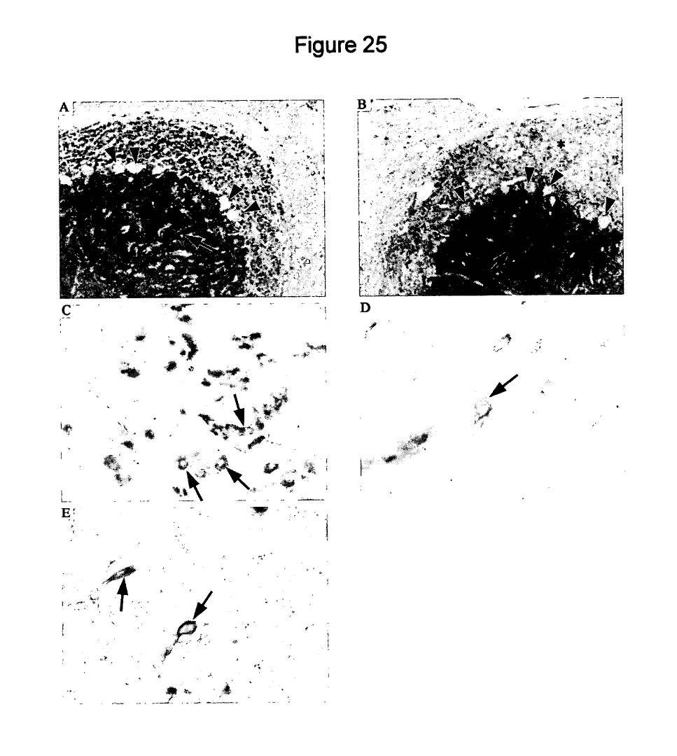

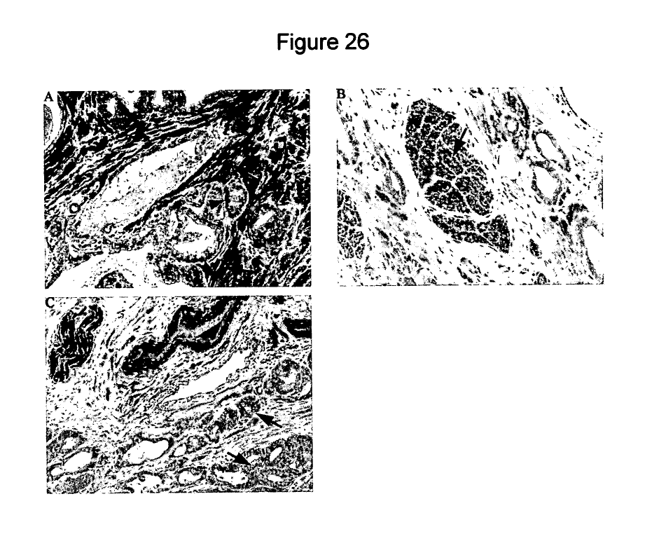

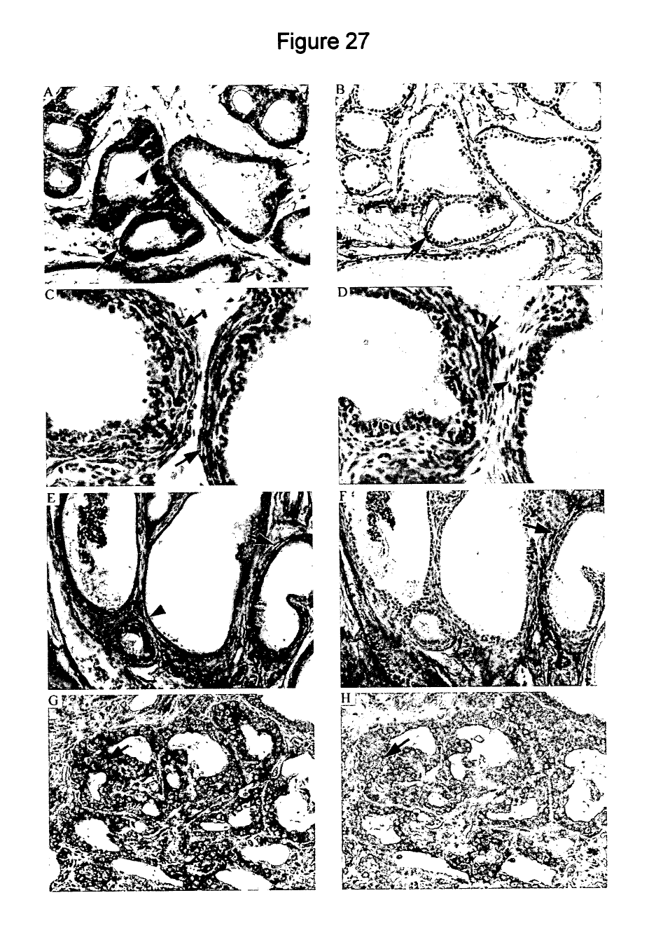

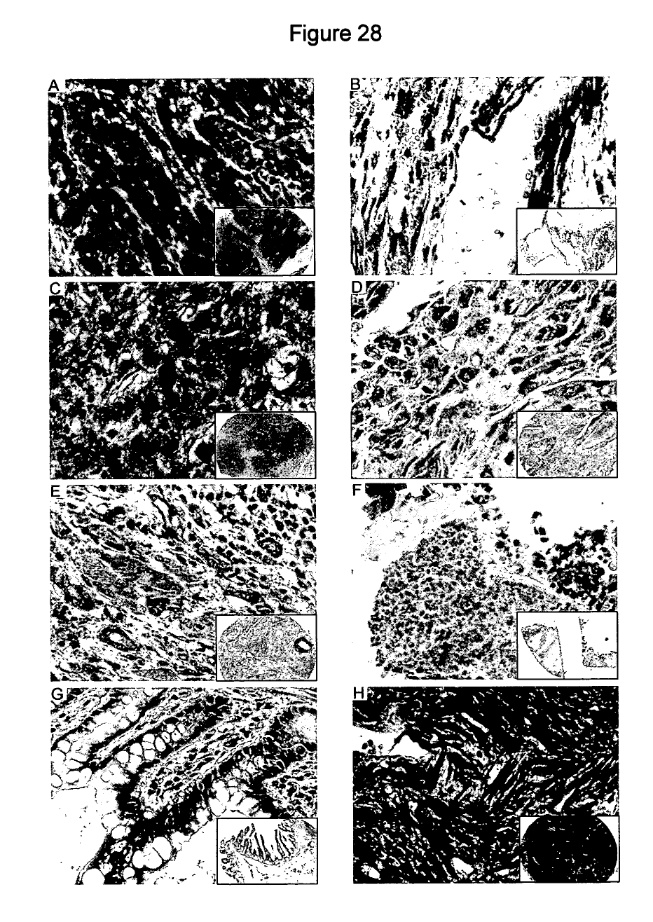

The activin β_{C} subunit protein was demonstrated to immunolocalise to most benign and malignant human organs studied. Both cytoplasmic and/or nuclear staining was commonly observed and changes in these patterns occurred between the benign and malignant state. FIGS. 12-28 fully describe the staining pattern and the descriptions below indicate some of the significant findings.

Endocrine organs (ovary, testes, adrenal gland and thyroid gland), as shown in FIG. 12, displayed strong activin β_{C} subunit protein localisation in malignancy. Staining in the adrenal and thyroid glands shows increased intensity in a malignant state.

Most of the adenocarcinomas of the stomach, colon and rectum (FIG. 13) and lung, endometrium, and mucinous ovary (FIG. 14) showed a pattern of both cytoplasmic and nuclear activin β_{C} subunit localisation. This staining pattern differed to the variable and predominantly cytoplasmic staining observed in the normal stomach, colon, rectum and lung. The localisation pattern in the ovary and endometrium displayed cytoplasmic activin β_{C} subunit localisation in the benign organ and both cytoplasmic and nuclear staining in malignancy. In addition, the intensity of activin β_{C} subunit protein in the proliferative phase of the benign endometrium was similar to the strong staining observed in malignancy.

Strong activin β_{C} subunit protein nuclear staining became apparent in the development of malignancy in the lung, skin, breast and lymph node (FIG. 15). Some nuclear staining was observed in some cells of the benign skin and breast, however stronger staining was displayed in malignant tissue. Similarly, cytoplasmic localisation of activin β_{C} subunit protein was observed in the normal salivary gland and nasal cavity however this staining showed strong nuclear localisation, as well as cytoplasmic, in malignancy (FIG. 16). In addition, little staining was observed in chondrosarcoma (a benign condition of the bone), however strong nuclear and cytoplasmic activin β_{C} subunit protein localisation was observed in malignancy. The normal stomach (FIGS. 13 and 17) displayed variable cytoplasmic localisation. In addition to the stomach adenocarcinomas described above, other stomach malignancies displayed nuclear localisation (and stromal localisation) including stomach signet ring cell carcinoma, stomach lymphoma and metastatic stomach carcinoma. The normal bladder and kidney have little nuclear staining however following the development of cancer, strong nuclear staining was observed (FIG. 18).

Some organs, such as the gallbladder, testis, adrenal, uterine cervix, pancreas and kidney had varying degrees of nuclear and cytoplasmic staining in the benign and malignant state (FIG. 18, 19, 20).

The esophagus, thyroid and thymus showed little or no staining in the normal tissue, however following the development of cancer increased activin β_{C} subunit protein localisation was observed (FIG. 20, 21).

Other tissues that immunolocalised activin β_{C} subunit protein included the myometrium, fallopian tube, placenta, tonsil, spleen, heart, appendix and seminal vesicle as well as benign disorders of the uterus and ovary (FIGS. 22 and 23).

The liver displayed strong activin β_{C} subunit protein localisation in the cytoplasm of heptatocytes. Interestingly in some liver cancers both cytoplasmic and nuclear localisation was observed (FIG. 20). However, tissue from one patient with cirrhosis did not localise the activin β_{C} subunit (results not shown).

Normal, damaged and malignant skin immunolocalised different patterns of activin β_{C} subunit protein staining. Both nuclear and cytoplasmic staining was observed in the normal skin and tumours including squamous cell and melanoma (FIG. 28).

The normal breast immunolocalised the activin β_{C} subunit and different breast tumours (residual infiltrating duct carcinoma, breast infiltrating lobular carcinoma, breast papillary carcinoma) also displayed cytoplasmic or nuclear localisation (FIG. 15).

The brain displays strong activin β_{C} subunit protein localisation in both the benign and malignant disorders. In particular, astrocytes, blood brain barrier and neurons strongly localise activin β_{C} subunit (FIG. 24). The endocrine cells of the sheep and human pituitary and the neuronal cells of the cerebellum, pre-optic area and hypothalamus display activin β_{C} subunit localization (FIG. 25). Strong localisation is also observed in tumour cells of the brain, in particular tumour cells in (I) glioblastoma of two patients and (II) meningioma of four patients.

In the benign prostate, activin β_{C} subunit protein localised to the basal cells, nerves and smooth muscle (FIG. 9). This pattern of staining was also observed in the benign region from a patient with prostate cancer, whereby the smooth muscle cells, nerves and basal cells were strongly positive (FIG. 26).

Evidence that the activin β_{C} subunit and TGF-β may heterodimerise is provided in FIG. 27. Both activin β_{C} subunit and TGF-β protein co-localise to the smooth muscle cells in serial tissue sections of the rat ventral prostate. In addition, in a patient with prostate cancer, both activin β_{C} subunit and TGF-β protein are localised to the same area of tumour cells in serial sections of tissue. Therefore activin β_{C}-TGF-β heterodimers have the capacity to be synthesised in the rodent and human prostate or any other organ in which these both growth factors are synthesised.

The discussion of prior art documents, acts, devices and the like is included in this specification solely for the purpose of providing a context for the present invention. It is not suggested or represented that any or all of these matters formed part of the prior art base or were common general knowledge in the field relevant to the present invention as it existed in the United States before the filing date of this application.

### REFERENCES

1. Luisi S, Florio P, Reis F M, Petraglia F 2001 Expression and secretion of activin A: possible physiological and clinical implications. Eur J Endocrinol 145:225-36.
2. McDowell N, Gurdon JB 1999 Activin as a morphogen in Xenopus mesoderm induction. Semin Cell Dev Biol 10:311-7
3. de Kretser D M, Hedger M P, Phillips D J 1999 Activin A and follistatin: their role in the acute phase reaction and inflammation. J Endocrinol 161:195-8.
4. Hubner G, Alzheimer C, Werner S 1999 Activin: a novel player in tissue repair processes. Histol Histopathol 14:295-304.
5. Lau A L, Nishimori K, Matzuk M M 1996 Structural analysis of the mouse activin beta C gene. Biochim Biophys Acta 1307:145-8
6. Hotten G, Neidhardt H, Schneider C, Pohl J 1995 Cloning of a new member of the TGF-beta family: a putative new activin beta C chain. Biochem Biophys Res Commun 206:608-13
7. Loveland K L, McFarlane J R, de Kretser D M 1996 Expression of activin beta C subunit mRNA in reproductive tissues. J Mol Endocrinol 17:61-5.
8. Oda S, Nishimatsu S, Murakami K, Ueno N 1995 Molecular cloning and functional analysis of a new activin beta subunit: a dorsal mesoderm-inducing activity in Xenopus. Biochem Biophys Res Commun 210:581-8
9. Fang J, Yin W, Smiley E, Wang S Q, Bonadio J 1996 Molecular cloning of the mouse activin beta E subunit gene. Biochem Biophys Res Commun 228:669-74
10. O'Bryan M K, Sebire K L, Gerdprasert O, Hedger M P, Hearn M T, de Kretser D M 2000 Cloning and regulation of the rat activin betaE subunit. J Mol Endocrinol 24:409-18.
11. Esquela A F, Zimmers T A, Koniaris L G, Sitzmann J V, Lee S J 1997 Transient down-regulation of inhibin-betaC expression following partial hepatectomy. Biochem Biophys Res Commun 235:553-6
12. Zhang Y Q, Shibata H, Schrewe H, Kojima 11997 Reciprocal expression of mRNA for inhibin betaC and betaA subunits in hepatocytes. Endocr J 44:759-64
13. Lebrun J J, Takabe K, Chen Y, Vale W 1999 Roles of pathway-specific and inhibitory Smads in activin receptor signaling. Mol Endocrinol 13:15-23.
14. Wrana J L, Attisano L 2000 The Smad pathway. Cytokine Growth Factor Rev 11:5-13.
15. Pangas S A, Woodruff T K 2000 Activin Signal Transduction Pathways. Trends Endocrinol Metab 11:309-314.
16. Chen X, Rubock M J, Whitman M 1996 A transcriptional partner for MAD proteins in TGF-beta signalling. Nature 383:691-6.
17. Chen X, Weisberg E, Fridmacher V, Watanabe M, Naco G, Whitman M 1997 Smad4 and FAST-1 in the assembly of activin-responsive factor. Nature 389:85-9.
18. Schmitt J, Hotten G, Jenkins N A, Gilbert D J, Copeland N G, Pohl J, Schrewe H 1996 Structure, chromosomal localization, and expression analysis of the mouse inhibin/activin beta C (Inhbc) gene. Genomics 32:358-66.
19. Kron R, Schneider C, Hotten G, Bechtold R, Pohl J 1998 Expression of human activin C protein in insect larvae infected with a recombinant baculovirus. J Virol Methods 72:9-14.
20. Kohler G, Milstein C 1975 Continuous cultures of fused cells secreting antibody of predefined specificity. Nature 256:495-7.
21. Kozbar 1983 Immunology Today 4:72
22. Cole 1985 Monoclonal Antibodies and Cancer Therapy. In. Alan R. Liss, Inc:77-96
23. Morgan D O, Roth R A 1988 Analysis of intracellular protein function by antibody injection. Immunol Today 9:84-8.
24. Burke B, Warren G 1984 Microinjection of mRNA coding for an anti-Golgi antibody inhibits intracellular transport of a viral membrane protein. Cell 36:847-56.
25. McFarlane J R, Foulds L M, Pisciotta A, Robertson D M, de Kretser DM 1996 Measurement of activin in biological fluids by radioimmunoassay, utilizing dissociating agents to remove the interference of follistatin. Eur J Endocrinol 134:481-9.
26. Hershkowitz 1987 Nature 329:219-222.
27. Stein C A, Cohen J S 1988 Oligodeoxynucleotides as inhibitors of gene expression: a review. Cancer Res 48:2659-68.
28. van der Krol A R M J, Stuitje A R. 1988 Modulation of eukaryotic gene expression by complementary RNA or DNA sequences. Biotechniques 6: 958-976
29. Landis S H, Murray T, Bolden S, Wingo P A 1998 Cancer statistics, 1998. CA Cancer J Clin 48:6-29.
30. Bostwick D G, Pacelli A, Lopez-Beltran A 1996 Molecular biology of prostatic intraepithelial neoplasia. Prostate 29:117-34.
31. Lee C, Sintich S M, Mathews E P, Shah A H, Kundu S D, Perry K T, Cho J S, Ilio K Y, Cronauer M V, Janulis L, Sensibar J A 1999 Transforming growth factor-beta in benign and malignant prostate. Prostate 39:285-90.
32. de Caestecker M P, Piek E, Roberts A B 2000 Role of transforming growth factor-beta signaling in cancer. J Natl Cancer Inst 92:1388-402.
33. Markowitz S D, Roberts A B 1996 Tumor suppressor activity of the TGF-beta pathway in human cancers. Cytokine Growth Factor Rev 7:93-102.
34. Thomas T Z, Wang H, Niclasen P, O'Bryan M K, Evans L W, Groome N P, Pedersen J, Risbridger G P 1997 Expression and localization of activin subunits and follistatins in tissues from men with high grade prostate cancer. J Clin Endocrinol Metab 82:3851-8.
35. Evans L W, Muttukrishna S, Groome N P 1998 Development, validation and application of an ultra-sensitive two-site enzyme immunoassay for human follistatin. J Endocrinol 156:275-82
36. McPherson S J, Mellor S L, Wang H, Evans L W, Groome N P, Risbridger G P 1999 Expression of activin A and follistatin core proteins by human prostate tumor cell lines. Endocrinology 140:5303-5309
37. Knight P G, Muttukrishna S, Groome N P 1996 Development and application of a two-site enzyme immunoassay for the determination of ‘total’ activin-A concentrations in serum and follicular fluid. J Endocrinol 148:267-79
38. Gurney M E, Pu H, Chiu A Y, Dal Canto M C, Polchow C Y, Alexander D D, Caliendo J, Hentati A, Kwon Y W, Deng H X, et al. 1994 Motor neuron degeneration in mice that express a human Cu,Zn superoxide dismutase mutation. Science 264(5166):1772-5.

Finally, the invention as hereinbefore described is susceptible to variations, modifications and/or additions other than those specifically described and it is to be understood that the invention includes all such variations, modifications and/or additions which fall within the scope of the description as hereinbefore described.

1. A method of modulating the formation of an activin dimer in a cell or biological sample, the method including controlling levels or bioactivity of activin βC in the cell or biological sample.

2. A method according to claim 1 wherein the formation of activin dimers is formed by the dimerisation of activin subunits selected from the group consisting of βA, βB, βC, βD or βE, or combinations thereof.

3. A method according to claim 1 wherein the activin dimer is a homodimer selected from the group consisting of activin A (βA-βA), activin B (βB-βB), activin C (βC-βC), activin D (βD-βD) or activin E (βE-βE).

4. A method according to claim 1 wherein the activin dimer is a heterodimer selected from the group consisting of activin AB (βA-βB), activin AC (βA-βC), activin AD (βA-βD), activin AE (βA-βE), activin BC (βB-βC), activin BD (βB-βD), activin BE (βB-βE), activin CD (βC-βD), activin CE (βC-βE) or activin ED (βE-βD).

5. A method according to claim 1 wherein the activin dimer is activin A (βA-βA), AB (βA-βB) or B (βB-βB).

6. A method according to claim 1 wherein the activin dimer is activin A (βA-βA).

7. A method according to claim 1 wherein the levels or bioactivity of activin βC are controlled by increasing or decreasing endogeneous or exogeneous activin βC.

8. A method according to claim 1 wherein the levels or bioactivity of activin βC are increased or decreased by altering expression and/or activity of βC.

9. A method according to claim 1 wherein the modulating the formation of the activin dimer includes inhibiting the formation of an activin dimer in a cell or biological sample, the method including increasing levels or bioactivity of activin βC in the cell or biological sample.

10. A method according to claim 9 wherein the level or bioactivity of activin βC is increased by introducing exogeneous βC or increasing expression and/or activity of endogeneous or exogeneous βC in the cell or biological sample.

11. A method according to claim 9 wherein the formation of activin dimers is formed by the dimerisation of activin subunits selected from the group consisting of βA, βB, βC, βD or βE, or combinations thereof.

12. A method according to claim 9 wherein the activin dimer is a homodimer selected from the group consisting of activin A (βA-βA), activin B (βB-βB), activin C (βC-βC), activin D (βD-βD) or activin E (βD-βE).

13. A method according to claim 9 wherein the activin dimer is a heterodimer selected from the group consisting of activin AB (βA-βB), activin AC (βA-βC), activin AD (βA-βD), activin AE (βA-βE), activin BC (βB-βC), activin BD (βB-βD), activin BE (βB-βE), activin CD (βC-βD), activin CE (βC-βE) or activin ED (βE-βD).

14. A method according to claim 9 wherein the activin dimer is activin A (βA-βA), AB (βA-βB) or B (βB-βB).

15. A method according to claim 9 wherein the activin dimer is activin A (βA-βA).

16. A method according to claim 1 wherein the modulating the formation of the activin dimer includes inducing the formation of an activin dimer in a cell or biological sample, the method including decreasing levels or bioactivity of activin βC in the cell or biological sample.

17. A method according to claim 16 wherein the level or bioactivity of activin βC is decreased by decreasing expression and/or activity of endogeneous or exogeneous βC in the cell or biological sample.

18. A method according to claim 16 wherein the level or bioactivity of activin βC is decreased by an activin βC inhibitory molecule selected from the group including an antagonist of activin βC, an antibody against activin βC, an activin βC antisense oligonucleotide or an agent that decreases the expression of activin βC.

19. A method according to claim 16 wherein the level or bioactivity of activin βC is decreased by an antibody or fragment of the antibody that is reactive to an epitope of activin βC or precursor protein thereof.

20. A method according to claim 16 wherein the level or bioactivity of activin βC is decreased by an antibody which is reactive to an epitope of activin βC having an amino acid sequence of VPTARRPLSLLYYDRDSNIVKTDIPDMVVEAC [SEQ ID NO.: 1] or equivalent thereof.

21. A method according to claim 16 wherein the bioactivity of activin βC is decreased by follistatin.

22. A method according to claim 16 wherein the formation of activin dimers is formed by the dimerisation of activin subunits selected from the group consisting of βA, βB, βC, βD or βE, or a combination thereof.

23. A method according to claim 16 wherein the activin dimer is a homodimer selected from the group consisting of activin A (βA-βA), activin B (βB-βB), activin C (βC-βC), activin D (βD-βD) or activin E (βD-βE).

24. A method according to claim 16 wherein the activin dimer is a heterodimer selected from the group consisting of activin AB (βA-βB), activin AC (βA-βC), activin AD (βA-βD), activin AE (βA-βE), activin BC (βB-βC), activin BD (βB-βD), activin BE (βB-βE), activin CD (βC-βD), activin CE (βC-βE) or activin ED (βE-βD).

25. A method according to claim 16 wherein the activin dimer is activin A (βA-βA), AB (βA-βB) or B (βB-βB).

26. A method according to claim 16 wherein the activin dimer is activin A (βA-βA).

27. A method according to claim 1 wherein the cell is selected from the group including normal, cancer or tumor cells of the prostate, fibroblast, epidermal, dermal, placental, ovary, testis, adrenal, brain and neural tissue, kidney, pancreas, heart, neural cells, muscle cells, pituitary, thyroid gland, stomach, colon, lung, urinary bladder, endometrium, breast, lymph node, skin, salivary gland, bone, nasal cavity, duodenum, gallbladder, uterine cervix, thymus, placenta, fallopian tube, uterus, tonsil, spleen, appendix, seminal vesicle, larynx, tongue, small instestine, rectum, esophagus, myometriumand soft tissue.

28. A method according to claim 1 wherein the cell is selected from the group including normal, cancer or tumor cells of the liver.

29. A method according to claim 1 wherein the cell is a prostate cancer cell.

30. A method according to claim 1 wherein the biological sample is selected from the group including serum, tissue extracts, body fluids, cell culture medium, extracellular medium, supernatants, biopsy specimens or resected tissue.

31. An antibody which recognises an epitope of activin βC having the amino acid sequence of VPTARRPLSLLYYDRDSNIVKTDIPDMVVEAC [SEQ ID NO.: 1] or equivalent thereof.

32. A method of detecting an activin βC subunit and/or an activin dimer including an activin βC subunit, said method including detecting the activin βC subunit and/or and activin dimer including an activin βC subunit with an antibody that recognises an epitope of an activin βC subunit.

33. A method according to claim 32 wherein the antibody recognises an epitope of activin βC having the amino acid sequence of VPTARRPLSLLYYDRDSNIVKTDIPDMVVEAC [SEQ ID NO.: 1] or equivalent thereof.

34. A method according to claim 32 wherein the activin dimer is selected from the group consisting of activin AC (βA-βC), activin BC (βB-βC), activin C (βC-βC), activin CD (βC-βD) or activin CE (βC-βE).

35. A method according to claim 32 wherein the activin dimer is activin AC (βA-βC).

36. A method according to claim 32 wherein the activin dimer is CC (βC-βC).

37. A method according to claim 32 wherein the activin subunit is βC monomer.

38. A method of detecting an activin βC dimer in a biological sample, the method including the steps of:
(a) contacting a first antibody that recognises an epitope of a first activin β subunit with a biological sample;
(b) allowing the first antibody to bind to a first activin β subunit in the sample;
(c) washing the sample to substantially remove any unbound material in the sample;
(d) contacting the sample with a second antibody that recognises an epitope of a second activin β subunit, wherein the second antibody is tagged with a labelling agent; and
(e) detecting the labelling agent to identify an activin βC dimer in the biological sample, wherein the first or second antibody recognises an epitope of an activin βC subunit according to claim 31.

39. A method according to claim 38 which detects an activin dimer selected from the group consisting of activin AC (βA-βC), activin BC (βB-βC), activin C (βC-βC), activin CD (βC-βD) or activin CE (βC-βE).

40. A method according to claim 38 wherein the biological sample is selected from the group including serum, tissue extracts, body fluids, cell culture medium, extracellular medium, supernatants, biopsy specimens or resected tissue.

41. A method according to claim 38 further including adding a dissociating agent to the sample to remove binding proteins.

42. method according to claim 38 wherein the dissociating agent is selected from the group including SDS, sodium deoxycholate and Tween 20.

43. A method for detecting a propensity for an activin dimer to form in a cell or biological sample the said method comprising detecting a level or bioactivity of activin βC in the cell or biological sample.

44. A method according to claim 43 wherein the activin dimer is selected from the group including activin AC (βA-βC), activin BC (βB-βC), activin C (βC-βC), activin CD (βC-βD) or activin CE (βC-βE).

45. A method according to claim 43 wherein the activin dimer is activin AC (βA-βC).

46. A method according to claim 43 wherein the cell is selected from the group including normal, cancer or tumor cells of the prostate, fibroblast, epidermal, placental, ovary, testis, adrenal, brain and neural tissue, kidney, pancreas, heart, neural cells, muscle cells, pituitary, thyroid gland, stomach, colon, lung, urinary bladder, endometrium, breast, lymph node, skin, salivary gland, bone, nasal cavity, duodenum, gallbladder, uterine cervix, thymus, placenta, fallopian tube, uterus, tonsil, spleen, appendix, seminal vesicle, larynx, tongue, small intestine, rectum, esophagus, myometrium and soft tissue.

47. A method according to claim 43 wherein the cell is selected from the group including normal, cancer or tumor cells of the liver.

48. A method of diagnosing and/or prognosing a disease or condition associated with activin dimer or dimer formation, the method including detecting an activin βC subunit and/or an activin dimer including an activin βC subunit in a cell or biological sample of a subject.

49. A method of diagnosing and/or prognosinga disease or condition associated with activin dimer formation, the method including detecting an activin βC subunit and/or an activin dimer including an activin βC subunit in a cell or biological sample of a subject according to the method of claim 32.

50. A method according to claim 48 wherein the antibody recognises an epitope of activin βC.

51. A method according to claim 48 wherein the antibody recognises an epitope of activin βC having the amino acid sequence of VPTARRPLSLLYYDRDSNIVKTDIPDMVVEAC [SEQ ID NO.: 1] or equivalent thereof.

52. A method according to claim 48 wherein the activin βC dimer formation detected is activin AC (βA-βC), activin BC (βB-βC), activin C (βC-βC), activin CD (βC-βD) or activin CE (βC-βE).

53. A method according to claim 48 wherein the activin dimer is activin AC (βA-βC).

54. A method according to claim 48 wherein the disease or condition associated with activin dimers or dimer formation is selected from the group including diseases or conditions of the prostate, testis, ovary, pancreas, kidney, heart, reproductive organs, skeletal muscle, pituitary, thyroid gland, brain and neural tissue, stomach, colon, lung, urinary bladder, endometrium, breast, lymph node, skin, salivary gland, bone, nasal cavity, duodenum, gallbladder, uterine cervix, thymus, placenta, fallopian tube, uterus, tonsil, spleen, appendix, seminal vesicle, larynx, tongue, small intestine, adrenal, rectum, esophagus, myometrium and soft tissue.

55. A method according to claim 48 wherein the disease or condition associated with activin dimers or dimer formation is a disease or condition of the liver.

56. A method according to claim 48 wherein the disease or condition is selected from the group including ovarian cancer, testicular disorder including testicular cancer, endometrial cancer, prostate cancer or prostate enlargement including benign prostatic hyperplasia, inflammatory conditions including rheumatoid arthritis, pneumonia, gastrointestinal infection.

57. A method according to claim 48 wherein the disease or condition is liver disease including cirrhosis, cancer or hepatitis.

58. A method according to claim 48 wherein the disease or condition is cancer.

59. A method according to claim 48 wherein the disease or condition is prostate cancer.

60. A method of diagnosing and/or prognosing a disease or condition associated with activin dimer formation the method including detecting an activin βC subunit and/or an activin dimer including an activin βC subunit in a cell or biological sample of a subject according to the method of claim 38.

61. A method of diagnosing and/or prognosinga disease or condition associated with activin dimer formation the method including detecting a propensity for activin dimer formation according to claim 43.

62. A composition for detecting an activin βC subunit and/or an activin dimer including an activin βC subunit in a cell or biological sample, wherein the composition includes an antibody that recognises an epitope of an activin βC subunit, and a suitable diluent, excipient or carrier.

63. A composition according to claim 62 wherein the antibody recognises an epitope of activin βC that includes the amino acid sequence VPTARRPLSLLYYDRDSNIVKTDIPDMVVEAC [SEQ ID NO.: 1] or equivalent thereof.

64. A composition for diagnosing and/or prognosing a disease or condition associated with activin dimer formation, wherein the composition includes an antibody that recognises an epitope of an activin βC subunit, and a suitable diluent, excipient or carrier.

65. A composition according to claim 64 wherein the antibody recognises an epitope of activin βC that includes the amino acid sequence VPTARRPLSLLYYDRDSNIVKTDIPDMVVEAC [SEQ ID NO.: 1] or equivalent thereof.

66. A kit for detecting an activin βC dimer in a cell or biological sample, wherein the kit includes a first antibody that recognises an epitope of a first activin β subunit, a second antibody that recognises an epitope of a second activin β subunit, and a labelling agent for tagging the second antibody, wherein the first or second antibody recognises an epitope of an activin βC subunit.

67. A kit according to claim 66 wherein the antibody recognises an epitope of activin βC that includes the amino acid sequence VPTARRPLSLLYYDRDSNIVKTDIPDMVVEAC [SEQ ID NO.: 1] or equivalent thereof.

68. A kit for diagnosing and/or prognosing a disease or condition associated with activin dimer formation, wherein the kit includes a first antibody that recognises an epitope of a first activin β subunit, a second antibody that recognises an epitope of a second activin β subunit, and a labelling agent for tagging the second antibody, wherein the first or second antibody recognises an epitope of an activin βC subunit.

69. A kit according to claim 68 wherein the antibody recognises an epitope of activin βC that includes the amino acid sequence VPTARRPLSLLYYDRDSNIVKTDIPDMVVEAC [SEQ ID NO.: 1] or equivalent thereof.

70. A method of treating or preventing a disease or condition associated with activin dimer formation, the method including modulating the formation of an activin dimer in a cell or biological sample, the method including controlling levels or bioactivity of activin βC in the cell or biological sample.

71. A method according to claim 70 wherein the levels or bioactivity of activin βC are controlled by increasing or decreasing endogeneous or exogeneous activin βC.

72. A method according to claim 70 wherein the levels or bioactivity of activin βC are increased or decreased by altering expression and/or activity of βC.

73. A method according to claim 70 wherein the modulating the formation of the activin dimer includes inducing the formation of an activin dimer in a cell or biological sample, the method including decreasing levels or bioactivity of activin βC in the cell or biological sample.

74. A method according to claim 73 wherein the level or bioactivity of activin βC is decreased by decreasing expression and/or activity of endogeneous or exogeneous βC in the cell or biological sample.

75. A method according to claim 73 wherein the level or bioactivity of activin βC is decreased by an activin βC inhibitory molecule selected from the group including an antagonist of activin βC, an antibody against activin βC, an activin βC antisense oligonucleotide or an agent that decreases the expression of activin βC.

76. A method according to claim 73 wherein the level or bioactivity of activin βC is decreased by an antibody or fragment of the antibody that is reactive to an epitope of activin βC or precursor protein thereof.

77. A method according to claim 73 wherein the level or bioactivity of activin βC is decreased by an antibody which is reactive to an epitope of activin βC having an amino acid sequence of VPTARRPLSLLYYDRDSNIVKTDIPDMVVEAC [SEQ ID NO.: 1] or equivalent thereof.

78. A method according to claim 73 wherein the disease or condition associated with activin dimers or dimer formation is selected from the group including diseases or conditions of the prostate, testis, ovary, pancreas, kidney, heart, reproductive organs, skeletal muscle, pituitary, thyroid gland, stomach, colon, lung, urinary bladder, brain and neural tissue, endometrium, breast, lymph node, skin, salivary gland, bone, nasal cavity, duodenum, gallbladder, uterine cervix, thymus, placenta, fallopian tube, uterus, tonsil, spleen, appendix, seminal vesicle, larynx, tongue, small intestine, adrenal, rectum, esophagusand soft tissue.

79. A method according to claim 73 wherein the disease or condition associated with activin dimers or dimer formation is a disease or condition of the liver.

80. A method according to claim 73 wherein the disease or condition is selected from the group including ovarian cancer, testicular disorder including testicular cancer, endometrial cancer, prostate cancer or prostate enlargement including benign prostatic hyperplasia, inflammatory conditions including rheumatoid arthritis, pneumonia, gastrointestinal infection.

81. A method according to claim 73 wherein the disease or condition is liver disease including cirrhosis, cancer or hepatitis.

82. A method according to claim 73 wherein the disease or condition is cancer.

83. A method according to claim 73 wherein the disease or condition is prostate cancer.

84. A method according to claim 70 wherein the modulating the formation of the activin dimer includes inhibiting the formation of an activin dimer in a cell or biological sample, the method including increasing levels or bioactivity of activin βC in the cell or biological sample.

85. A method according to claim 70 wherein the level or bioactivity of activin βC is increased by increasing expression and/or activity of endogeneous or exogeneous βC in the cell or biological sample.

86. A method according to claim 84 wherein the condition is a regeneration of tissue or a halting of degradation of tissue.

87. A pharmaceutical composition for treating, preventing or diagnosing and/or prognosing a disease or condition associated with activin dimer formation, the composition including an effective amount of activin or an activin βC inhibitory molecule, and a suitable pharmaceutically acceptable diluent, excipient or carrier.

88. A pharmaceutical composition according to claim 87 wherein the inhibitory molecule is an antibody which is reactive to an epitope of activin βC having an amino acid sequence of VPTARRPLSLLYYDRDSNIVKTDIPDMVVEAC [SEQ ID NO.: 1] or equivalent thereof.


SEQUENCE LISTING (9 sequences)

SEQ ID NO: 1 - protein, 32 aa, artificial
  synthetic sequence
VPTARRPLSLLYYDRDSNIVKTDIPDMVVEAC

SEQ ID NO: 2 - DNA, 12 bp, artificial
  linker
aattccagccag

SEQ ID NO: 3 - DNA, 12 bp, artificial
  linker
catggtggctgg

SEQ ID NO: 4 - DNA, 32 bp, artificial
  primer
ccagccatggcctcctcattgcttctggcctt

SEQ ID NO: 5 - DNA, 25 bp, artificial
  primer
gtagtcgaaacgactctgtccggag

SEQ ID NO: 6 - DNA, 24 bp, artificial
  primer
gccctgtgtccagagctgctttga

SEQ ID NO: 7 - DNA, 25 bp, artificial
  primer
cgtttgtggtctaagtggctgctcc

SEQ ID NO: 8 - DNA, 25 bp, artificial
  primer
ctggagctggtacttgaaggccagg

SEQ ID NO: 9 - DNA, 31 bp, artificial
  primer
ggacacccacgtcaatcagattcgaaccata